(19) United States
(12) Patent Application Publication
(10) Pub. No.: US 2008/0160027 A1
(43) Pub. Date: Jul. 3, 2008

(54) CHLAMYDIA PNEUMONIAE VACCINE AND METHODS FOR ADMINISTERING SUCH A VACCINE

(76) Inventors: Kathryn Frances Sykes, Tempe, AZ (US); Alexandre Yurievich Borovkov, Tempe, AZ (US); Bernhard Kaltenboeck, Auburn, AL (US); Yihang Li, Auburn, AL (US); Chengming Wang, Auburn, AL (US)

Correspondence Address: Andrus, Sceales, Starke & Sawall, LLP;Suite 1100, 100 East Wisconsin Avenue, Milwaukee, WI, 53202, US

(21) Appl. No.: 11/950,173

(22) Filed: Dec. 4, 2007

Related U.S. Application Data
(60) Provisional application No. 60/875,920, filed on Dec. 19, 2006.
(60) Provisional application No. 60/872,694, filed on Dec. 4, 2006.

(51) Int. Cl. A61K 39/02; C07K 16/00; G01N 33/566; A61P 31/00

(52) U.S. Cl. 424/139.1; 530/387.9; 436/501; 435/7.92

(57) ABSTRACT

The present application relates to antigens and nucleic acids encoding such antigens obtainable by screening the Chlamydia pneumoniae genome. In more specific aspects, the present application relates to methods of isolating such antigens and nucleic acids and the methods of using such isolated antigens for producing immune responses. The ability of an antigen to produce an immune response may be employed by vaccination or antibody preparation techniques.

### CROSS REFERENCE TO RELATED APPLICATIONS

This application claims priority to co-pending U.S. Provisional Patent Application Ser. Nos. 60/875,920, filed on Dec. 19, 2006 and 60/872,694, filed on Dec. 4, 2006. The entire text of the above referenced disclosures are specifically incorporated herein by reference.

### STATEMENT REGARDING FEDERALLY SPONSORED RESEARCH OR DEVELOPMENT

The Government may own rights in the present invention pursuant to National Institutes of Health (NIH) Grant No. AI47202.

### BACKGROUND AND SUMMARY

The present application relates generally to the fields of immunology, bacteriology and molecular biology. More particularly, the application relates to methods for obtaining and administering vaccines generated from the investigation of expression libraries constructed from a Chlamydia pneumoniae genome. In particular embodiments, the present application concerns methods and compositions for the vaccination of vertebrate animals against Chlamydia pneumoniae infections, wherein the vaccination of the animal is accomplished through a protein or gene derived from the genes or gene fragments validated as vaccines. In particular embodiments, the animal is a human.

Intracellular bacteria of the genus Chlamydia are important pathogens in both man and vertebrate animals causing blindness in man, sexually transmitted disease and community acquired pneumonia, and most likely act as co-factors in atherosclerotic clot formation in human coronary heart disease.

Specifically, Chlamydia pneumoniae is a major agent of community-acquired respiratory infection and pneumonia. In addition, Chlamydia pneumoniae is strongly associated with atherosclerotic coronary heart disease in developed countries, and is thought to be involved in the pathogenesis of asthma. These public health concerns indicate a requirement for control of such infections.

While antibiotics can be successfully used for the treatment of acute pulmonary infection caused by Chlamydia pneumoniae, once infection and pathology are established, antibiotic treatment has little effect on the outcome of chlamydial diseases. For instance, in large-scale field trials, antibiotic treatment did not influence atherosclerosis that had been associated with increased antibody levels against Chlamydia pneumoniae and the presence of the agent in lesions (Hammerschlag 2003).

As an alternative to a whole pathogen vaccine, recent trends in vaccine development have turned to component or subunit vaccine compositions. Such vaccines are far safer and more consistently manufactured, but have often shown reduced efficacy relative to live or inactivated pathogen vaccines. This has been attributed to reduced complexity and inefficient adjuvants; however another consideration is that the best antigens are rarely if ever established for a vaccine. A solution to this antigen discovery problem is expression library immunization. ELI is a recombinant DNA pooling strategy that enables to assay the full repertoire of genome-encoded components of a pathogen for protective antigens using genetic immunization (GI).

Since the original demonstration of ELI by intramuscular injection of genetic vaccine constructs for protection against Mycoplasma pulmonis pneumonia in mice, a number of methods have been used to deliver genes into a host to raise immune responses against the encoded product. The most commonly used ones have been injection into intramuscular (IM) or intradermal (ID) sites and DNA-coated particle delivery into skin epidermis with a gene gun (Barry et al 2004). In an ELI screen, the whole genome of a pathogen is reconstructed as gene fragments (Barry et al 1995). This library of fragments is manipulated into mammalian expression constructs, partitioned into sublibrary pools, and then used as inocula for test animals. Following pathogen exposure, vaccine utility is evaluated by the single criterion of disease protection (Stemke-Hale et al 2005). Another technology has been developed to speed construction and improve the quality of expression libraries. Linear expression elements (LEEs) are recombinant-DNA constructs that are built wholly in vitro. Namely, there is no amplification or propagation step that uses a live system such as bacterial cloning. LEEs are built by generating an open reading frame (ORF) by PCR, gene assembly, or some other in vitro DNA construction method, and then covalently or non-covalently attaching gene control elements such a promoter and terminator (Sykes et al 1999). The desired recombinant expression vector is constructed completely in vitro and ready to deliver directly in vivo.

The complete 1,230 kb genome sequence of the CDC/CWL-029 strain of Chlamydia pneumoniae has been published by Kalman et al (Nat. Genetics 1999). Using bioinformatics approaches, this knowledge allows identification of all putative ORFs for production of LEE vaccine constructs. Thus, all ORFs can be screened for protective candidate antigens for use in a vaccine against Chlamydia pneumoniae. This approach has been used for testing all Chlamydia pneumoniae genes, and highly protective vaccine candidate genes have been located.

Accordingly, the present application relates to antigens and nucleic acids encoding such antigens obtainable by screening a Chlamydia pneumoniae genome. In more specific aspects, the application relates to methods of isolating protective antigens and nucleic acids and to methods of using such isolated antigens for producing immune responses. The ability of an antigen to produce an immune response may be employed in vaccination or antibody preparation techniques.

In some embodiments, the application relates to isolated polynucleotides having a region that comprises a sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, or SEQ ID NO:21 a complement of any of these sequences, or fragments thereof, or sequences closely related to these sequences. In some more specific embodiments, the application relates to such polynucleotides comprising a region having a sequence comprising at least 17, 20, 25, 30, 35, 40, 45, 50, 60, 70, 80, 90, 100, 125, 150, 200, or more contiguous nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, or SEQ ID NO:21 or its complement. Of course, such polynucleotides may comprise a region having all nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, or SEQ ID NO:21 or its complement.

In another aspect, the application relates to polypeptides having sequences of SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, or SEQ ID NO:20 or fragments thereof, or sequences closely related to these sequences. The application also relates to methods of producing such polypeptides using recombinant methods, for example, using the polynucleotides described above.

The application relates to antibodies against Chlamydia pneumoniae antigens, including those directed against an antigen having polypeptide sequences of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or SEQ ID NO:22 or an antigenic fragment thereof, or sequences closely related to these sequences. The antibodies may be polyclonal or monoclonal and produced by methods known in the art.

The present application contemplates vaccines comprising: (a) a pharmaceutically acceptable carrier, and (b) at least one polynucleotide having a Chlamydia pneumoniae sequence. In one embodiment, the at least one polynucleotide may be isolated from a Chlamydia pneumoniae genomic DNA expression library but it need not be. As discussed below, the polynucleotides need not be of natural origin, or to encode an antigen that is precisely a naturally occurring Chlamydia pneumoniae antigen. It is anticipated that polynucleotides and antigens within the scope of this application may be synthetic and/or engineered to mimic, or improve upon, naturally occurring polynucleotides and still be useful in the invention.

In some embodiments, the at least one polynucleotide has a sequence isolated from Chlamydia pneumoniae, for example, a sequence of SEQ ID NO:1, SEQ ID NO:3, SEQ ID NO:6, SEQ ID NO:9, SEQ ID NO:11, SEQ ID NO:13, SEQ ID NO:15, SEQ ID NO:17, SEQ ID NO:19, or SEQ ID NO:21, or fragment thereof, or sequences closely related to these sequences. In more specific such embodiments, the at least one polynucleotide has a sequence of SEQ ID NO:3, SEQ ID NO:5, SEQ ID NO:9, SEQ ID NO:11, SEQ ID NO:15, or SEQ ID NO:19, or fragment thereof, or sequences closely related to these sequences. In even more specific embodiments, the at least one polynucleotide has a sequence of SEQ ID NO:5 or SEQ ID NO:3.

In some embodiments, the polynucleotide encodes an antigen having a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or SEQ ID NO:22, or antigenic fragment thereof, or sequences closely related to these sequences.

In several embodiments, the polynucleotide is comprised in a genetic immunization vector. Such a vector may, but need not, comprise a gene encoding a mouse ubiquitin fusion polypeptide. The vector, in some preferred embodiments, will comprise a promoter operable in eukaryotic cells, for example, but not limited to a CMV promoter. Such promoters are well known to those of skill in the art. In some embodiments, the polynucleotide is comprised in a viral expression vector, for example, but not limited to, a vector selected from the group consisting of adenovirus, adeno-associated virus, retrovirus and herpes-simplex virus.

The vaccines of the application may comprise multiple polynucleotide sequences. In some embodiments, the vaccine will comprise at least a first polynucleotide having a Chlamydia pneumoniae sequence and a second polynucleotide having a Chlamydia pneumoniae sequence, wherein the first polynucleotide and the second polynucleotide have different sequences. In some more specific embodiments, the first polynucleotide may have a sequence of SEQ ID NO:4, or SEQ ID NO:6.

The present application also involves vaccines comprising: (a) a pharmaceutically acceptable carrier; and (b) at least one Chlamydia pneumoniae antigen. In several embodiments, the at least one Chlamydia pneumoniae antigen has a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or SEQ ID 5 NO:22 or antigenic fragment thereof, or sequences closely related to these sequences. In some specific embodiments, the at least one Chlamydia pneumoniae antigen has a sequence of SEQ ID NO:4, SEQ ID NO:6, SEQ ID NO:10, SEQ ID NO:12, SEQ ID NO:16, or SEQ ID NO:20, or an antigenic fragment thereof, or sequences closely related to these sequences. In even more specific embodiments, the at least one Chlamydia pneumoniae antigen has a sequence of SEQ ID NO:4, or SEQ ID NO:6.

The present application also relates to methods of immunizing an animal comprising providing to the animal at least one Chlamydia pneumoniae antigen, or antigenic fragment thereof, in an amount effective to induce an immune response. In further embodiments, the Chlamydia pneumoniae antigens are comprised of SEQ ID NO:4, SEQ ID NO:6, SEQ ID NO:10, SEQ ID NO:12, SEQ ID NO:16, or SEQ ID NO:20. As discussed above, and described in detail below, the Chlamydia pneumoniae antigens useful in the invention need not be native antigens. Rather, these antigens may have sequences that have been modified in any number of ways known to those of skill in the art, so long as they result in or aid in an antigenic response. In one embodiment, the animal is a human.

In some embodiments of the present application, the provision of the at least one Chlamydia pneumoniae antigen comprises: (a) preparing a cloned expression library from fragmented genomic DNA, cDNA or sequenced genes of Chlamydia pneumoniae; (b) screening the cloned expression library to identify highly protective genes; (c) administering at least one clone of the identified highly protective genes in a pharmaceutically acceptable carrier into an animal; and (d) expressing at least one Chlamydia pneumoniae antigen in the animal. The highly protective genes may comprise at least one or more polynucleotides having a sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, OR SEQ ID NO:17, SEQ ID NO:19, SEQ ID NO:21 or fragment thereof, or sequences closely related to these sequences. The expression library may be cloned in a genetic immunization vector, such vectors and other suitable vectors being well known in the art. The vector may comprise a gene encoding a mouse ubiquitin fusion polypeptide designed to link the expression library polynucleotides to the ubiquitin gene. The vector may comprise a promoter operable in eukaryotic cells, for example a CMV promoter, or any other suitable promoter. An acceptable vector is described in FIG. 1 and the associated text. In such methods, the polynucleotide may be administered by a intramuscular injection or epidermal injection. The polynucleotide may likewise be administered by intravenous, subcutaneous, intralesional, intraperitoneal, oral or inhaled routes of administration. In some specific, exemplary embodiments, the administration may be via intramuscular injection of at least 1.0 μg to 200 μg of the polynucleotide. In other exemplary embodiments, administration may be epidermal injection of at least 0.01 μg to 5.0 μg of the polynucleotide. In some cases, a second administration, for example, an intramuscular injection and/or epidermal injection, may administered at least about three weeks after the first administration. In these methods, the polynucleotide may be, but need not be, cloned into a viral expression vector, for example, a viral expression vector selected from the group consisting of adenovirus, herpes-simple virus, retrovirus and adeno-associated virus. The polynucleotide may also be administered in any other method disclosed herein or known to those of skill in the art.

In some embodiments, the provision of the Chlamydia pneumoniae antigen(s) may comprise: (a) preparing a pharmaceutical composition comprising at least one polynucleotide encoding a Chlamydia pneumoniae antigen or fragment thereof; (b) administering one or more prepared antigen or antigen fragment in a pharmaceutically acceptable carrier into an animal; and (c) expressing one or more Chlamydia pneumoniae antigens in the animal. The one or more polynucleotides can be comprised in one or more expression vectors, as described above and elsewhere in this specification.

Alternatively, the provision of the Chlamydia pneumoniae antigen(s) may comprise: (a) preparing a pharmaceutical composition of at least one Chlamydia pneumoniae antigen or an antigenic fragment thereof; and (b) administering the at least one antigen or fragment into an animal. The antigen(s) may be administered by a first intramuscular injection, intravenous injection, parenteral injection, epidermal injection, inhalation or oral route.

In the embodiments of the application, the animal is a mammal. In some cases the mammal is a bovine, in others, the mammal is a human.

In some embodiments, these methods may induce an immune response against Chlamydia pneumoniae. Alternatively, these methods may be practiced in order to induce an immune response against a Chlamydia species other than Chlamydia pneumoniae, for example, but not limited to, Chlamydia psittaci. Chlamydia trachomatis, and/or Chlamydia pecorum. In some embodiments, these methods may be employed to induce an immune response against a non-Chlamydia infection or other disease.

Thus, the present application is, in one embodiment, directed to a method of immunizing comprising the step of administering a Chlamydia pneumoniae antigen to an animal in an amount effective to induce an immune response against Chlamydia pneumoniae, wherein the antigen comprises the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or SEQ ID NO:22. In one embodiment, the method of immunizing also comprises administering a second Chlamydia pneumoniae antigen in an amount effective to induce an immune response against Chlamydia pneumoniae, wherein the second antigen is distinct from the first antigen and comprises an amino acid sequence selected from SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or SEQ ID NO:22. In one embodiment, the antigen is administered in a pharmaceutically acceptable carrier. In one embodiment, the animal is a human.

This specification discusses methods of obtaining polynucleotide sequences effective for generating an immune response against Chlamydia pneumonia by: (a) preparing a cloned expression library from fragmented genomic DNA of the genus Chlamydia; (b) administering one or more clones of the library in a pharmaceutically acceptable carrier into the animal in an amount effective to induce an immune response; and (c) selecting from the library the polynucleotide sequences that induce an immune response, wherein the immune response in the animal is protective against Chlamydia pneumoniae infection. Such methods may further comprise testing the animal for immune resistance against a Chlamydia pneumoniae bacterial infection by challenging the animal with Chlamydia pneumoniae. In some cases, the genomic DNA has been fragmented physically or by restriction enzymes, for example, but not limited to, fragments that average, about 200-1000 base pairs in length. In some cases, each clone in the library may comprise a gene encoding a mouse ubiquitin fusion polypeptide designed to link the expression library polynucleotides to the ubiquitin gene, but this is not required in all cases. In some cases, the library may comprise about 1×10³ to about 1×10⁶ clones; in more specific cases, the library could have 1×10⁵ clones. In some preferred methods, about 0.01 μg to about 200 μg of DNA, from the clones is administered into the animal. In some situations the genomic DNA, cDNA or sequenced gene is introduced by intramuscular injection or epidermal injection. In some versions of these protocols, the cloned expression library further comprises a promoter operably linked to the DNA that permits expression in a vertebrate animal cell.

The application also discloses methods of preparing antigens that confer protection against infection in an animal comprising the steps of: (a) preparing a cloned expression library from fragmented genomic DNA of the Chlamydia pneumoniae genome; (b) administering one or more clones of the library in a pharmaceutically acceptable carrier into the animal in an amount effective to induce an immune response; (c) selecting from the library the polynucleotide sequences that induce an immune response and expressing the polynucleotide sequences in cell culture; and (d) purifying the polypeptide(s) expressed in the cell culture. Often, these methods further comprise testing “the animal for immune resistance against infection by challenging the animal with Chlamydia pneumoniae or other pathogens.

The application relates to methods of preparing antibodies against a Chlamydia pneumoniae antigen comprising the steps of (a) selecting a Chlamydia pneumoniae antigen that confers immune resistance against Chlamydia pneumoniae infection when challenged with Chlamydia pneumoniae; (b) generating an immune response in a vertebrate animal with the antigen identified in step (a); and (c) obtaining antibodies produced in the animal.

The application also relates to methods of assaying for the presence of Chlamydia pneumoniae infection in a vertebrate animal comprising: (a) obtaining an antibody directed against a Chlamydia pneumoniae antigen; (b) obtaining a sample from the animal; (c) admixing the antibody with the sample; and (d) assaying the sample for antigen-antibody binding, wherein the antigen-antibody binding indicates Chlamydia pneumoniae infection in the animal. In some cases, the antibody directed against the antigen is further defined as a polyclonal antibody. In others, the antibody directed against the antigen is further defined as a monoclonal antibody. In some embodiments, the Chlamydia pneumoniae antigen has a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, or fragment thereof, or sequences closely related to these sequences. The assaying the sample for antigen-antibody binding may be by precipitation reaction, radioimmunoassay, ELISA, Western blot, immunofluorescence, or any other method known to those of skill in the art.

The application also relates to kits for assaying a Chlamydia pneumoniae infection comprising, in a suitable container: (a) a pharmaceutically acceptable carrier; and (b) an antibody directed against a Chlamydia pneumoniae antigen, wherein the antibody binds to a Chlamydia pneumoniae antigen having the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38.

The application further relates to methods of assaying for the presence of a Chlamydia pneumoniae infection in an animal comprising: (a) obtaining an oligonucleotide probe comprising a sequence comprised within one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, or SEQ ID NO:37, or a complement thereof; and (b) employing the probe in a PCR or other detection protocol.

As used herein in the specification, “a” or “an” may mean one or more. As used herein, when used in conjunction with the word “comprising,” the words “a” or “an” may mean one or more than one. As used herein “another” may mean at least a second or more.

As used herein, “plurality” means more than one. In certain specific aspects, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 400, 500, 750, 1,000, 2,000, 3,000, 4,000, 5,000, 7,500, 10,000, 15,000, 20,000, 30,000, 40,000, 50,000, 60,000, 70,000, 80,000, 90,000, 100,000, 125,000, 150,000, 200,000 or more, and any integer derivable therein, and any range derivable therein.

As used herein, “any integer derivable therein” means a integer between the numbers described in the specification, and “any range derivable therein” means any range selected from such numbers or integers.

As used herein, a “fragment” refers to a sequence having or having at least 5, 10, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 890, 900, 910, 920, 930, 940, 950, 960, 970, 980, 990, 1000 or more contiguous residues of the recited SEQ ID NOS, but less than the full-length of the SEQ, ID NOS. It is contemplated that the definition of “fragment” can be applied to amino acid and nucleic acid fragments.

As used herein, an “antigenic fragment” refers to a fragment, as defined above, that can elicit an immune response in an animal. The term “animal” may refer to any animal, including vertebrate animals and particularly including humans.

Reference to a sequence in an organism, such as a “Chlamydia sequence” refers to a segment of contiguous residues that is unique to that organism or that constitutes a fragment (or full-length region(s)) found in that organism (either amino acid or nucleic acid).

### BRIEF DESCRIPTION OF THE DRAWINGS

The following drawings form part of the present specification and are included to further demonstrate certain aspects of the present application. The application may be better understood by reference to one or more of these drawings in combination with the detailed description of specific embodiments presented herein.

FIG. 1. Scheme for Expression Library Immunization.

FIG. 2. Recombinant mammalian expression vector used in Round 3 immunization experiments. Vector pCMVi-UB contains individual bacterial genes under control of the eukaryotic modified cytomegalovirus immediate-early promoter enhanced by a chimeric intron (CMVi). A eukaryotic expression cassette was cloned into a generic bacterial plasmid containing pBR322, f1 and SV40 origins of replication and an ampicillin resistance gene. The eukaryotic expression cassette contains a mouse ubiquitin encoding sequence under control of the CMVi promoter and flanked by a multicloning site and a human growth hormone terminator. The bacterial protein encoding sequences were cloned into unique BglII and HindIII restriction site in a manner that ensured continuity of the ubiquitin into a bacterial reading frame. The recombinant cassette expressed a fusion protein comprised of mouse ubiquitin and bacterial protein separated with a linker.

FIG. 3. Evaluation of Chlamydia pneumoniae lung infection in mice over fifteen days. Six week-old female mice of either A/J or C57BL/6 strains received a pre-challenge mock inoculum (naïve) or a low-dose Chlamydia pneumoniae inoculum (5×10⁶ EB; immune), were intranasally challenged 4 weeks later with 1×10⁸ Chlamydia pneumoniae, and sacrificed 2 hours (day 0), 3 days, 10 days, or 15 days after inoculation to calibrate the range of achievable protection, i.e., provide experimental controls. Lung total nucleic acids were extracted, and Chlamydia pneumoniae genomes were determined by Chlamydia pneumoniae 23S rRNA FRET real-time PCR. Data are means (n=10)±95% confidence intervals. Asterisks indicate significant differences between groups (p<0.05, Tukey HSD test). A. Time course of lung weight increase in naïve and Chlamydia pneumoniae-immune A/J mice, and B. in naïve and Chlamydia pneumoniae-immune C57BL/6 mice. C. Time course of Chlamydia pneumoniae lung burdens in A/J mice and D. in C57BL/6 mice. Lung weight increases of immune C57BL/6 mice on days 10 and 15 post inoculation are significantly higher than of A/J mice (p<0.01). Immune A/J mice on day 3 μl have a higher lung weight increase than naïve A/J mice (p=0.008). On days 10 and 15 pi, immune A/J mice, but not C57BL/6 mice, have highly significantly lower lung Chlamydia pneumoniae loads than naïve mice p<0.001).

FIG. 4. Time course of lung transcripts of immune-associated genes in control mice after challenge inoculation. Mice immunized by a previous low-dose inoculation were challenged intranasally with 1×10⁸ Chlamydia pneumoniae, and sacrificed 2 hours (day 0), 3 days, or 10 days after inoculation (n=10). Lung poly(A)⁺ was extracted, and transcripts were quantified by one-step duplex real-time RT-PCR. Data are expressed as numbers of the respective transcripts per 1000 transcripts of the PBGD reference gene (means ±95% confidence intervals). Asterisks indicate significant differences between groups (p<0.05, Tukey HSD test). A. Time course of lung Tim3 transcripts (associated with Th1 immunity). B. Lung GATA3 transcripts (associated with Th2 immunity). C. The transcript ratio of Tim3:GATA3. D. Lung CD45RO transcripts (memory T cell-associated). Immune A/J mice show higher early Tim3 transcripts and Th1 immune bias (Tim3/GATA3), and overall higher memory T cell CD45RO transcripts than C57BL/6 mice (combined CD45RO data; p=0.008).

FIG. 5. Day-10 pi plasma levels of anti-Chlamydia pneumoniae antibody isotypes in A/J mice. Naïve A/J mice and A/J mice immunized by a previous low-dose inoculation were challenged intranasally with 1×10⁸ Chlamydia pneumoniae, and plasma was obtained on day 10 post inoculation. Mouse IgG1 and IgG2a antibodies binding to Chlamydia pneumoniae lysate antigen were determined by chemiluminescent ELISA. Data are expressed in relative light units (rlu) as means (n=20)±95% confidence intervals. Immune animals have highly significant higher IgG2a antibody levels and IgG2a:IgG1 ratio than naïve mice on day 10 after challenge (p<0.001).

FIG. 6. Round-1 ELI screen of the complete Chlamydia pneumoniae genome for protective capacity. The Linear Expression Element (LEE) library of Chlamydia pneumoniae open reading frames (ORFs) was arrayed into 90 pools (30 X—, 30 Y—, and 30 Z) of ˜42 LEE constructs each that were used as inocula for 3 gene gun immunizations in 4-week intervals (n=5 mice/pool). Each test inoculum contained 200 ng of a mixture of ˜42 ORFs and 800 ng of pUC118 carrier DNA. Four weeks after the last immunization, all mice were challenged by intranasal inoculation of 1×10⁸ Chlamydia pneumoniae organisms and sacrificed 10 days later. Positive control, immune mice received a low-dose inoculum of Chlamydia pneumoniae 4 weeks prior to high dose challenge. Immune and naïve groups (n=20) were used to calibrate the range of possible protection. Another set of negative control animals was immunized with a construct expressing an irrelevant (LUC) gene product (n=10). A. Group means of total Chlamydia pneumoniae lung loads (genomes) determined by real-time PCR. The area below the horizontal line corresponds to the area above the protection threshold line in panel B. B. Protective capacity of all test groups. The protection scores are calibrated by a 100% protection score of the immune group and a 0% protection of the naïve group. The area above the horizontal line contains the vaccine pools that were used to select candidate protection ORFs. ORFs were ranked using the sum of protection scores of the ORF's respective XYZ pools three-way intersection approach of pools above the protection threshold. The combined approach selected 46 Chlamydia pneumoniae ORFs for further testing in the individual vaccine candidate screens in rounds 2 and 3.

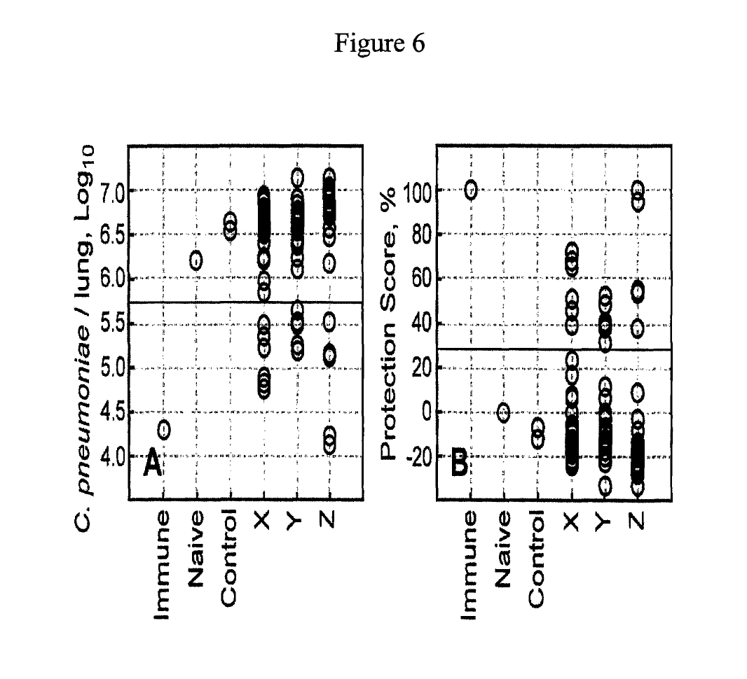

FIG. 7. Disease protection efficacy of final vaccine candidates. After testing of 46 individual candidates in round 2, 12 of these genes were cloned as full-length genes (except ide_ab and Cpn0095_a) into genetic immunization plasmid CMVi-UB and used for vaccination in round 3. Cpn0095_a was not included in the round-3 high-dose challenge. Vaccinated mice (n=10/group) were intranasally challenged with an LD₅₀ of 5×10⁸ Chlamydia pneumoniae elementary bodies. Surviving mice were sacrificed on day 10 post inoculation, lungs were weighed, and the lung weight increase over the average lung weight of unchallenged age-matched female A/J mice was calculated. The lung weight increase is a reliable measure of disease intensity, and high increases reflect severe disease. Lung weight increase data were linearly transformed into protection scores by setting the score for unprotected naïve mice at 0 and for optimally protected live-vaccinated mice at 1. Data are shown as means means ±95% confidence intervals.

FIG. 8. Vaccine protective efficacy of final vaccine candidates for elimination of Chlamydia pneumoniae. For vaccination rounds 2 and 3 of the final vaccine candidate genes, protection scores were calculated based on the logarithm of the total Chlamydia pneumoniae lung load on day 10. Protection score data from round 2 with the use of LEE constructs and from round 3 with plasmid-cloned genes (full-length except for partial genes ide_ab and Cpn0095_a) were pooled and analyzed by one-way ANOVA. Data are shown as means means ±95% confidence intervals (naïve, live vaccine groups n=60; genetic vaccine groups n=13-20).

### DETAILED DESCRIPTION

Chlamydia pneumoniae is a species of chlamydiae bacteria that infects humans and is a major cause of pneumonia. Chlamydia pneumoniae has a complex life cycle and must infect another cell in order to reproduce and thus is classified as an obligate intracellular pathogen. In addition to its role in pneumonia, there is evidence associating Chlamydia pneumoniae with atherosclerosis and with asthma.

Chlamydia pneumoniae is a common cause of pneumonia around the world. Chlamydia pneumoniae is typically acquired by otherwise healthy people and is a form of community-acquired pneumonia. Because treatment and diagnosis are different from historically recognized causes such as Streptococcus pneumoniae, pneumonia caused by Chlamydia pneumoniae is categorized as an “atypical pneumonia.”

Typically, treatment for pneumonia is begun before the causative microorganism is identified. This empiric therapy includes an antibiotic active against the bacteria. The most common type of antibiotic used is a macrolide such as azithromycin or clarithromycin. If testing reveals that Chlamydia pneumoniae is the causative agent, therapy may be switched to doxycycline, which may be slightly more effective against the bacteria. Sometimes a quinolone antibiotic such as levofloxacin may be started empirically. This group is not as effective against Chlamydia pneumoniae. Treatment is typically continued for ten to fourteen days for known infections.

The present application is directed to compositions and methods for the immunization of vertebrate animals, including humans, against infections using nucleic acid sequences and polypeptides elucidated by screening Chlamydia pneumoniae. These compositions and methods will be useful for immunization against Chlamydia pneumoniae infections and other infections and disease states. In particular embodiments, a vaccine composition directed against Chlamydia pneumoniae infections is provided. The vaccine according to the present application comprises Chlamydia pneumoniae genes and polynucleotides identified by the inventors, that confer protective resistance in vertebrate animals to Chlamydia pneumoniae bacterial infections, and other infections. In other embodiments, the application provides methods for immunizing an animal against Chlamydia pneumoniae infections and methods for screening and identifying Chlamydia pneumoniae genes that confer protection against infection.

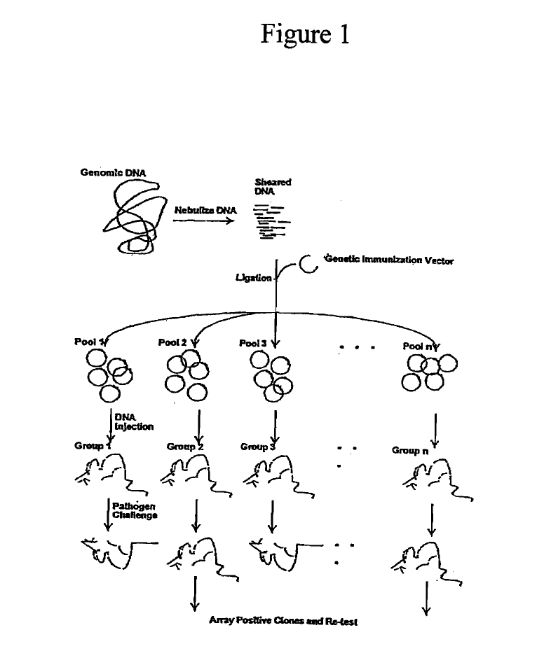

Referring to FIG. 1, in order to identify the unique nucleic acid and polypeptide sequences that confer protection, a library of Chlamydia pneumoniae linear expression elements (LEEs) was constructed. Specifically, all putative open reading frames of the Chlamydia pneumoniae genome were amplified by PCR, and promoter and terminator polynucleotides were attached. These constructs were combined in various pools and used for expression library immunization. Expression library immunization (ELI herein) is well known in the art—U.S. Pat. No. 5,703,057, specifically incorporated herein by reference. The ELI method operates on the assumption, generally accepted by those skilled in the art, that all the potential anti genic determinants of any pathogen are encoded in its genome. The method uses to its advantage the simplicity of genetic immunization to sort through a genome for immunological reagents in an unbiased, systematic fashion.

The preparation of an expression library is performed using the techniques and methods familiar one of skill in the art. The pathogen's genome, may or may not be known or possibly may even have been cloned. Thus one obtains DNA (or cDNA), representing substantially the entire genome or all open reading frames of the pathogen (e.g., Chlamydia pneumoniae) The DNA is broken up, by physical fragmentation or restriction endonuclease, into segments of some length so as to provide a library of about 10⁵ (approximately 18× the genome size) members. Alternatively, LEEs of all PCR-amplified open reading frames are constructed. The library is then tested by inoculating a subject with purified DNA of the library or sub-library and the subject challenged with a pathogen, wherein immune protection of the subject from pathogen challenge indicates a clone that confers a protective immune response against infection.

The present application discloses Chlamydia pneumoniae polynucleotide compositions and methods that induce a protective immune response in vertebrate animals challenged with a Chlamydia pneumoniae bacterial infection. The preparation and purification of antigenic Chlamydia polypeptides, or fragments thereof and antibody preparations directed against Chlamydia antigens, or fragments thereof are described below.

Thus, in certain embodiments, genes or polynucleotides encoding Chlamydia pneumoniae polypeptides or fragments thereof are provided. It is contemplated that in other embodiments, a polynucleotide encoding a Chlamydia pneumoniae polypeptide or polypeptide fragment will be expressed in prokaryotic or eukaryotic cells and the polypeptides purified for use as anti-Chlamydia pneumoniae antigens in the vaccination of vertebrate animals or in generating antibodies immunoreactive with Chlamydia pneumoniae polypeptides (i.e., antigens).

The present application, therefore, discloses polynucleotides encoding antigenic Chlamydia pneumoniae polypeptides capable of inducing a protective immune response in vertebrate animals and for use as an antigen to generate anti-Chlamydia pneumoniae or other pathogen antibodies. In certain instances, it may be desirable to express Chlamydia pneumoniae polynucleotides encoding a particular antigenic Chlamydia pneumoniae polypeptide domain or as a sequence to be used as a vaccine or in generating anti-Chlamydia pneumoniae or other pathogen antibodies. Nucleic acids according to the present application may encode an entire Chlamydia pneumoniae gene, or any other fragment of the Chlamydia pneumoniae sequences set forth herein. Experiments have been conducted to demonstrate the efficiency of both fragments and full length genes in providing a protective immune response. The nucleic acid may be derived from genomic DNA, i.e., cloned or PCR-amplified directly from the genome of a particular organism. In other embodiments, however, the nucleic acid may comprise complementary DNA (cDNA). A protein may be derived from the designated sequences for use in a vaccine or to isolate useful antibodies.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression.

It also is contemplated that a given Chlamydia pneumoniae polynucleotide from a given species may be represented by natural variants that have slightly different nucleic acid sequences but, nonetheless, encode the same polypeptide (see Table 1 below). In addition, it is contemplated that a given Chlamydia polypeptide from a species may be generated using alternate codons that result in a different nucleic acid sequence but encodes the same polypeptide.

As used in this application, the term “a nucleic acid encoding a Chlamydia pneumoniae polynucleotide” refers to a nucleic acid molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | He | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of given Chlamydia pneumoniae gene or polynucleotide. Sequences that are essentially the same as those set forth in a Chlamydia pneumoniae gene or polynucleotide may also be functionally defined as sequences that are capable of hybridizing to a nucleic acid segment containing the complement of a Chlamydia pneumoniae polynucleotide under standard conditions.

Thus, modifications and changes may be made in the structure of a gene and a functional molecule that encodes a protein or polypeptide with desirable characteristics may be obtained. Certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics, these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

The DNA segments of the present application include those encoding biologically functional equivalent Chlamydia pneumoniae proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site directed mutagenesis techniques or may be introduced randomly and screened later for the desired function, as described below.

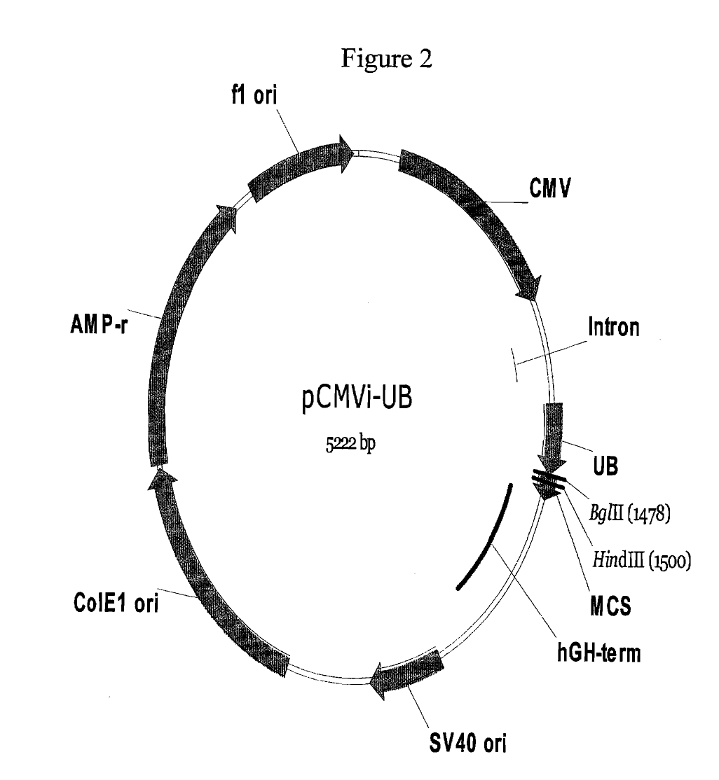

Referring now to FIG. 2, the polynucleotide vaccines of the present application may comprise a genetic immunization vector or a viral expression vector. Genetic immunization vectors are well known in the art, for example, the general approach in these systems is to provide a cell with an expression construct encoding a specific protein, polypeptide or polypeptide fragment to express in the cell. Following delivery of the vector, the protein, polypeptide or polypeptide fragment is synthesized by the transcriptional and translational machinery of the cell and released from the cell into whatever host the vector is provided. The viral expression vector may be an adenovirus vector, and adeno-associated virus vector, a retrovirus vector or a Herpes-Simplex viral vector. Some acceptable vectors are described in U.S. Pat. Nos. 5,670,488; 5,739,018; 5,824,544; 5,851,826; 5,858,744; 5,879,934; 5,932,210; 5,955,331, which are hereby incorporated by reference. Other methods of polynucleotide delivery are also contemplated, including, but not limited to non-viral polynucleotide delivery through particle bombardment or receptor mediated gene targeting vehicles.

Naturally, the present application also encompasses nucleotide segments that are complementary, or essentially complementary to identified sequences of a Chlamydia pneumoniae polynucleotide. Nucleic acid sequences that are “complementary” are those that are capable of base-pairing according to the standard Watson-Crick complementary rules and are well known in the art. As used herein, the term “complementary sequences” means nucleic acid sequences that are substantially complementary, as may be assessed by the same nucleotide comparison set forth above, or as defined as being capable of hybridizing to the nucleic acid segment of a Chlamydia pneumoniae polynucleotide under relatively stringent conditions well known in the art, for example, using site-specific mutagenesis. Such sequences may encode the entire Chlamydia pneumoniae polypeptide or functional or non-functional fragments thereof.

For the purposes of the present application, a Chlamydia pneumoniae polypeptide used as an antigen may be a naturally-occurring Chlamydia pneumoniae polypeptide that has been extracted using protein extraction techniques well known to those of skill in the art, such as ELI, and prepared in a pharmaceutically acceptable carrier for the vaccination of an animal against Chlamydia pneumoniae infection. In alternative embodiments, the Chlamydia pneumoniae polypeptide or antigen may be a synthetic peptide. In still other embodiments, the peptide may be a recombinant peptide produced through molecular engineering techniques.

Chlamydia pneumoniae genes or their corresponding cDNA identified in the present application can be inserted into an appropriate cloning vehicle for the production of Chlamydia pneumoniae polypeptides as antigens. The transcription of a polypeptide sequence from a polynucleotide sequence is well known in the art.

In addition, sequence variants of the polypeptide can be prepared. The variants may, for instance, be minor sequence variants of the polypeptide that arise due to natural variation within the population, or they may be homologues found in other species. They also may be sequences that do not occur naturally, but that are sufficiently similar that they function similarly and/or elicit an immune response that cross-reacts with natural forms of the polypeptide. Sequence variants can be prepared by standard methods of site-directed mutagenesis well known in the art.

Another synthetic or recombinant variation of a Chlamydia-antigen is a polyepitopic moiety comprising repeats of epitopic determinants found naturally on Chlamydia pneumoniae proteins. Such synthetic polyepitopic proteins can be made up of several homomeric repeats of anyone Chlamydia pneumoniae protein epitope; or can comprise of two or more heteromeric epitopes expressed on one or several Chlamydia pneumoniae protein epitopes.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species, such as Chlamydia psittaci or Chlamydia trachomatis. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide may be identified by an empirical approach in which portions of the gene encoding the polypeptide are expressed in a recombinant host, and the resulting proteins tested for their ability to elicit an immune response. For example, the polymerase chain reaction (PCR) can be used to prepare a range of cDNAs encoding peptides lacking successively longer fragments of the C-terminus of the protein. The immunogenic activity of each of these peptides then identifies those fragments or domains of the polypeptide that are essential for this activity. Further experiments in which only a small number of amino acids are removed or added at each iteration then allows the location of other antigenic determinants of the polypeptide. Thus, the polymerase chain reaction, a technique for amplifying a specific segment of DNA via multiple cycles of denaturation-renaturation, using a thermostable DNA polymerase, deoxyribonucleotides and primer sequences is contemplated.

Another embodiment for the preparation of the polypeptides according to the application is the use of peptide mimetics. Mimetics are peptide-containing molecules that mimic elements of protein secondary structure. Because many proteins exert their biological activity via relatively small regions of their folded surfaces, their actions can be reproduced by much smaller designer (mimetic) molecules that retain the bioactive surfaces and have potentially improved pharmacokinetic/dynamic properties.

The underlying rationale behind the use of peptide mimetics is that the peptide backbone of proteins exists chiefly to orient amino acid side chains in such a way as to facilitate molecular interactions, such as those of antibody and antigen. However, unlike proteins, peptides often lack well defined three dimensional structure in aqueous solution and tend to be conformationally mobile. Progress has been made with the use of molecular constraints to stabilize the bioactive conformations. By affixing or incorporating templates that fix secondary and tertiary structures of small peptides, synthetic molecules (protein surface mimetics) can be devised to mimic the localized elements of protein structure that constitute bioactive surfaces. Methods for predicting, preparing, modifying, and screening mimetic peptides are described in U.S. Pat. No. 5,933,819 and U.S. Pat. No. 5,869,451 (each specifically incorporated herein by reference). It is contemplated in the present application, that peptide mimetics will be useful in screening modulators of an immune response.

In certain embodiments, the synthesis of a Chlamydia pneumoniae peptide fragment is considered. The peptides of the application can be synthesized in solution or on a solid support in accordance with conventional techniques. Various automatic synthesizers are commercially available and can be used in accordance with well known protocols. Alternatively, recombinant DNA technology may be employed wherein a nucleotide sequence which encodes a peptide of the application is inserted into an expression vector, transformed or transfected into an appropriate host cell and cultivated under conditions suitable for expression.

The present application contemplates the purification, and in particular embodiments, the substantial purification, of Chlamydia pneumoniae polypeptides. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “− fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE. It will, therefore, be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The present application provides antibody compositions that are immunoreactive with a Chlamydia pneumoniae polypeptide of the present application, or any portion thereof.

An antibody can be a polyclonal or a monoclonal antibody. An antibody may also be monovalent or bivalent. A prototype antibody is an immunoglobulin composed by four polypeptide chains, two heavy and two light chains, held together by disulfide bonds. Each pair of heavy and light chains forms an antigen binding site, also defined as complementarity-determining region (CDR). Therefore, the prototype antibody has two CDRs, can bind two antigens, and because of this feature is defined bivalent. The prototype antibody can be split by a variety of biological or chemical means. Each half of the antibody can only bind one antigen and, therefore, is defined monovalent. Means for preparing and characterizing antibodies are well known in the art.

Peptides corresponding to one or more antigenic determinants of a Chlamydia polypeptide of the present application also can be prepared. Such peptides should generally be at least five or six amino acid residues in length, will preferably be about 10, 15, 20, 25 or about 30 amino acid residues in length, and may contain up to about 35-50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

The identification and preparation of epitopes from primary amino acid sequences on the basis of hydrophilicity is taught in U.S. Pat. No. 4,554,101 (Hopp), incorporated herein by reference. Through the methods disclosed in Hopp, one of skill in the art would be able to identify epitopes from within an amino acid sequence such as a Chlamydia pneumoniae polypeptide sequence. Predictable computer simulations and software well known in the art may be used to supplement and assist in predicting antigenic regions, such as PEPPLOT® available from the University of Wisconsin Biotechnology Center in Madison, Wis. or MACVECTOR available from IBI of New Haven, Conn.

In further embodiments, major antigenic determinants of a Chlamydia pneumoniae polypeptide may be identified by an empirical approach in which portions of the gene encoding the polypeptide are expressed in a recombinant host, and the resulting proteins tested for their ability to elicit an immune response. For example, PCR can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTS system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using peR cloning methodology.

The use of such small peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

The present application provides monoclonal antibody compositions that are immunoreactive with a Chlamydia pneumoniae polypeptide. As detailed above, in addition to antibodies generated against a full length Chlamydia polypeptide, antibodies also may be generated in response to smaller constructs comprising epitopic core regions, including wild-type and mutant epitopes. In other embodiments of the application, the use of anti-Chlamydia pneumoniae single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred.

However, “humanized” Chlamydia pneumoniae antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-Chlamydia pneumoniae humanized or chimeric antibody can be genetically engineered to comprise a Chlamydia pneumoniae antigen binding site of a given of molecular weight and biological lifetime, as long as the antibody retains its Chlamydia pneumoniae antigen binding site.

The term “antibody” is used to refer to any antibody-like molecule that has an antigen binding region, and includes antibody fragments such as Fab′, Fab, F(ab′h, single domain antibodies (DABs), Fv, scFv (single chain Fv), chimeras and the like. Methods and techniques of producing the above antibody-based constructs and fragments are well known in the art (U.S. Pat. No. 5,889,157; U.S. Pat. No. 5,821,333; U.S. Pat. No. 5,888,773, each specifically incorporated herein by reference).

U.S. Pat. No. 5,889,157 describes a humanized B3 scFv antibody preparation. The B3 scFv is encoded from a recombinant, fused DNA molecule, that comprises a DNA sequence encoding humanized Fv heavy and light chain regions of a B3 antibody and a DNA sequence that encodes an effector molecule. The effector molecule can be any agent having a particular biological activity which is to be directed to a particular target cell or molecule. Described in U.S. Pat. No. 5,888,773, is the preparation of scFv antibodies produced in eukaryotic cells, wherein the scFv antibodies are secreted from the eukaryotic cells into the cell culture medium and retain their biological activity. It is contemplated that similar methods for preparing multi-functional anti-Chlamydia pneumoniae fusion proteins, as described above, may be utilized in the present application.

Means for preparing and characterizing antibodies also are well known in the art. The methods for generating monoclonal antibodies (mAbs) generally begin along the same lines as those for preparing polyclonal antibodies. Briefly, a polyclonal antibody is prepared by immunizing an animal with an immunogenic Chlamydia pneumoniae composition in accordance with the present application and collecting antisera from that immunized animal.

A wide range of animal species can be used for the production of antisera. Typically, the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary, therefore, to boost the host immune system, as may be achieved by coupling a peptide or polypeptide immunogen to a carrier. Exemplary and preferred carriers are keyhold limpet hemocyanin (KLH) and bovine serium albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, Quil-A, a plant saponin, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (SmithKline Beecham, Pa.); low-dose Cyclophosphamide (CYP; 300 mg/m²) (Johnson & Johnson, Mead, N.J.), cytokines such as y-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate mAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified Chlamydia pneumoniae polypeptide, peptide or domain, be it a wild-type or mutant composition. The immunizing composition is administered in a manner effective to stimulate antibody producing cells.

The methods for generating monoclonal antibodies (mAbs) generally begin along the same lines as those for preparing polyclonal antibodies. Rodents such as mice and rate are contemplated in some embodiments; however, the use of rabbit, sheep or frog cells also is possible.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals, or the gene encoding the protein of interest can be directly injected.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes.

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. HAT medium, a growth medium containing hypoxanthine, aminopterin and thymidine, is well known in the art as a medium for selection of hybrid cells. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas then would be serially diluted and cloned into individual antibody-producing cell lines, which clones can then be propagated indefinitely to provide mAbs. The cell lines may be exploited for mAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide mAbs in high concentration. Second, the individual cell lines could be cultured in vitro, where the mAbs are naturally secreted into the culture medium from which they can be readily obtained in high concentrations.

mAbs produced by either means may be further purified, if desired, using filtration, centrifugation and various chromatographic methods such as HPLC or affinity chromatography. Fragments of the monoclonal antibodies of the application can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present application can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present application can be synthesized using an automated peptide synthesizer, or by expression of full-length gene or of gene fragments in, for example, E. coli.

Compositions of the present application comprise an effective amount of a purified Chlamydia pneumoniae polynucleotide and/or a purified Chlamydia pneumoniae a protein, polypeptide, peptide, epitopic core region, and the like, dissolved and/or dispersed in a pharmaceutically acceptable carrier and/or aqueous medium. Aqueous compositions of gene therapy vectors expressing any of the foregoing are also contemplated.

The phrases “pharmaceutically and/or pharmacologically acceptable” refer to molecular entities and/or compositions that do not produce an adverse, allergic and/or other untoward reaction when administered to an animal.

As used herein, “pharmaceutically acceptable carrier” includes any and/or all solvents, dispersion media, coatings, antibacterial and/or antifungal agents, isotonic and/or absorption delaying agents and the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For animal and more particularly human administration, preparations should meet sterility, pyrogenicity, general safety and/or purity standards as required by FDA Office of Biologics standards.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes, or formulated for oral or inhaled delivery. The preparation of an aqueous composition that contains an effective amount of purified Chlamydia pneumoniae polynucleotide or polypeptide agent as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

A Chlamydia pneumoniae polynucleotide or polypeptide of the present application can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion. Some variation in dosage will necessarily occur depending on the condition of the subject being treated. The person responsible for administration will, in any event, determine the appropriate dose for the individual subject.

A Chlamydia polynucleotide or protein-derived peptides and/or agents may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present application. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

Therapeutic kits of the present application are kits comprising a Chlamydia pneumoniae polynucleotide or polypeptide. Such kits will generally contain, in a suitable container, a pharmaceutically acceptable formulation of a Chlamydia pneumoniae polynucleotide or polypeptide or vector expressing any of the foregoing in a pharmaceutically acceptable formulation. The kit may have a single container, and/or it may have a distinct container for each compound.

When the components of the kit are provided in one and/or more liquid solutions, the liquid solution is an aqueous solution, with a sterile aqueous solution being particularly preferred. The Chlamydia pneumoniae polynucleotide or polypeptide compositions may also be formulated into a syringeable composition. In which case, the container may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container.

The container will generally include at least one vial, test tube, flask, bottle, syringe and/or other container, into which the Chlamydia pneumoniae polynucleotide or polypeptide formulation are placed, preferably, suitably allocated. The kits may also comprise a second container for containing a sterile, pharmaceutically acceptable buffer and/or other diluent.

The kits of the present application will also typically include a means for containing the vials in close confinement for commercial sale, such as, e.g., injection and/or blowmolded plastic containers into which the desired vials are retained.

Irrespective of the number and/or type of containers, the kits of the application may also comprise, and/or be packaged with, an instrument for assisting with the injection/administration and/or placement of the ultimate Chlamydia pneumoniae polynucleotide or polypeptide within the body of an animal. Such an instrument may be a syringe, pipette, forceps, and/or any such medically approved delivery vehicle.

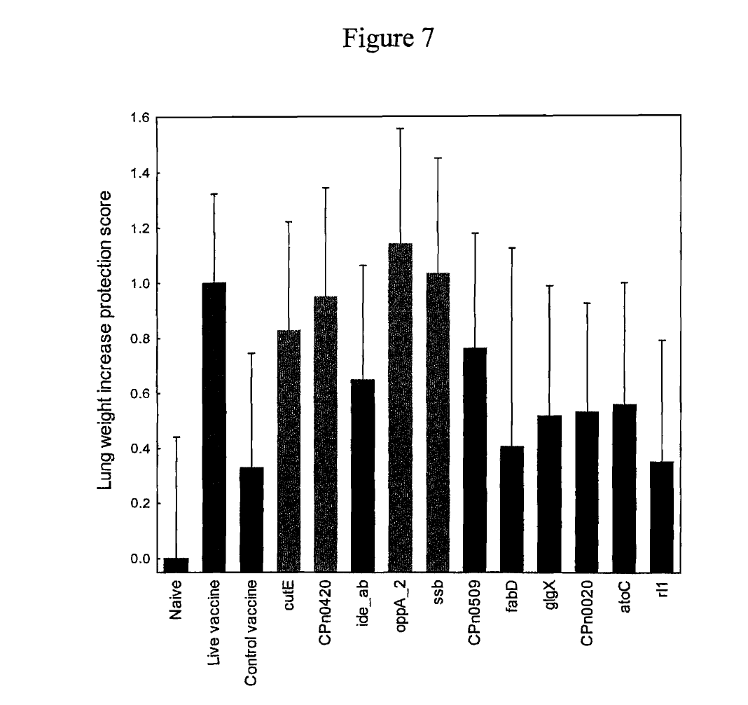

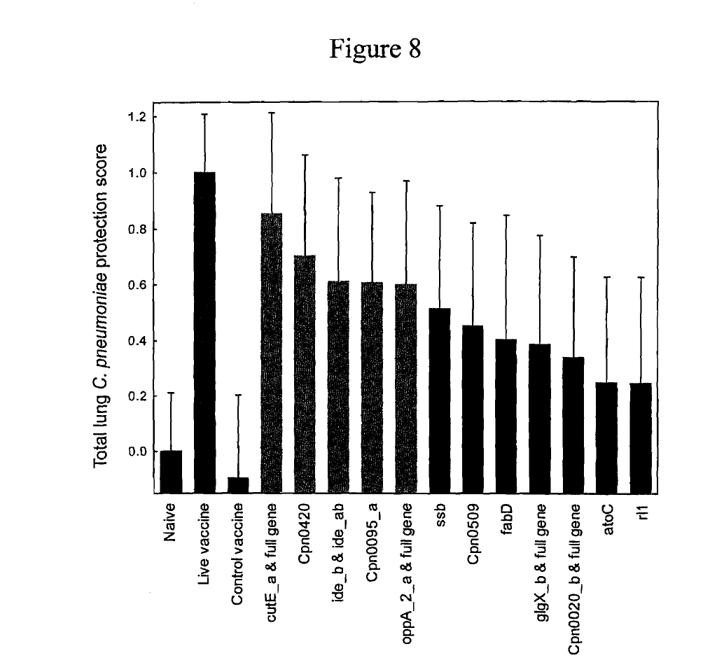

The present application discloses several polynucleotide and polypeptide sequences that code for proteins that provide a protective response against Chlamydia pneumoniae infection. FIGS. 7 and 8 summarize the protective genes, and the following table (Table 2) correlates the protective genes with the provided sequence identification numbers (SEQ ID NO) that identify the particular polynucleotide and polypeptide sequences in the sequence listing appended hereto.

TABLE 2

| SEQ ID NO | GENE/GENE FRAGMENT | TYPE OF SEQUENCE |
|---|---|---|
| 1 | cut E | polynucleotide |
| 2 | cut E | polypeptide |
| 3 | cut E_a (fragment) | polynucleotide |
| 4 | cut E_a (fragment) | polypeptide |
| 5 | Cpn0420 | polynucleotide |
| 6 | Cpn0420 | polypeptide |
| 7 | Ide | polynucleotide |
| 8 | Ide | polypeptide |
| 9 | ide_b (fragment) | polynucleotide |
| 10 | ide_b (fragment) | polypeptide |
| 11 | ide_ab (fragment) | polynucleotide |
| 12 | ide_ab (fragment) | polypeptide |
| 13 | Cpn0095 | polynucleotide |
| 14 | Cpn0095 | polypeptide |
| 15 | Cpn0095_a (fragment) | polynucleotide |
| 16 | Cpn0095_a (fragment) | polypeptide |
| 17 | oppA_2 | polynucleotide |
| 18 | oppA_2 | polypeptide |
| 19 | oppA_2_a (fragment) | polynucleotide |
| 20 | oppA_2_a (fragment) | polypeptide |
| 21 | Ssb | polynucleotide |
| 22 | Ssb | polypeptide |
| 23 | Cpn0509 | polynucleotide |
| 24 | Cpn0509 | polypeptide |
| 25 | fabD | polynucleotide |
| 26 | fabD | polypeptide |
| 27 | glgX | polynucleotide |
| 28 | glgX | polypeptide |
| 29 | glgX_b (fragment) | polynucleotide |
| 30 | glgX_b (fragment) | polypeptide |
| 31 | Cpn0020 | polynucleotide |
| 32 | Cpn0020 | polypeptide |
| 33 | Cpn0020_b | polynucleotide |
| 34 | Cpn0020_b | polypeptide |
| 35 | atoC | polynucleotide |
| 36 | atoC | polypeptide |
| 37 | rl1 | polynucleotide |
| 38 | rl1 | polypeptide |

One of ordinary skill in the art will understand that Table 2 demonstrates first a polynucleotide (e.g., DNA) sequence for a gene or gene fragment and then the corresponding polypeptide (e.g., amino acid) sequence for the same gene or gene fragment. Thus, for example, SEQ ID NO:1 is a polynucleotide sequence corresponding to the polypeptide sequence of SEQ ID NO:2. Both SEQ ID NO:1 and SEQ ID NO:2 code for the same final protein. One of ordinary skill in the art will also understand that fragments of a gene, e.g., cutE_a, is contained within the full length gene, e.g., cutE. Thus, for example, SEQ ID NO:3 is contained in SEQ ID NO:1, and SEQ ID NO:4 is contained in SEQ ID NO:2.

Since the identified sequences demonstrate protective qualities in animal models, as demonstrated in the following examples, these identified sequences, when expressed as antigens, will be efficacious as a vaccine in animals and particularly in humans. Administration of at least one of the identified antigens is effective to induce an immune response in animals, particularly humans. In one embodiment, the antigen comprises the amino acid sequence set forth as SEQ ID NO:4, SEQ ID NO:6, SEQ ID NO:10, SEQ ID NO:12, SEQ ID NO:16, SEQ ID NO:20 or SEQ ID NO:22. In other embodiments, the antigen comprises the amino acid sequence set forth as SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22. In some embodiments at least two different antigens are administered to an animal, in an amount effective to induce an immune response. In some of these embodiments, the two different antigens are antigens encoded by SEQ ID NO:4 and SEQ ID NO:6. In other embodiments at least three different antigens are administered to an animal, in an amount effective to induce an immune response. In some of these embodiments, two of the different antigens are antigens encoded by SEQ ID NO: 4 and SEQ ID NO: 6, and a third different antigen is selected from the group: SEQ ID NO:8, SEQ ID NO: 10, SEQ ID NO:12, SEQ ID NO:14, SEQ ID NO:16, SEQ ID NO:18, SEQ ID NO:20 or SEQ ID NO:22. In other embodiments, the two different antigens are selected from the group: SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20 or SEQ ID NO:22, while the third different antigen is selected from the group: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38.

### EXAMPLES

The following examples are included to demonstrate preferred embodiments of the application. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

MATERIALS AND METHODS. Chlamydia pneumoniae. Chlamydia pneumoniae strain CDC/CWL-029 (ATCC VR-1310) was grown, purified and quantified as described by Vaglenov et al 2005. Briefly, Buffalo Green Monkey Kidney cells (Diagnostic Hybrids, Inc. Athens, Ohio) were used as host cells for propagation of chlamydiae. For purification, embroid bodies in supernatant culture medium were concentrated by sedimentation, followed by low-speed centrifugation for removal of host cell nuclei, and by step-gradient centrifugation of the supernatant in a 30% RenoCal-76-50% sucrose step-gradient. Sediments of purified infectious EBs were suspended in sucrose-phosphate-glutamate (SPG) buffer and stored at −80° C.

Animals. Inbred A/J and C57BL/6 female mice were obtained from Harlan Sprague Dawley, Inc. (Indianapolis, Ind.) at 5 weeks of age. Udel “shoebox” type cages with spun fiber filter top were maintained in static air or ventilated cage racks. Five animals were housed per cage in a temperature-controlled room with a 12-hour light/dark cycle, with ad libitum access to water and one of two diets. Mice were fed a 19% protein/1.33% L-arginine standard rodent maintenance diet. Beginning two weeks before challenge infection and during challenge infection, mice were fed a custom 24% protein/1.8% L-arginine diet (Harlan Teklad, Madison, Wis.). All components except protein/L-arginine were similar to the standard rodent maintenance diet. The custom diet was used because it was associated in preliminary experiments with enhanced immune responses and lower variance than the standard diet composed of non-chemically defined nutrient components. All animal protocols followed NIH guidelines and were approved by the Auburn University Institutional Animal Care and Use Committee (IACUC).

Negative and positive controls. In all experiments, unvaccinated (naïve) but challenged animals served as negative protection controls, and mice immunized with 5×10⁶ genomes of viable Chlamydia pneumoniae one month prior to the vaccine challenge served as positive protection controls (immune). Groups were scored for protection by calculating the percent lung weight increase over that of age-matched unchallenged female A/J mice (138.4 mg), and by calculating the mean logarithm of total Chlamydia pneumoniae per lung. These values were then converted to a relative protection score by normalizing them to the lung weight increase or logarithm of total lung Chlamydia pneumoniae load that was calibrated by control immune (protection score 1=100% protection) and naïve (protection score 0=0% protection) groups. A CMVi-UB LEE construct encoding the luciferase gene (LUC) served as a control for LEE-based immunizations, and a plasmid construct pCMVi-UB carrying the same LUC insert was used as the control for plasmid-based immunizations.

Chlamydia pneumoniae lung challenge infection. Mouse intranasal inoculation was performed as described by Huang et al (1999), and optimal doses for live-immunization and challenge inocula were determined in preliminary experiments. For intranasal inoculation, mice received a light isoflurane inhalation anesthesia. Vaccine protection control mice were inoculated with a low dose of 5×10⁶ Chlamydia pneumoniae elementary bodies in 30 μl SPG buffer. In rounds 1 and 2, higher-dose challenge infection was performed 4 weeks after the last gene gun genetic vaccination or low dose inoculation of live Chlamydia pneumoniae, by intranasal inoculation of 1×10⁸ Chlamydia pneumoniae elementary bodies in 30 μl SPG buffer. In round 3, mice were challenged by an LD₅₀ dose of 5×10⁸ Chlamydia pneumoniae elementary bodies in 30 μl SPG buffer. Mice were sacrificed by CO₂ inhalation 2 hours, 3 days, 10 days, or 15 days after inoculation, and lungs and spleen were weighed, snap frozen in liquid nitrogen, and stored at −80° C. until further processing. In all screening experiments, mice were sacrificed 10 days after inoculation. From selected animals, terminal blood was collected in heparinized microcentrifuge tubes by axillary incision under isoflurane anesthesia. Plasma was obtained by centrifugation at 5,000×g for 20 min in a microcentrifuge. Percent lung weight increase was based on naïve lung weights of 138.4 mg for adult A/J mice and 133 mg for adult C57BL/6 mice.

Mouse lung nucleic acid extraction. Mouse lungs were homogenized in guanidinium isothiocyanate Triton X-100-based RNA/DNA stabilization reagent in disposable tissue grinders (Fisher Scientific, Atlanta, Ga.) to create a 10% (wt/vol) tissue suspension. This suspension was used for total nucleic acid extraction by the High Pure® PCR template preparation kit (Roche Applied Science, Indianapolis, Ind.) and for mRNA extraction using oligo (dT)₂₀ silica beads.

For mRNA extraction, a suspension of oligo (dT)₂₀-coated silica beads (25 mg/ml in dH₂O; 1 μm particle size, Kisker GbR, Steinfurt, Germany) was used. First, 100 μl of 10% lung suspension was mixed with 10 μl oligo (dT)₂₀ silica bead suspension diluted in 230 μl dilution buffer (0.1 M Tris-HCl, pH 7.5, 0.2 M LiCl, 20 mM EDTA). For mRNA binding, samples were incubated at 72° C. for 3 minutes followed by room temperature for 10 minutes. The silica beads were sedimented by centrifugation at 13,000×g for 2 minutes, supernatants removed by decanting, the beads resuspended in 100 μl DNase buffer (20 mM Tris-HCl, pH 7.0, 1 M NaCl, 10 mM MnCl₂) containing 100 U of RNase-free bovine pancreatic DNase I (Roche Applied Science, Indianapolis, Ind.) and incubated for 15 minutes at room temperature. Subsequently, beads were washed three times with wash buffer (10 mM Tris-HCl, pH 7.5, 0.2 M LiCl, 1 mM EDTA) by vigorous vortexing for 2 minutes followed by sedimentation at 13,000×g, and mRNA was eluted by resuspension of the beads in 200 μl DEPC-treated ddH₂O followed by incubation at 72° C. for 7 minutes, centrifugal sedimentation, and removal of the supernatant mRNA. The purified nucleic acids samples were stored at −80° C. until used for real-time PCR assays.

Analysis of lung nucleic acids by real-time PCR. The primers and probes used in all PCR assays were custom synthesized by Operon, Alameda, Calif. The copy number of Chlamydia pneumoniae genomes per lung was determined by Chlamydia genus-specific 23S rRNA FRET (fluorescence resonance energy transfer) qPCR. One-step duplex RT-qPCR for analysis of lung transcript concentrations was performed in a Lightcycler as described by Wang et al (2004). RT reaction and PCR amplification for the analyte transcripts and an internal reference housekeeping gene transcript (porphobilinogen deaminase, PBGD) were performed in the same tube. All analyte transcript concentrations are expressed as copies per 1000 PBGD reference transcripts. Tim 3 is a CD4 Th1 cell-specific surface protein (GenBank #AF450241), GATA-3 is a CD4 Th2 cell-specific GATA sequence transcription factor (GenBank #X55123), CD45RO is a memory T cell surface protein (GenBank #NM_{—}0112100).

Data analysis. All analyses were performed with the Statistica 7.0 software package (StatSoft, Tulsa, Okla.). Data of Chlamydia pneumoniae genome copies, RT-PCR gene transcripts, and anti-Chlamydophila IgG1 and IgG2a antibody relative light unit values were logarithmically transformed. Normal distribution of data was confirmed by the Shapiro-Wilk's W test, and homogeneity of variances by Levene's test. Data were evaluated by mean plots ±95% confidence intervals, and analyzed by analysis of variance (ANOVA). Post-hoc comparisons of means were performed under the assumption of no a priori hypothesis by the Tukey honest significant difference (HSD) test, or by Dunnett's test for determination of the significant differences between a single control group mean and the remaining treatment group means. Survival data were analyzed by one-sided Fisher's Exact test.

Experimental Outline: Round 1—ELI screen of the complete Chlamydia pneumoniae genome. The genome sequence of Chlamydia pneumoniae isolate CDC/CWL-029 (ATCC strain VR-1310) was extracted from Genbank (AE001363, 1,230,230 bp). The 1,052 annotated genes of Chlamydia pneumoniae were imported into a gene-splitting and primer prediction program; primer pairs to amplify 1,263 open reading frames (ORFs) of 1.5 kb or less were exported. A 1.5 kb maximum ORF length was chosen to ensure sufficient polymerase chain reaction (PCR) quality and yields, and this generated additional fragments. The sequence-specific primers each carried at the 5′-end a common 15 base stretch in which deoxy-uracil bases were interspersed every third position. This design rendered the 5′-ends of all PCR products susceptible to uracil-DNA-glycosylase (UDG) cleavage. Genomic DNA was isolated from purified Chlamydia pneumoniae stock as described by Sykes et al (1996), and used as a template. All products were PCR-amplified from Chlamydia pneumoniae genomic DNA.

First-pass PCR conditions were: 20 cycles of 94° C. for 1 minute, 55° C. for 1 minute, 72° C. for 2 minutes followed by 25 cycles of 94° C. for 1 minute, 50° C. for 1 minute, 72° C. for 2 minutes and lastly 72° C. for 7 minutes. This amplified all but 364 ORFs. Failed PCR reactions were repeated at different annealing temperatures, and all but 38 were amplified. New primers were synthesized, but not re-designed and all but 16 ORFs were amplified. These ORFs were amplified with new, re-designed primers. ORF PCR products were purified by gel-filtration with Sephadex G-50. The purified PCR products were vacuum-concentrated in a Speedvac centrifuge as needed to keep the volume below 200 μl. The pooled PCR products were phenol: chloroform extracted, chloroform extracted and ethanol precipitated.

The products were arrayed into microtiter wells for pooling. The ORFs were combined into 90 pools of approximately 42 ORFs. Each ORF was a member of three unique pools, and the complete genomic set of ORFs is represented in three different sets of 30 pools. This pooling strategy can be conceptualized as a 3-dimensional grid. The purpose is to enable multiplex analyses of the subsequent ELI results and thereby facilitate the selection power of the screen.

To enable non-covalent linkage of expression elements, the ORF pools were exposed to UDG. These samples were combined with 3 expression elements also produced by PCR: the CMV promoter linked to a ubiquitin sequence, the CMV promoter linked to a secretory leader sequence, and a terminator sequence. These were also designed with UDG sensitive ends and prepared for ORF linkage by enzymatically exposing 3′ single stranded ends complementary to the ORFs.

A/J mice were used in all vaccine screening experiments. For Helios gene gun immunization (Bio-Rad Laboratories, Hercules, Calif.), mice received an isoflurane inhalation anesthesia, and were immunized on the outside of each ear. Three gene gun immunizations were performed in one month intervals with 5 mice per vaccine pool. The individual vaccine dose per ORF LEE was approximately 50 ng DNA/mouse (1/42 dose), resulting in a total DNA dose of approximately 2 μg DNA/mouse per pool, split into two immunization doses per mouse.

The numbers of Chlamydia pneumoniae genomes per lung were logarithmically transformed, and the means of all immunization pools determined. The protective capacity of each pool of ˜42 ORF immunization constructs was determined as protection score in a linear equation in which the Log₁₀ of the lung Chlamydia pneumoniae genomes of the low-dose immunized positive protection controls equaled 100% protection, and that of the naïve controls 0% protection. Groups that had higher Chlamydia pneumoniae lung loads than the naïve controls had negative protection scores. The protective potential of the Chlamydia pneumoniae ORFs was matrix analyzed in two ways: 1) by ranking in descending order the sum of the protection scores of the X, Y, and Z pools in which any one ORF was a member, and 2) by residency of an ORF in 3 protective groups (1 each from the x, y, and z sets), which represents an intersection of planes X, Y, and Z. Using both analyses of inferred protection, 46 candidates were identified.

Round 2—Initial Chlamydia pneumoniae vaccine candidate screen. After the total Chlamydia pneumoniae genome screen, the 46 highest scoring inferred candidates were tested individually. Subsequent steps were identical to those described above for Round 1. The inocula per gene gun-dose were comprised of 200 ng of the candidate ORF and 800 ng of pUC118 filler DNA. Each mouse received 2 doses and each group had 10 mice. All other gene gun vaccination parameters were identical to the round 1 experiment.

Round 3—Confirmatory Chlamydia pneumoniae vaccine candidate screen. After the round 2 screen of 46 candidates, the highest ranked 12 candidates were cloned as full genes, excluding ide and Cpn0095 for which only the identified fragments ide_b, ide_ab, and Cpn0095_a were tested. The candidates were tested individually in a high-dose Chlamydia pneumoniae challenge using an inoculum that in titration experiments killed 50% of inoculated naïve mice within 10 days. This experiment was designed as a rigorous challenge of the protective efficacy of the final candidate genes, with a multiple readout evaluating protection from disease by survival of mice and determination of lung weight increase, as well as elimination of Chlamydia pneumoniae organisms by determination of total chlamydial lung loads.

Genetic immunization was again performed by ballistic delivery of recombinant mammalian expression vectors carrying individual bacterial genes under control of a eukaryotic promoter. This genetic immunization vector, pCMVi-UB is described in FIG. 2. Bacterial sequences were PCR amplified from Chlamydia pneumoniae genomic DNA with sets of gene-specific primers using to following two phase protocol. For Phase 1, 2.0 μl 5xiProof buffer (BioRad), 0.2 ul 10 mM dNTP (Promega), 1.0 μl 1 uM forward gene-specific primer, 1.0 μl 1 μM reverse gene-specific primer, 1.0 μl genomic DNA (0.4 ng/ul), 0.1 μl iProof DNA pol (5 unit/μl), and 4.7 μl water were mixed and thermally cycled as follows: 98° C., 30 sec, followed by 5 times 98° C., 10 sec, 50° C., 30 sec, and 72° C., 15 sec, 20 times 98° C., 10 sec, 62° C., 30 sec, 72° C., 15 sec/kb, followed by 72° C., 7 min. Phase 2 used the entire 10 μl volume of the phase 1 reaction, combined with 10 μl 10×Taq DNA pol buffer (Promega), 2 μl 10 mM dNTP (Promega), 2.5 μl 10 μM universal forward dU primer, 2.5 μl 10 μM universal reverse dU primer, 1 μl Taq DNA pol (1 unit/μl), and 72 μl water. The thermal cycling conditions were 95° C., 2 min, followed by 5 times 94° C., 30 sec, 50° C., 30 sec, 72° C., 1.5 min, 15 times 94° C., 30 sec, 64° C., 30 sec, 72° C., 1.5 min/kb, followed by 72° C., 10 min.

The PCR generated fragments were dU cloned into the specially prepared pCMVi-UB vector. The vector was cleaved at BglII and HindIII sites and synthetic single stranded adapters were ligated to the imbedded 3′ ends of the cleavage sites. This resulted in generation of protruded 3′ ends. Adapter sequences were designed to compliment the ends of the PCR products added during the second phase of the protocol. To generate 3′ protruded ends on the PCR products they were treated with UGPase. This removed the primer incorporated dU bases from the 5′ ends of the PCR products and exposed complementary to the adaptors 3′ ends. The prepared vector and UDGase treated PCR product were mixed together and without any additional steps used for bacterial transformation. Correct integration and sequence of the assembled expression cassettes was confirmed by sequencing.

Plasmid-coated gold particles for gene gun immunization were prepared in a standard protocol (BioRad, Inc.) using endotoxin free plasmid DNA preparations. Each vaccine dose contained total of 1 μg of a plasmid DNA mix. The mix contained 0.9 μg of an antigen encoding plasmid and 0.1 μg of a genetic adjuvant. This adjuvant was a 1:4 mixture of two plasmids encoding the B and A subunits of E. coli heat-labile toxin. The coding sequence for subunit A was modified to change the R at position 192 to G to detoxify the gene. DNA was delivered by gene gun (BioRad, Inc.) into each ear lobe of each mouse (10 mice/group). An accelerated vaccination schedule was used to immunize mice on days 0, 3, 6, 20, and 34. Mice were challenged with 5×10⁸ Chlamydia pneumoniae elementary bodies 4 weeks after the last immunization.

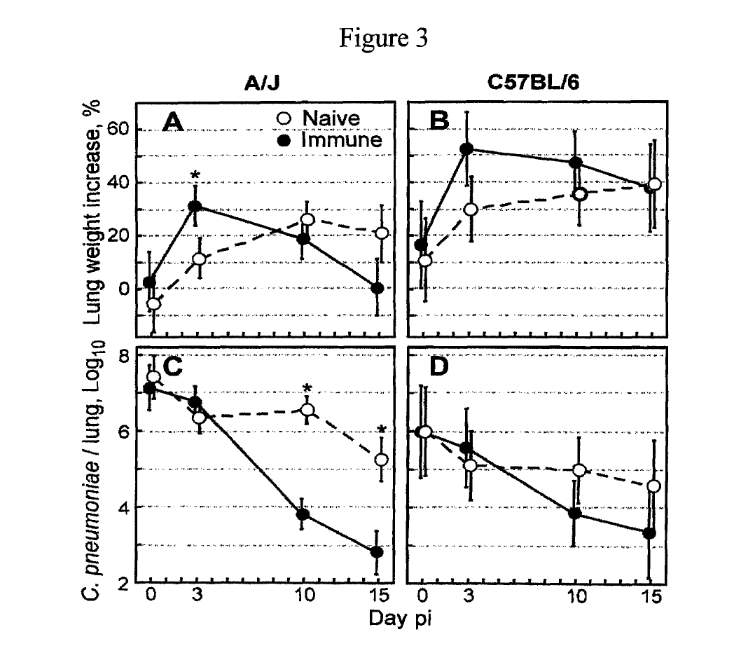

RESULTS. Referring now to FIG. 3, prior to conducting the vaccine screen, several parameters of the murine model deemed important for an optimal challenge-protection assay were evaluated. Specifically, two mouse strains, A/J and C56BL/6, were evaluated. These strains were chosen because of their known differences in inflammatory responses and putatively divergent susceptibility to Chlamydia pneumoniae disease. To calibrate the range of achievable protection and to provide control, groups of naïve and immunized mice were prepared by intranasal inoculation with 5×10⁶ live Chlamydia pneumoniae EBs or by mock inoculation four weeks before the high dose challenge with 10⁸ organisms. Total lung load of Chlamydia pneumoniae and lung weight increase were used as readouts for protection.

A fifteen day time course of infection was analyzed in both mouse strains, each strain having naïve and immune to Chlamydia pneumoniae mice. A/J mice had a lower incidence of disease than C57BL/6 mice, expressed as percent increase over the average lung weight of unchallenged mice. As shown in FIGS. 3A and 3B, disease in immune mice peaked on day 3 post inoculation (pi), and in naïve mice between days 10 and 15 pi. FIGS. 3C and 3D demonstrate that Chlamydia pneumoniae lung loads in naïve mice, determined by real-time PCR as genome copies per lung, tended to be lower in C57BL/6 mice than in A/J mice, but significantly lower only on day 10 (p=0.038). On days 0 and 3 pi, lung loads of immune mice were not different from naïve mice. On days 10 and 15 pi, lung loads of Chlamydia pneumoniae in immune A/J mice were approximately 300-fold reduced (2.5 log reduction) as compared to naïve A/J mice (p<0.001), while lung loads of immune and naïve C57BL/6 mice did not differ significantly. The strong elimination of Chlamydia pneumoniae by immune A/J mice identifies A/J mice, but not C57BL/6 mice, as suitable for identification of Chlamydia pneumoniae vaccine candidates. The kinetics of elimination of Chlamydia pneumoniae in A/J mice indicate that day 10 post inoculation is the optimum time point for identification of vaccine candidates that promote immune elimination of Chlamydia pneumoniae organisms. At later time points, the immune response induced by the challenge inoculation potentially interferes with pre-existing immunity against vaccine candidates, preventing the identification of protective Chlamydia pneumoniae antigens.

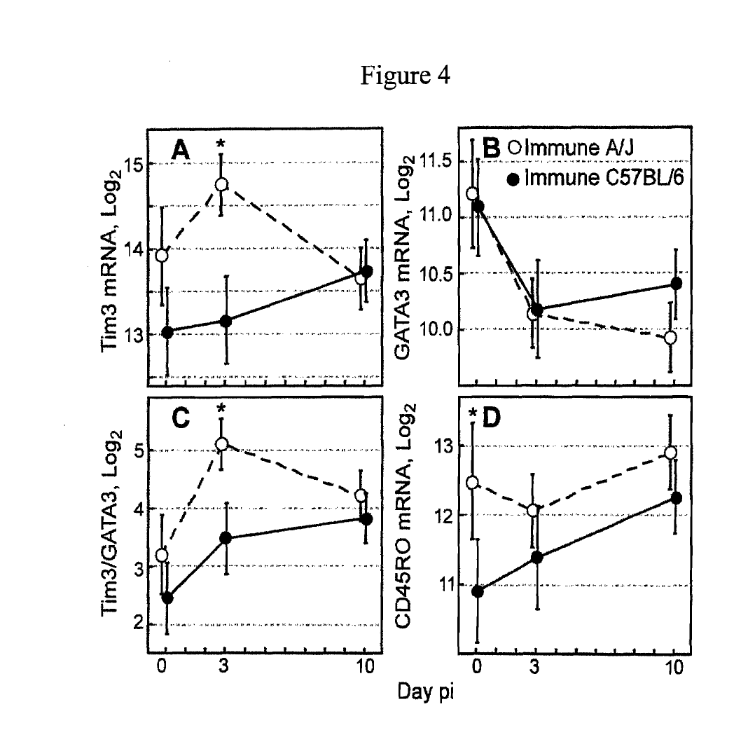

Turning now to FIG. 4, also prior to the vaccine screen, the levels of several key immune-related transcripts were evaluated as indicators of the type and intensity of the local lung tissue response to the Chlamydia pneumoniae challenge. Early Tim3 transcripts, which are indicative of Th1 cells, peaked on day 3 pi in A/J mice and were significantly higher than in C57BL/6 mice (p=0.004; FIG. 4A). Early GATA3 transcripts, which are indicative of Th2 cells, did not differ between the mouse strains (FIG. 4B). Thus, the ratio of Tim3/GATA3 was significantly higher in A/J mice than in C57BL/6 mice (p<0.001; FIG. 4C), consistent with a Th1-biased immune profile for A/J mice. CD45RO transcripts, indicating memory T cells, were higher in A/J mice (p=0.008 for combined day 0, 3, and 10 data; FIG. 4D). This data demonstrates that pre-immunized A/J mice mount a stronger and more Th1 biased early immune response than C57BL/6 mice during challenge with Chlamydia pneumoniae. The data further confirms that those A/J mice are appropriate for a respiratory challenge model for identification of Chlamydia pneumoniae vaccine candidates.

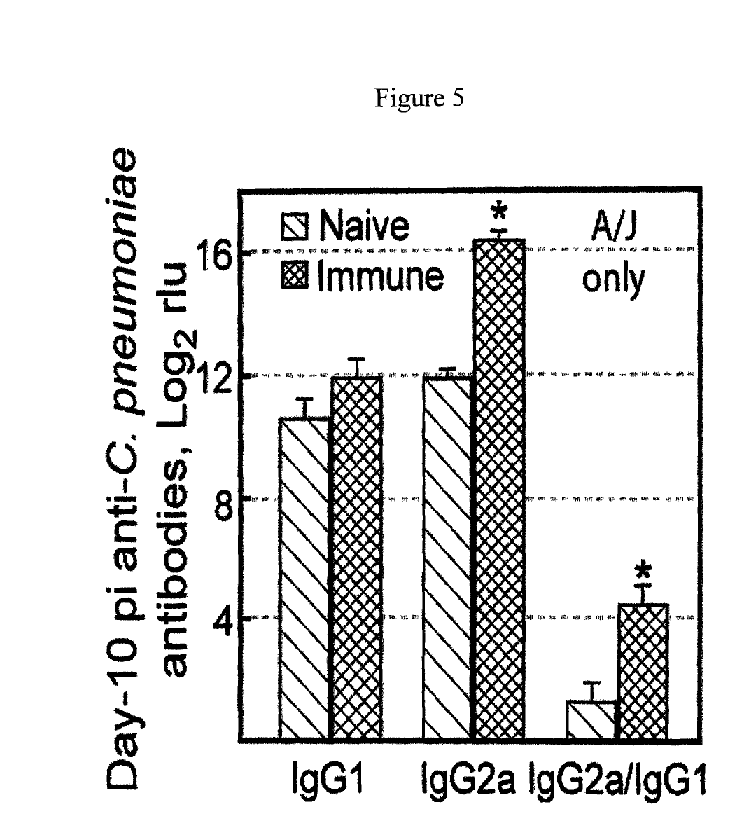

FIG. 5 demonstrates day-10 pi plasma antibody responses against Chlamydia pneumoniae of naïve and immune A/J mice as determined by ELISA. Absolute levels and the ratio of IgG2a (Th1-associated) and IgG1 (Th2-associated) antibodies confirmed a highly significant Th1 shift of the immune response to Chlamydia pneumoniae in immune as compared to naïve A/J mice (p<0.001).

Accordingly, conditions were identified that maximized the amplitude between chlamydial lung burden of naïve mice and of immune mice protected by a low-dose live Chlamydia pneumoniae inoculation. These preliminary results demonstrate that the optimum protection readout time point is ten days after challenge infection, and that A/J, but not C57BL/6 mice, are the host inbred mouse strain in the respiratory challenge model suitable for identification of protective Chlamydia pneumoniae protein antigens. The corollary of this finding is that an appropriate host genetic background will be essential for protective efficacy of any Chlamydia pneumoniae vaccine, presumably not only in inbred mouse strains, but also in an outbred human vaccine population. Vaccine candidates identified using this animal model of disease will be chlamydial antigens that are presented to and recognized by the immune system in a manner that stimulates a productive host response. However, successful use of vaccine antigens in individuals that are genetically refractory to immune protection against Chlamydia pneumoniae, as are C57BL/6 mice, will require an understanding of the factors that normally prevent immune protection. This will enable immunity to be manipulated more productively.

Referring now to FIG. 5, the Round 1 genomic screen for vaccine candidates identified protective open reading frames that were common sonstitutuents of a positively scored X, Y, and Z ELI pool. This represents the virtual equivalent of the intersections of all positively scored cubic planes. Each individual ORF was also assigned a genomic score by summing the relative protection scores corresponding to its 3 resident pools. The ranking of the protections scores was used as the primary criterion and intersections of positively scored cubic planes as secondary criterion, to select 46 Chlamydia pneumoniae ORFs for individual vaccine candidate screening as set forth in Table 3 below. Table 3 demonstrates the genetic vaccine fragments of Chlamydia pneumoniae genes selected in Round 1 for further testing in Round 2, and selected in Round 2 for final testing in Round 3.

TABLE 3

| | | Round-1 Total Lung | | Round-2 Total Lung | |
|---|---|---|---|---|---|
| | Vaccine | pneumoniae | Round-1 | C. pneumoniae | Round-2 |
| Gene | fragments | Protection Score | Rank | Protection Score^{a} | Rank^{b} |
| mutL_a | 2 | 0.729 | 1 | 0.102 | 21 |
| Idh | 1 | 0.680 | 2 | −0.074 | 36 |
| atoC | 1 | 0.663 | 3 | 0.349 | 11 |
| CPn0249_b | 2 | 0.651 | 4 | −0.295 | 45 |
| gapA | 1 | 0.622 | 5 | −0.273 | 44 |
| ide_b | 3 | 0.621 | 6 | 0.857 | 3 |
| CPn0884 | 1 | 0.614 | 7 | 0.042 | 30 |
| CPn0913 | 1 | 0.554 | 8 | 0.039 | 31 |
| fabD | 1 | 0.544 | 9 | 0.484 | 9 |
| cutE_a | 2 | 0.542 | 10 | 1.287 | 1 |
| CPn0420 | 1 | 0.541 | 11 | 1.102 | 2 |
| CPn0755 | 1 | 0.539 | 12 | 0.071 | 24 |
| ppa | 1 | 0.537 | 13 | 0.230 | 15 |
| yigN | 1 | 0.521 | 14 | −0.163 | 40 |
| efp_2 | 1 | 0.519 | 15 | −0.098 | 38 |
| glgX_b | 2 | 0.514 | 16 | 0.559 | 6 |
| CPn0330 | 1 | 0.512 | 17 | 0.125 | 18 |
| CPn0095_a | 2 | 0.508 | 18 | 0.276 | 13 |
| CPn0020_b | 2 | 0.502 | 19 | 0.524 | 7 |
| CPn0174 | 1 | 0.502 | 20 | −0.017 | 34 |
| ychM_a | 2 | 0.496 | 21 | −0.109 | 39 |
| CPn1072 | 1 | 0.495 | 22 | 0.086 | 22 |
| CPn0044 | 1 | 0.495 | 23 | 0.133 | 17 |
| CPn0155 | 1 | 0.495 | 24 | 0.228 | 16 |
| CPn0523 | 1 | 0.492 | 25 | −0.214 | 42 |
| oppA_2_a | 2 | 0.489 | 26 | 0.762 | 4 |
| CPn0554 | 1 | 0.488 | 27 | 0.043 | 28 |
| yacE | 1 | 0.484 | 28 | −0.298 | 46 |
| CPn0830 | 1 | 0.479 | 29 | −0.086 | 37 |
| flil | 1 | 0.476 | 30 | 0.118 | 19 |
| rl1 | 1 | 0.472 | 31 | 0.401 | 10 |
| CPn0509 | 1 | 0.460 | 33 | 0.524 | 8 |
| CPn0981_b | 2 | 0.458 | 34 | 0.025 | 33 |
| CPn1020_b | 2 | 0.438 | 37 | −0.197 | 41 |
| pyk | 1 | 0.433 | 40 | 0.047 | 27 |
| ftsH_a | 2 | 0.416 | 43 | 0.053 | 26 |
| CPn1061 | 1 | 0.414 | 44 | 0.070 | 25 |
| CPn0927 | 1 | 0.405 | 47 | 0.316 | 12 |
| CPn1070 | 1 | 0.405 | 48 | 0.028 | 32 |
| gidA_b | 2 | 0.403 | 49 | 0.112 | 20 |
| CPn0553 | 1 | 0.396 | 50 | 0.076 | 23 |
| rs5 | 1 | 0.392 | 53 | 0.043 | 29 |
| CPn0602 | 1 | 0.345 | 70 | −0.218 | 43 |
| ssb | 1 | 0.308 | 94 | 0.582 | 5 |
| CPn0369 | 1 | 0.299 | 100 | −0.038 | 35 |
| pbp2_b | 3 | 0.290 | 109 | 0.244 | 14 |

^{a}Bold numbers indicate significant difference (p < 0.05) from naïve controls in a post-hoc Dunnett's test for determination of the significant differences between a single control group mean and the remaining treatment group means in ANOVA.
^{b}Bold numbers indicate genes selected for further testing in round 3.

The 46 individual Chlamydia pneumoniae partial or full-length ORFs selected in round 1 were subsequently screened as individual LEEs in Round 2 as described above. Total lung Chlamydia pneumoniae protection scores, and the ranking of the genes based on these scores is shown in the last 2 columns of Table 3 above. The results of Round 2 selected the following Chlamydia pneumoniae genes, in this ranking, as candidates for final testing and confirmation in Round 3: cutE (SEQ ID NOS:1-4), Cpn0420 (SEQ ID NOS:5-6), ide (SEQ ID NOS:7-12), oppA_{—}2 (SEQ ID NOS:17-20), ssb (SEQ ID NOS: 21-22), glgX (SEQ ID NOS:27-30), Cpn0020 (SEQ ID NOS:31-34), Cpn0509 (SEQ ID NOS:23-24), fabD (SEQ ID NOS:25-26), rl1 (SEQ ID NOS:37-38), atoC (SEQ ID NOS:35-36), and Cpn0095 (SEQ ID NOS:13-16).

In Round 3, the identified final 12 candidates were cloned as full-length genes into genetic immunization plasmid CMVi-UB (FIG. 1), except for ide and Cpn0095, which were cloned as fragments ide_ab and Cpn0095_a. Mice were genetically vaccinated with these constructs together with a genetic vaccine adjuvant composed of plasmids expressing mutant, non-toxic E. coli enterotoxin A and B subunits. A 5-fold increased challenge inoculum of 5×10⁸ Chlamydia pneumoniae elementary bodies was used that elicited severe disease and was lethal for approximately 50% of intranasally inoculated naïve female A/J mice (LD₅₀). The high-dose challenge was used to evaluate to total protective efficacy of the vaccine candidates for prevention of Chlamydia pneumoniae-induced death and lung disease, as well as the efficacy in eliminating the agent.

The survival data is detailed in Table 4 below, and indicates that along with the calibration live vaccine, genes cutE, Cpn0420, and Cpn0020 prevented death of any inoculated animal while 43% of naïve mice died (P<0.05, Fisher Exact test). Table 4 demonstrates the survival of high-Chlamydia pneumoniae dose-challenged mice in Round 3 vaccinated with plasmid-cloned Chlamydia pneumoniae genes selected in Round 2 for further testing. Bold numbers indicate significant difference (p<0.05) from naïve controls in Fisher Exact test. In all groups vaccinated with the remaining constructs, one or more animals died, and the survival in these groups was not significantly different from naïve mice. Thus, genes cutE, Cpn0420, and Cpn0020 mediated significant protection from Chlamydia pneumoniae-induced death.

TABLE 4

| Vaccine | 10-day survival | %10-day survival^{a} |
|---|---|---|
| Naïve | 8/14 | 57 |
| Live vaccine | 15/15 | 100 |
| Control vaccine | 9/10 | 90 |
| cutE | 10/10 | 100 |
| Cpn0420 | 10/10 | 100 |
| ide_ab | 9/10 | 90 |
| oppA_2 | 9/10 | 90 |
| ssb | 9/10 | 90 |
| Cpn0509 | 9/10 | 90 |
| fabD | 3/10 | 30 |
| glgX | 7/10 | 70 |
| Cpn0020 | 10/10 | 100 |
| atoC | 8/10 | 80 |
| rl1 | 8/10 | 80 |

^{a}Bold numbers in red indicate significant difference (p < 0.05) from Naïve controls in Fisher Exact test.

Turning now to FIG. 7 and Table 5, below, the efficacy of the vaccine constructs in reducing Chlamydia pneumoniae-induced lung disease (interstitial bronchopneumonia) was evaluated by analyzing lung weight increases of surviving challenged mice when they were sacrificed on day 10 after inoculation. It is well known in the art that lung weight increase over unchallenged matched animals is proportional to lung infiltration with inflammatory cells, and therefore reflects disease intensity. Table 5 demonstrates Round-3 protection scores based on the day-10 lung weight increase (over unchallenged mice; equals protection from disease) of high-Chlamydia pneumoniae dose-challenged mice in Round 3 vaccinated with plasmid-cloned Chlamydia pneumoniae genes. Table 5 and FIG. 7 indicate that genes cutE, Cpn0420, oppA_{—}2, and ssb mediate significant protection from lung disease (p <0.05, Dunnett's test).

TABLE 5

| | Round-3 Lung Weight Increase | P for difference to |
|---|---|---|
| Vaccine^{a} | Protection Score^{b} | Naïve controls^{c} |
| Naïve | 0.000 | |
| Live vaccine | 1.000 | 0.002 |
| Control vaccine | 0.330 | 0.543 |
| cutE | 0.827 | 0.029 |
| Cpn0420 | 0.948 | 0.009 |
| ide_ab | 0.648 | 0.126 |
| oppA_2 | 1.139 | 0.002 |
| ssb | 1.033 | 0.005 |
| Cpn0509 | 0.761 | 0.058 |
| fabD | 0.404 | 0.605 |
| glgX | 0.516 | 0.304 |
| Cpn0020 | 0.530 | 0.230 |
| atoC | 0.558 | 0.231 |
| rl1 | 0.350 | 0.526 |

^{a}Naïve n = 8, live vaccine n = 15; genetic vaccine groups n = 3-10.
^{b}Dead mice were treated as missing data.
^{c}Bold numbers indicate significant difference (p < 0.05) from Naïve controls in Dunnett's post-hoc test.

Finally, referring to FIG. 8 and Table 6 below, efficacy of the final vaccine candidates in enhancing elimination of Chlamydia pneumoniae as compared to naïve mice was evaluated. To maximize sample size, protection scores based on the logarithm of total Chlamydia pneumoniae lung loads on day 10 from rounds 2 and 3 were combined. Protection scores relate the efficacy individual vaccines to the calibration naïve and live-vaccine groups and therefore normalize between experiments. This also combined efficacy of round-2 LEE-based vaccination with gene fragments (cutE_a, ide_b, Cpn0095_a, oppA_{—}2_a, glgX_b, Cpn00200_b) or full-length genes (Cpn0420, ssb, Cpn0509, fabD, atoC, rl1) with the plasmid-based vaccination with gene fragments (ide_ab, Cpn0095_a) or full-length genes (cutE, Cpn0420, oppA_{—}2, ssb, Cpn0509, fabD, glgX_b, Cpn0020, atoC, rl1). Cpn0095_a had been used in separate Round-2 experiments both as LEE and as plasmid. Table 6 and FIG. 8 demonstrate that genes cutE, Cpn0420, ide, Cpn0095, and oppA_{—}2 mediated significantly enhanced elimination of Chlamydia pneumoniae (p<0.05, Dunnett's test).

TABLE 6

| | Round-3 Total Lung | |
|---|---|---|
| | C. pneumoniae | P for difference to |
| Vaccines^{a} | Protection Score^{b} | Naïve controls^{c} |
| Naïve | 0.000 | |
| Live vaccine | 1.000 | <0.001 |
| Control vaccine | −0.096 | 0.983 |
| cutE_a & full gene | 0.853 | <0.001 |
| Cpn0420 | 0.703 | 0.006 |
| ide_b & ide_ab | 0.610 | 0.024 |
| Cpn0095_a | 0.607 | 0.011 |
| oppA_2_a & full gene | 0.600 | 0.028 |
| ssb | 0.511 | 0.075 |
| Cpn0509 | 0.450 | 0.135 |
| fabD | 0.401 | 0.302 |
| glgX_b & full gene | 0.385 | 0.258 |
| Cpn0020_b & full gene | 0.339 | 0.301 |
| atoC | 0.248 | 0.526 |
| rl1 | 0.246 | 0.530 |

^{a}Naïve, live vaccine groups n = 60; genetic vaccine groups n = 13-20.
^{b}Dead mice were treated as missing data.
^{c}Bold numbers indicate significant difference (p < 0.05) from Naïve controls in Dunnett's post-hoc test.

In summary, cutE and Cpn0420 are identified as genes individually protective by all criteria (survival, disease reduction, Chlamydia pneumoniae elimination). Gene oppA_{—}2 was protective by dual criteria (disease reduction, Chlamydia pneumoniae elimination), and single criterion-protective genes were ssb (disease reduction), ide and Cpn0095 (Chlamydia pneumoniae elimination), and Cpn0020 (survival).

All of the compositions and methods disclosed and claimed herein can be made and executed without undue experimentation in light of the present disclosure. While the compositions and methods of this application have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

<160> NUMBER OF SEQ ID NOS: 38
<210> SEQ ID NO 1
<211> LENGTH: 1623
<212> TYPE: DNA
<213> ORGANISM: Chlamydia pneumoniae
<400> SEQUENCE: 1
gtgctacgaa tcttttgctt tgttatttct tggtgcctta tagcttttgc tcaaccagat 60
ttaagtggat tcgtttccat attaggagcc gcctgtggtt atggattctt ttggtatagt 120
ctagaaccct taaaaaaacc ctcattacct ctaaggactc tttttgtatc ctgttttttc 180
tggatcttca caatagaggg gattcatttt tcttggatgc tctcggatca atatataggc 240
aaactcatct atttggtatg gcttacatta atcacgattt tgtccgttct attttcagga 300
ttttcttgcc ttctagttgc aatcgtacgt cagaaacgca cagctttttt atggagcctt 360
cctggcgtat gggtcgctat cgagatgctt cgattttatg ggatcttttc tgggatgtcc 420
ttcgattatc ttggttggcc tatgacagcc tctgcttatg gacggcagtt tggcggattt 480
ttggggtggg caggtcagag cttcgctgtc atagctgtaa atatgagctt ttattgtcta 540
ctactgaaaa aacctcatgc taaaatgtta tgggtgctca ctcttctttt gccctatact 600
tttggagcaa ttcattatga gtatcttaaa cacgcgtttc aacaagataa gagagcgctg 660
cgtgtcgctg ttgttcaacc cgcgcatccc cccatacgac cgaaacttaa gtccccaata 720
gtcgtctggg aacaactcct ccaactcgta tccccaatac aacaacccat agatttgctg 780
attttcccag aagtagtcgt gccttttggt aagcataggc aagtctatcc ctatgaatcc 840
tgcgcacatt tattgtcttc ttttgctcca cttcccgaag gtaaggcatt tctatcgaat 900
agtgattgtg ccacagctct gtcacaacac tttcagtgtc cagtaattat tggcttagaa 960
cggtgggtga aaaaagagaa cgttttgtat tggtataact ctgctgaggt aatatcacac 1020
aaaggaattt ccgtaggata cgataagcgt atccttgtgc ctggtggcga atatatacca 1080
ggagggaaat tcggatccct aatttgtaga caactatttc ctaaatatgc tctaggatgc 1140
aagagacttc caggtagacg ttctggagtt gtgcaggtcc gaggtttacc tcgtatcggg 1200
atcaccattt gctacgaaga aactttcggc tatcggttgc aatcctacaa gagacaagga 1260
gccgaactcc ttgttaactt aacaaatgac ggatggtatc ctgaatcacg actccctaaa 1320
gtccatttcc tccatgggat gttgagaaat caagagtttg ggatgccttg cgtgcgagct 1380
tgccaaactg gtgttacagc agctgtggat tctctaggtc gaatactcaa aattcttcct 1440
tatgatacta gagaaactaa agccccctca ggggtattgg aaacctcttt gcctctattt 1500
aattataaaa cgctttatgg gtattgtgga gattacccta tgattttgat agctttctgt 1560
gcagtcagtt atctaggagg aggattctta ggatatcgct tgcttgctaa aaaagaaatt 1620
cga 1623
<210> SEQ ID NO 2
<211> LENGTH: 541
<212> TYPE: PRT
<213> ORGANISM: Chlamydia pneumoniae
<400> SEQUENCE: 2
Val Leu Arg Ile Phe Cys Phe Val Ile Ser Trp Cys Leu Ile Ala Phe
1 5 10 15
Ala Gln Pro Asp Leu Ser Gly Phe Val Ser Ile Leu Gly Ala Ala Cys
20 25 30
Gly Tyr Gly Phe Phe Trp Tyr Ser Leu Glu Pro Leu Lys Lys Pro Ser
35 40 45
Leu Pro Leu Arg Thr Leu Phe Val Ser Cys Phe Phe Trp Ile Phe Thr
50 55 60
Ile Glu Gly Ile His Phe Ser Trp Met Leu Ser Asp Gln Tyr Ile Gly
65 70 75 80
Lys Leu Ile Tyr Leu Val Trp Leu Thr Leu Ile Thr Ile Leu Ser Val
85 90 95
Leu Phe Ser Gly Phe Ser Cys Leu Leu Val Ala Ile Val Arg Gln Lys
100 105 110
Arg Thr Ala Phe Leu Trp Ser Leu Pro Gly Val Trp Val Ala Ile Glu
115 120 125
Met Leu Arg Phe Tyr Gly Ile Phe Ser Gly Met Ser Phe Asp Tyr Leu
130 135 140
Gly Trp Pro Met Thr Ala Ser Ala Tyr Gly Arg Gln Phe Gly Gly Phe
145 150 155 160
Leu Gly Trp Ala Gly Gln Ser Phe Ala Val Ile Ala Val Asn Met Ser
165 170 175
Phe Tyr Cys Leu Leu Leu Lys Lys Pro His Ala Lys Met Leu Trp Val
180 185 190
Leu Thr Leu Leu Leu Pro Tyr Thr Phe Gly Ala Ile His Tyr Glu Tyr
195 200 205
Leu Lys His Ala Phe Gln Gln Asp Lys Arg Ala Leu Arg Val Ala Val
210 215 220
Val Gln Pro Ala His Pro Pro Ile Arg Pro Lys Leu Lys Ser Pro Ile
225 230 235 240
Val Val Trp Glu Gln Leu Leu Gln Leu Val Ser Pro Ile Gln Gln Pro
245 250 255
Ile Asp Leu Leu Ile Phe Pro Glu Val Val Val Pro Phe Gly Lys His
260 265 270
Arg Gln Val Tyr Pro Tyr Glu Ser Cys Ala His Leu Leu Ser Ser Phe
275 280 285
Ala Pro Leu Pro Glu Gly Lys Ala Phe Leu Ser Asn Ser Asp Cys Ala
290 295 300
Thr Ala Leu Ser Gln His Phe Gln Cys Pro Val Ile Ile Gly Leu Glu
305 310 315 320
Arg Trp Val Lys Lys Glu Asn Val Leu Tyr Trp Tyr Asn Ser Ala Glu
325 330 335
Val Ile Ser His Lys Gly Ile Ser Val Gly Tyr Asp Lys Arg Ile Leu
340 345 350
Val Pro Gly Gly Glu Tyr Ile Pro Gly Gly Lys Phe Gly Ser Leu Ile
355 360 365
Cys Arg Gln Leu Phe Pro Lys Tyr Ala Leu Gly Cys Lys Arg Leu Pro
370 375 380
Gly Arg Arg Ser Gly Val Val Gln Val Arg Gly Leu Pro Arg Ile Gly
385 390 395 400
Ile Thr Ile Cys Tyr Glu Glu Thr Phe Gly Tyr Arg Leu Gln Ser Tyr
405 410 415
Lys Arg Gln Gly Ala Glu Leu Leu Val Asn Leu Thr Asn Asp Gly Trp
420 425 430
Tyr Pro Glu Ser Arg Leu Pro Lys Val His Phe Leu His Gly Met Leu
435 440 445
Arg Asn Gln Glu Phe Gly Met Pro Cys Val Arg Ala Cys Gln Thr Gly
450 455 460
Val Thr Ala Ala Val Asp Ser Leu Gly Arg Ile Leu Lys Ile Leu Pro
465 470 475 480
Tyr Asp Thr Arg Glu Thr Lys Ala Pro Ser Gly Val Leu Glu Thr Ser
485 490 495
Leu Pro Leu Phe Asn Tyr Lys Thr Leu Tyr Gly Tyr Cys Gly Asp Tyr
500 505 510
Pro Met Ile Leu Ile Ala Phe Cys Ala Val Ser Tyr Leu Gly Gly Gly
515 520 525
Phe Leu Gly Tyr Arg Leu Leu Ala Lys Lys Glu Ile Arg
530 535 540
<210> SEQ ID NO 3
<211> LENGTH: 915
<212> TYPE: DNA
<213> ORGANISM: Chlamydia pneumoniae
<400> SEQUENCE: 3
gtgctacgaa tcttttgctt tgttatttct tggtgcctta tagcttttgc tcaaccagat 60
ttaagtggat tcgtttccat attaggagcc gcctgtggtt atggattctt ttggtatagt 120
ctagaaccct taaaaaaacc ctcattacct ctaaggactc tttttgtatc ctgttttttc 180
tggatcttca caatagaggg gattcatttt tcttggatgc tctcggatca atatataggc 240
aaactcatct atttggtatg gcttacatta atcacgattt tgtccgttct attttcagga 300
ttttcttgcc ttctagttgc aatcgtacgt cagaaacgca cagctttttt atggagcctt 360
cctggcgtat gggtcgctat cgagatgctt cgattttatg ggatcttttc tgggatgtcc 420
ttcgattatc ttggttggcc tatgacagcc tctgcttatg gacggcagtt tggcggattt 480
ttggggtggg caggtcagag cttcgctgtc atagctgtaa atatgagctt ttattgtcta 540
ctactgaaaa aacctcatgc taaaatgtta tgggtgctca ctcttctttt gccctatact 600
tttggagcaa ttcattatga gtatcttaaa cacgcgtttc aacaagataa gagagcgctg 660
cgtgtcgctg ttgttcaacc cgcgcatccc cccatacgac cgaaacttaa gtccccaata 720
gtcgtctggg aacaactcct ccaactcgta tccccaatac aacaacccat agatttgctg 780
attttcccag aagtagtcgt gccttttggt aagcataggc aagtctatcc ctatgaatcc 840
tgcgcacatt tattgtcttc ttttgctcca cttcccgaag gtaaggcatt tctatcgaat 900
agtgattgtg ccaca 915
<210> SEQ ID NO 4
<211> LENGTH: 306
<212> TYPE: PRT
<213> ORGANISM: Chlamydia pneumoniae
<400> SEQUENCE: 4
Val Leu Arg Ile Phe Cys Phe Val Ile Ser Trp Cys Leu Ile Ala Phe
1 5 10 15
Ala Gln Pro Asp Leu Ser Gly Phe Val Ser Ile Leu Gly Ala Ala Cys
20 25 30
Gly Tyr Gly Phe Phe Trp Tyr Ser Leu Glu Pro Leu Lys Lys Pro Ser
35 40 45
Leu Pro Leu Arg Thr Leu Phe Val Ser Cys Phe Phe Trp Ile Phe Thr
50 55 60
Ile Glu Gly Ile His Phe Ser Trp Met Leu Ser Asp Gln Tyr Ile Gly
65 70 75 80
Lys Leu Ile Tyr Leu Val Trp Leu Thr Leu Ile Thr Ile Leu Ser Val
85 90 95
Leu Phe Ser Gly Phe Ser Cys Leu Leu Val Ala Ile Val Arg Gln Lys
100 105 110
Arg Thr Ala Phe Leu Trp Ser Leu Pro Gly Val Trp Val Ala Ile Glu
115 120 125
Met Leu Arg Phe Tyr Gly Ile Phe Ser Gly Met Ser Phe Asp Tyr Leu
130 135 140
Gly Trp Pro Met Thr Ala Ser Ala Tyr Gly Arg Gln Phe Gly Gly Phe
145 150 155 160
Leu Gly Trp Ala Gly Gln Ser Phe Ala Val Ile Ala Val Asn Met Ser
165 170 175
Phe Tyr Cys Leu Leu Leu Lys Lys Pro His Ala Lys Met Leu Trp Val
180 185 190
Leu Thr Leu Leu Leu Pro Tyr Thr Phe Gly Ala Ile His Tyr Glu Tyr
195 200 205
Leu Lys His Ala Phe Gln Gln Asp Lys Arg Ala Leu Arg Val Ala Val
210 215 220
Val Gln Pro Ala His Pro Pro Ile Arg Pro Lys Leu Lys Ser Pro Ile
225 230 235 240
Val Val Trp Glu Gln Leu Leu Gln Leu Val Ser Pro Ile Gln Gln Pro
245 250 255
Ile Asp Leu Leu Ile Phe Pro Glu Val Val Val Pro Phe Gly Lys His
260 265 270
Arg Gln Val Tyr Pro Tyr Glu Ser Cys Ala His Leu Leu Ser Ser Phe
275 280 285
Ala Pro Leu Pro Glu Gly Lys Ala Phe Leu Ser Asn Ser Asp Cys Ala
290 295 300
Thr Ala
305
<210> SEQ ID NO 5
<211> LENGTH: 288
<212> TYPE: DNA
<213> ORGANISM: Chlamydia pneumoniae
<400> SEQUENCE: 5
atgaacaaaa gtcgtttttt acgtttatgc tgctgtctat gcttttgtgg aagtctcttt 60
tatttctata ttaataagca gaactcgctg acgaaattac gcctcgaaat tccttgttta 120
tctgtacgct tgcgtcagct tgagcagcaa aatatttctt tacgtttttt aattgataaa 180
atagaaagac ctgatcattt gatggaaata gcagctcttc ccgaatacca atatttggaa 240
tatccctcag aagaaagtat cagtctttta tcctatgagc taccgtaa 288
<210> SEQ ID NO 6
<211> LENGTH: 95
<212> TYPE: PRT
<213> ORGANISM: Chlamydia pneumoniae
<400> SEQUENCE: 6
Met Asn Lys Ser Arg Phe Leu Arg Leu Cys Cys Cys Leu Cys Phe Cys
1 5 10 15
Gly Ser Leu Phe Tyr Phe Tyr Ile Asn Lys Gln Asn Ser Leu Thr Lys
20 25 30
Leu Arg Leu Glu Ile Pro Cys Leu Ser Val Arg Leu Arg Gln Leu Glu
35 40 45
Gln Gln Asn Ile Ser Leu Arg Phe Leu Ile Asp Lys Ile Glu Arg Pro
50 55 60
Asp His Leu Met Glu Ile Ala Ala Leu Pro Glu Tyr Gln Tyr Leu Glu
65 70 75 80
Tyr Pro Ser Glu Glu Ser Ile Ser Leu Leu Ser Tyr Glu Leu Pro
85 90 95
<210> SEQ ID NO 7
<211> LENGTH: 2826
<212> TYPE: DNA
<213> ORGANISM: Chlamydia pneumoniae
<400> SEQUENCE: 7
atgttttgga aacttttatg tcctatttta atttgcactt ccctatccat aacatcgtgt 60
gaacagcagt tcaaagtcgt ccccaatcag tgccctttac aagtttccac tcctgccgct 120
gcggaccaga aaattgaaaa gattatttgt agtaacgggc tccctcttct tattatttcc 180
gaccctaatc ttcctacttc gggagcagca ctccttgtga aaacaggaaa taatgccgat 240
cctgaagagt atcctgggat ggcgcacttc acagaacact gtgtctttct tggaaatgaa 300
aagtatcctg aggtctctgg tttccctgga tttttaagcg aaaataatgg ggtgcataat 360
gctttcactt acccaaataa aacagtcttt gtattttcag tagaacattc tgcgttttct 420
gatgctttag accaatttgt tcatctattt attaatccga agtttcgtca agaagatctt 480
gatagagaaa agtacgcagt acatcaagaa ttcgctgctc atcctctttc tgatgggaga 540
cgtgtgcatc gcattcagca gcttgttgct cctcagggcc atccctgcgc acgttttggt 600
tgtgggaatg cttccaccct caccccagtg actacagaga aaatggcaga atggtttaag 660
ctacattatt ctcctgagaa tatgtgtgct attgcttaca catcagctcc gctctctaaa 720
gcaaagaaac agttctcaaa gattttttct cagattccta gatcaaaaaa ttatgaaaga 780
caggaacctt ttcttccttc tggtgacacc tcgtcattaa agaatctcta tattaaccaa 840
gcaattcagc ctacctctaa tctagaaatt tactggcata tttatgaatc ttcccatccg 900
attcctttag gctgttacaa ggctcttgct gaagttttaa gaaatgagag taagaacagt 960
ttagtctctt tattgaaaaa cgagcagcta attacggatt tagacgtgga attctttaga 1020
agttctttaa atactggaga attctatatt agctatgagc ttacggagaa aggcgataaa 1080
cactattctc aggttattga tagtaccttc caatatcttc gatatattca ggaacacggg 1140
attcccaact atacgttaga agaaatttct acaattaatg ctttaaacta ctgttacagt 1200
tccaaaagtc cattgtttga tctgctttgt aagcagattg tatccttggg caatgaggat 1260
ctatctacgt atccttatca tagccttgtg tatcctaaat actcttctga agacgagtct 1320
gctcttctta atttagtctc tgatcctgaa caagcacgtt ttgtcttatc tagtaagaac 1380
tctgagcatt gggaagaagc gactcagctc cacgatccta tttttgacat gacctactat 1440
gtaaaagctc tggacggtgt tcaggattat ggaaaggtgc agtcactaaa gcccatagct 1500
cttccaaagc cgaatctgtt tattcctaaa gaggtgactc ttcctggtgt acacctactg 1560
aaaaaacaag aatttccttt tgctcctgca ctcagttacc aagatgataa attaactctg 1620
taccattgcg aggaccacta ctatacagca ccgaaactct ccagtcagat tcgcatccgc 1680
tctcctcaga tttcgaggtc ctcccctcaa tttctagttg ctacggagct ctattgctta 1740
gctgtgaatg atcagctttt gagggagtat tatcccgcaa cgcaagctgg tctttctttt 1800
acttctgctt taggtggtga tggtattgat ttaagagttt cagggtacac aacaacagtc 1860
cctgcattgt taaactcaat tttaacctca ttacctaatt tagagattag ctatgagaca 1920
ttcttagtat ataaaaagca gttgttagag ctttatcaag gagctttgct caactgtccg 1980
gttcgttctg ggcttgatga gcttgcctca caagttatga aggagacgta ttctaatact 2040
actaagctat cagctcttga gaagttaagt ttttctgaat tccaagcgtt tgcctcgaac 2100
cttttcaaca gtgtacatct tgaagttatg gtcttaggga acctttctga gcagcagaag 2160
aaagattatc ttgagatgct acaagttttc actgcgtcac gatcttcgca tgctacaaag 2220
cccttttatt acgagctaca gtctcaggaa atttctgaga tccatcatga ctatccgtta 2280
actgcaaacg ggatgctctt attacttcaa gataagagtt ccccgtctat acaagggaag 2340
gtttgtgcgg agatgctctt tgaatggttg catcacatta cttttgagga gcttagaacg 2400
caacagcaat tgggttatat ggtgggtgcg cgctatcgag agtttgcttc caggccgttt 2460
ggatttctct atatccgttc ggatgcgtat tctcctgaag agcttcttgc taaaacttct 2520
ctgttcctta acaaggtctc agcttctcct gagaaatttg gaatatcgca agagaaattc 2580
gcaaacatac gcaaagcgta tatcaataaa atcttggagc ctgagcattc cttagacatg 2640
atgaactcgg cgttattttc tctagcattt gagcggcctt ttgtggagtt ttcgactccc 2700
gacttgaaga ttgctattgc ggaaacgtta acatacgaag agttcttaaa atactgtcag 2760
tgtttcctta gcaatgaact agggacgcaa actagcgtct atatacgtgg cactcagaag 2820
acctct 2826
<210> SEQ ID NO 8
<211> LENGTH: 942
<212> TYPE: PRT
<213> ORGANISM: Chlamydia pneumoniae
<400> SEQUENCE: 8
Met Phe Trp Lys Leu Leu Cys Pro Ile Leu Ile Cys Thr Ser Leu Ser
1 5 10 15
Ile Thr Ser Cys Glu Gln Gln Phe Lys Val Val Pro Asn Gln Cys Pro
20 25 30
Leu Gln Val Ser Thr Pro Ala Ala Ala Asp Gln Lys Ile Glu Lys Ile
35 40 45
Ile Cys Ser Asn Gly Leu Pro Leu Leu Ile Ile Ser Asp Pro Asn Leu
50 55 60
Pro Thr Ser Gly Ala Ala Leu Leu Val Lys Thr Gly Asn Asn Ala Asp
65 70 75 80
Pro Glu Glu Tyr Pro Gly Met Ala His Phe Thr Glu His Cys Val Phe
85 90 95
Leu Gly Asn Glu Lys Tyr Pro Glu Val Ser Gly Phe Pro Gly Phe Leu
100 105 110
Ser Glu Asn Asn Gly Val His Asn Ala Phe Thr Tyr Pro Asn Lys Thr
115 120 125
Val Phe Val Phe Ser Val Glu His Ser Ala Phe Ser Asp Ala Leu Asp
130 135 140
Gln Phe Val His Leu Phe Ile Asn Pro Lys Phe Arg Gln Glu Asp Leu
145 150 155 160
Asp Arg Glu Lys Tyr Ala Val His Gln Glu Phe Ala Ala His Pro Leu
165 170 175
Ser Asp Gly Arg Arg Val His Arg Ile Gln Gln Leu Val Ala Pro Gln
180 185 190
Gly His Pro Cys Ala Arg Phe Gly Cys Gly Asn Ala Ser Thr Leu Thr
195 200 205
Pro Val Thr Thr Glu Lys Met Ala Glu Trp Phe Lys Leu His Tyr Ser
210 215 220
Pro Glu Asn Met Cys Ala Ile Ala Tyr Thr Ser Ala Pro Leu Ser Lys
225 230 235 240
Ala Lys Lys Gln Phe Ser Lys Ile Phe Ser Gln Ile Pro Arg Ser Lys
245 250 255
Asn Tyr Glu Arg Gln Glu Pro Phe Leu Pro Ser Gly Asp Thr Ser Ser
260 265 270
Leu Lys Asn Leu Tyr Ile Asn Gln Ala Ile Gln Pro Thr Ser Asn Leu
275 280 285
Glu Ile Tyr Trp His Ile Tyr Glu Ser Ser His Pro Ile Pro Leu Gly
290 295 300
Cys Tyr Lys Ala Leu Ala Glu Val Leu Arg Asn Glu Ser Lys Asn Ser
305 310 315 320
Leu Val Ser Leu Leu Lys Asn Glu Gln Leu Ile Thr Asp Leu Asp Val
325 330 335
Glu Phe Phe Arg Ser Ser Leu Asn Thr Gly Glu Phe Tyr Ile Ser Tyr
340 345 350
Glu Leu Thr Glu Lys Gly Asp Lys His Tyr Ser Gln Val Ile Asp Ser
355 360 365
Thr Phe Gln Tyr Leu Arg Tyr Ile Gln Glu His Gly Ile Pro Asn Tyr
370 375 380
Thr Leu Glu Glu Ile Ser Thr Ile Asn Ala Leu Asn Tyr Cys Tyr Ser
385 390 395 400
Ser Lys Ser Pro Leu Phe Asp Leu Leu Cys Lys Gln Ile Val Ser Leu
405 410 415
Gly Asn Glu Asp Leu Ser Thr Tyr Pro Tyr His Ser Leu Val Tyr Pro
420 425 430
Lys Tyr Ser Ser Glu Asp Glu Ser Ala Leu Leu Asn Leu Val Ser Asp
435 440 445
Pro Glu Gln Ala Arg Phe Val Leu Ser Ser Lys Asn Ser Glu His Trp
450 455 460
Glu Glu Ala Thr Gln Leu His Asp Pro Ile Phe Asp Met Thr Tyr Tyr
465 470 475 480
Val Lys Ala Leu Asp Gly Val Gln Asp Tyr Gly Lys Val Gln Ser Leu
485 490 495
Lys Pro Ile Ala Leu Pro Lys Pro Asn Leu Phe Ile Pro Lys Glu Val
500 505 510
Thr Leu Pro Gly Val His Leu Leu Lys Lys Gln Glu Phe Pro Phe Ala
515 520 525
Pro Ala Leu Ser Tyr Gln Asp Asp Lys Leu Thr Leu Tyr His Cys Glu
530 535 540
Asp His Tyr Tyr Thr Ala Pro Lys Leu Ser Ser Gln Ile Arg Ile Arg
545 550 555 560
Ser Pro Gln Ile Ser Arg Ser Ser Pro Gln Phe Leu Val Ala Thr Glu
565 570 575
Leu Tyr Cys Leu Ala Val Asn Asp Gln Leu Leu Arg Glu Tyr Tyr Pro
580 585 590
Ala Thr Gln Ala Gly Leu Ser Phe Thr Ser Ala Leu Gly Gly Asp Gly
595 600 605
Ile Asp Leu Arg Val Ser Gly Tyr Thr Thr Thr Val Pro Ala Leu Leu
610 615 620
Asn Ser Ile Leu Thr Ser Leu Pro Asn Leu Glu Ile Ser Tyr Glu Thr
625 630 635 640
Phe Leu Val Tyr Lys Lys Gln Leu Leu Glu Leu Tyr Gln Gly Ala Leu
645 650 655
Leu Asn Cys Pro Val Arg Ser Gly Leu Asp Glu Leu Ala Ser Gln Val
660 665 670
Met Lys Glu Thr Tyr Ser Asn Thr Thr Lys Leu Ser Ala Leu Glu Lys
675 680 685
Leu Ser Phe Ser Glu Phe Gln Ala Phe Ala Ser Asn Leu Phe Asn Ser
690 695 700
Val His Leu Glu Val Met Val Leu Gly Asn Leu Ser Glu Gln Gln Lys
705 710 715 720
Lys Asp Tyr Leu Glu Met Leu Gln Val Phe Thr Ala Ser Arg Ser Ser
725 730 735
His Ala Thr Lys Pro Phe Tyr Tyr Glu Leu Gln Ser Gln Glu Ile Ser
740 745 750
Glu Ile His His Asp Tyr Pro Leu Thr Ala Asn Gly Met Leu Leu Leu
755 760 765
Leu Gln Asp Lys Ser Ser Pro Ser Ile Gln Gly Lys Val Cys Ala Glu
770 775 780
Met Leu Phe Glu Trp Leu His His Ile Thr Phe Glu Glu Leu Arg Thr
785 790 795 800
Gln Gln Gln Leu Gly Tyr Met Val Gly Ala Arg Tyr Arg Glu Phe Ala
805 810 815
Ser Arg Pro Phe Gly Phe Leu Tyr Ile Arg Ser Asp Ala Tyr Ser Pro
820 825 830
Glu Glu Leu Leu Ala Lys Thr Ser Leu Phe Leu Asn Lys Val Ser Ala
835 840 845
Ser Pro Glu Lys Phe Gly Ile Ser Gln Glu Lys Phe Ala Asn Ile Arg
850 855 860
Lys Ala Tyr Ile Asn Lys Ile Leu Glu Pro Glu His Ser Leu Asp Met
865 870 875 880
Met Asn Ser Ala Leu Phe Ser Leu Ala Phe Glu Arg Pro Phe Val Glu
885 890 895
Phe Ser Thr Pro Asp Leu Lys Ile Ala Ile Ala Glu Thr Leu Thr Tyr
900 905 910
Glu Glu Phe Leu Lys Tyr Cys Gln Cys Phe Leu Ser Asn Glu Leu Gly
915 920 925
Thr Gln Thr Ser Val Tyr Ile Arg Gly Thr Gln Lys Thr Ser
930 935 940
<210> SEQ ID NO 9
<211> LENGTH: 831
<212> TYPE: DNA
<213> ORGANISM: Chlamydia pneumoniae
<400> SEQUENCE: 9
gtgactacag agaaaatggc agaatggttt aagctacatt attctcctga gaatatgtgt 60
gctattgctt acacatcagc tccgctctct aaagcaaaga aacagttctc aaagattttt 120
tctcagattc ctagatcaaa aaattatgaa agacaggaac cttttcttcc ttctggtgac 180
acctcgtcat taaagaatct ctatattaac caagcaattc agcctacctc taatctagaa 240
atttactggc atatttatga atcttcccat ccgattcctt taggctgtta caaggctctt 300
gctgaagttt taagaaatga gagtaagaac agtttagtct ctttattgaa aaacgagcag 360
ctaattacgg atttagacgt ggaattcttt agaagttctt taaatactgg agaattctat 420
attagctatg agcttacgga gaaaggcgat aaacactatt ctcaggttat tgatagtacc 480
ttccaatatc ttcgatatat tcaggaacac gggattccca actatacgtt agaagaaatt 540
tctacaatta atgctttaaa ctactgttac agttccaaaa gtccattgtt tgatctgctt 600
tgtaagcaga ttgtatcctt gggcaatgag gatctatcta cgtatcctta tcatagcctt 660
gtgtatccta aatactcttc tgaagacgag tctgctcttc ttaatttagt ctctgatcct 720
gaacaagcac gttttgtctt atctagtaag aactctgagc attgggaaga agcgactcag 780
ctccacgatc ctatttttga catgacctac tatgtaaaag ctctggacgg t 831
<210> SEQ ID NO 10
<211> LENGTH: 277
<212> TYPE: PRT
<213> ORGANISM: Chlamydia pneumoniae
<400> SEQUENCE: 10
Val Thr Thr Glu Lys Met Ala Glu Trp Phe Lys Leu His Tyr Ser Pro
1 5 10 15
Glu Asn Met Cys Ala Ile Ala Tyr Thr Ser Ala Pro Leu Ser Lys Ala
20 25 30
Lys Lys Gln Phe Ser Lys Ile Phe Ser Gln Ile Pro Arg Ser Lys Asn
35 40 45
Tyr Glu Arg Gln Glu Pro Phe Leu Pro Ser Gly Asp Thr Ser Ser Leu
50 55 60
Lys Asn Leu Tyr Ile Asn Gln Ala Ile Gln Pro Thr Ser Asn Leu Glu
65 70 75 80
Ile Tyr Trp His Ile Tyr Glu Ser Ser His Pro Ile Pro Leu Gly Cys
85 90 95
Tyr Lys Ala Leu Ala Glu Val Leu Arg Asn Glu Ser Lys Asn Ser Leu
100 105 110
Val Ser Leu Leu Lys Asn Glu Gln Leu Ile Thr Asp Leu Asp Val Glu
115 120 125
Phe Phe Arg Ser Ser Leu Asn Thr Gly Glu Phe Tyr Ile Ser Tyr Glu
130 135 140
Leu Thr Glu Lys Gly Asp Lys His Tyr Ser Gln Val Ile Asp Ser Thr
145 150 155 160
Phe Gln Tyr Leu Arg Tyr Ile Gln Glu His Gly Ile Pro Asn Tyr Thr
165 170 175
Leu Glu Glu Ile Ser Thr Ile Asn Ala Leu Asn Tyr Cys Tyr Ser Ser
180 185 190
Lys Ser Pro Leu Phe Asp Leu Leu Cys Lys Gln Ile Val Ser Leu Gly
195 200 205
Asn Glu Asp Leu Ser Thr Tyr Pro Tyr His Ser Leu Val Tyr Pro Lys
210 215 220
Tyr Ser Ser Glu Asp Glu Ser Ala Leu Leu Asn Leu Val Ser Asp Pro
225 230 235 240
Glu Gln Ala Arg Phe Val Leu Ser Ser Lys Asn Ser Glu His Trp Glu
245 250 255
Glu Ala Thr Gln Leu His Asp Pro Ile Phe Asp Met Thr Tyr Tyr Val
260 265 270
Lys Ala Leu Asp Gly
275
<210> SEQ ID NO 11
<211> LENGTH: 1458
<212> TYPE: DNA
<213> ORGANISM: Chlamydia pneumoniae
<400> SEQUENCE: 11
atgttttgga aacttttatg tcctatttta atttgcactt ccctatccat aacatcgtgt 60
gaacagcagt tcaaagtcgt ccccaatcag tgccctttac aagtttccac tcctgccgct 120
gcggaccaga aaattgaaaa gattatttgt agtaacgggc tccctcttct tattatttcc 180
gaccctaatc ttcctacttc gggagcagca ctccttgtga aaacaggaaa taatgccgat 240
cctgaagagt atcctgggat ggcgcacttc acagaacact gtgtctttct tggaaatgaa 300
aagtatcctg aggtctctgg tttccctgga tttttaagcg aaaataatgg ggtgcataat 360
gctttcactt acccaaataa aacagtcttt gtattttcag tagaacattc tgcgttttct 420
gatgctttag accaatttgt tcatctattt attaatccga agtttcgtca agaagatctt 480
gatagagaaa agtacgcagt acatcaagaa ttcgctgctc atcctctttc tgatgggaga 540
cgtgtgcatc gcattcagca gcttgttgct cctcagggcc atccctgcgc acgttttggt 600
tgtgggaatg cttccaccct caccccagtg actacagaga aaatggcaga atggtttaag 660
ctacattatt ctcctgagaa tatgtgtgct attgcttaca catcagctcc gctctctaaa 720
gcaaagaaac agttctcaaa gattttttct cagattccta gatcaaaaaa ttatgaaaga 780
caggaacctt ttcttccttc tggtgacacc tcgtcattaa agaatctcta tattaaccaa 840
gcaattcagc ctacctctaa tctagaaatt tactggcata tttatgaatc ttcccatccg 900
attcctttag gctgttacaa ggctcttgct gaagttttaa gaaatgagag taagaacagt 960
ttagtctctt tattgaaaaa cgagcagcta attacggatt tagacgtgga attctttaga 1020
agttctttaa atactggaga attctatatt agctatgagc ttacggagaa aggcgataaa 1080
cactattctc aggttattga tagtaccttc caatatcttc gatatattca ggaacacggg 1140
attcccaact atacgttaga agaaatttct acaattaatg ctttaaacta ctgttacagt 1200
tccaaaagtc cattgtttga tctgctttgt aagcagattg tatccttggg caatgaggat 1260
ctatctacgt atccttatca tagccttgtg tatcctaaat actcttctga agacgagtct 1320
gctcttctta atttagtctc tgatcctgaa caagcacgtt ttgtcttatc tagtaagaac 1380
tctgagcatt gggaagaagc gactcagctc cacgatccta tttttgacat gacctactat 1440
gtaaaagctc tggacggt 1458
<210> SEQ ID NO 12
<211> LENGTH: 486
<212> TYPE: PRT
<213> ORGANISM: Chlamydia pneumoniae
<400> SEQUENCE: 12
Met Phe Trp Lys Leu Leu Cys Pro Ile Leu Ile Cys Thr Ser Leu Ser
1 5 10 15
Ile Thr Ser Cys Glu Gln Gln Phe Lys Val Val Pro Asn Gln Cys Pro
20 25 30
Leu Gln Val Ser Thr Pro Ala Ala Ala Asp Gln Lys Ile Glu Lys Ile
35 40 45
Ile Cys Ser Asn Gly Leu Pro Leu Leu Ile Ile Ser Asp Pro Asn Leu
50 55 60
Pro Thr Ser Gly Ala Ala Leu Leu Val Lys Thr Gly Asn Asn Ala Asp
65 70 75 80
Pro Glu Glu Tyr Pro Gly Met Ala His Phe Thr Glu His Cys Val Phe
85 90 95
Leu Gly Asn Glu Lys Tyr Pro Glu Val Ser Gly Phe Pro Gly Phe Leu
100 105 110
Ser Glu Asn Asn Gly Val His Asn Ala Phe Thr Tyr Pro Asn Lys Thr
115 120 125
Val Phe Val Phe Ser Val Glu His Ser Ala Phe Ser Asp Ala Leu Asp
130 135 140
Gln Phe Val His Leu Phe Ile Asn Pro Lys Phe Arg Gln Glu Asp Leu
145 150 155 160
Asp Arg Glu Lys Tyr Ala Val His Gln Glu Phe Ala Ala His Pro Leu
165 170 175
Ser Asp Gly Arg Arg Val His Arg Ile Gln Gln Leu Val Ala Pro Gln
180 185 190
Gly His Pro Cys Ala Arg Phe Gly Cys Gly Asn Ala Ser Thr Leu Thr
195 200 205
Pro Val Thr Thr Glu Lys Met Ala Glu Trp Phe Lys Leu His Tyr Ser
210 215 220
Pro Glu Asn Met Cys Ala Ile Ala Tyr Thr Ser Ala Pro Leu Ser Lys
225 230 235 240
Ala Lys Lys Gln Phe Ser Lys Ile Phe Ser Gln Ile Pro Arg Ser Lys
245 250 255
Asn Tyr Glu Arg Gln Glu Pro Phe Leu Pro Ser Gly Asp Thr Ser Ser
260 265 270
Leu Lys Asn Leu Tyr Ile Asn Gln Ala Ile Gln Pro Thr Ser Asn Leu
275 280 285
Glu Ile Tyr Trp His Ile Tyr Glu Ser Ser His Pro Ile Pro Leu Gly
290 295 300
Cys Tyr Lys Ala Leu Ala Glu Val Leu Arg Asn Glu Ser Lys Asn Ser
305 310 315 320
Leu Val Ser Leu Leu Lys Asn Glu Gln Leu Ile Thr Asp Leu Asp Val
325 330 335
Glu Phe Phe Arg Ser Ser Leu Asn Thr Gly Glu Phe Tyr Ile Ser Tyr
340 345 350
Glu Leu Thr Glu Lys Gly Asp Lys His Tyr Ser Gln Val Ile Asp Ser
355 360 365
Thr Phe Gln Tyr Leu Arg Tyr Ile Gln Glu His Gly Ile Pro Asn Tyr
370 375 380
Thr Leu Glu Glu Ile Ser Thr Ile Asn Ala Leu Asn Tyr Cys Tyr Ser
385 390 395 400
Ser Lys Ser Pro Leu Phe Asp Leu Leu Cys Lys Gln Ile Val Ser Leu
405 410 415
Gly Asn Glu Asp Leu Ser Thr Tyr Pro Tyr His Ser Leu Val Tyr Pro
420 425 430
Lys Tyr Ser Ser Glu Asp Glu Ser Ala Leu Leu Asn Leu Val Ser Asp
435 440 445
Pro Glu Gln Ala Arg Phe Val Leu Ser Ser Lys Asn Ser Glu His Trp
450 455 460
Glu Glu Ala Thr Gln Leu His Asp Pro Ile Phe Asp Met Thr Tyr Tyr
465 470 475 480
Val Lys Ala Leu Asp Gly
485
<210> SEQ ID NO 13
<211> LENGTH: 2757
<212> TYPE: DNA
<213> ORGANISM: Chlamydia pneumoniae
<400> SEQUENCE: 13
atgggtgaag tctatcttgc ctacgatcct gtatgttctc gtaaagtagc tcttaaaaaa 60
attcgtgaag atcttgcaga aaatcctctt ttgaaaagga ggtttttacg agaggcaaga 120
attgccgctg accttattca tcctggtgtt gttcctgtct atactattta cagcgagaaa 180
gatcctgtat actacacgat gccctacata gagggatata cactaaaaac cttactgaag 240
agtgtatggc aaaaggaatc cctgtctaag gaattagcag agaaaacttc tgtaggggca 300
tttctttcta tctttcataa gatctgctgc actatagaat atgtccattc tcggggcatt 360
cttcatcgcg accttaaacc cgataacatc ttattaggtc tttttagtga ggctgtaatc 420
ttagattggg gagcagcagt tgcctgtgga gaagaagagg atcttcttga tatagatgtc 480
agcaaagagg aggtgctctc ttcaagaatg acaattccag gaagaatagt agggactcca 540
gattatatgg ctcctgagag gctcctgggc catccagctt ctaaaagtac agacatttat 600
gctttaggag tggttcttta tcagatgctc actctctctt ttccttatag aagaaaaaaa 660
ggaaagaaaa tagttcttga cggtcagaga attccaagtc ctcaagaggt agctccttat 720
cgagaaatcc ctccgtttct ttccgctgta gtgatgagaa tgttggctgt agatcctcaa 780
gagcgctatt cttcggtaac agagcttaag gaagatatcg agagtcatct gaaagggagt 840
cctaaatgga ctttaaccac agccctgcca cctaaaaaat cttctagttg gaagctaaac 900
gaacctattt tactttctaa gtattttcca atgttggagg tctctccagc gtcatggtac 960
agtttagcaa tctctaatat tgagagtttt tctgagatgc gcttggagta tactctttct 1020
aaaaaaggct tgaacgaagg ctttggtatt ttacttccca cgtcagaaaa tgctttaggg 1080
ggagattttt accaggggta tggcttttgg ctgcatatta aggagagaac cttatccgtg 1140
tctctggtga aaaatagcct agaaatccag aggtgctctc aagatttgga atctgataaa 1200
gagaccttct tgatagcttt agagcagcat aatcatagtt tatctttgtt tgtcgatggt 1260
acgacttggc ttatccatat gaattatctg ccaagtcgta gtgggcgagt cgctatcata 1320
gttcgcgata tggaagatat cctggaagat ataggcattt ttgaaagtag tggctctttg 1380
agggtcagtt gtcttgctgt tcctgacgct tttcttgctg agaagttata tgatcgcgct 1440
ttagtgcttt accgaaggat cgcagaatct ttcccaggac gtaaagaagg ttatgaagca 1500
aggttcagag caggaattac agttttagag aaggcctcta cagataataa tgaacaggaa 1560
tttgctctag ccattgaaga attctcaaaa ttacatgacg gggttgctgc tcccttagaa 1620
taccttggta aggctttagt atatcagaga ctccaagagt ataatgaaga aattaagagt 1680
ttgctattag cattgaaacg ttattcgcag catcctgaaa tctttaggct taaagaccat 1740
gtggtttacc gactccatga gagcttttat aaacgggatc gccttgctct ggtgttcatg 1800
attttagtat tggaaatagc tccccaggca atcactccag ggcaggaaga aaaaatcctg 1860
gtttggttaa aggacaaatc tcgggctacc ttattttgcc tcctggatcc cacggtctta 1920
gagctgcgct cttctaaaat ggaattattt ttaagttatt ggtctgggtt tattccccat 1980
ctcaatagtc tatttcatag agcttgggat caaagcgatg tgcgagcttt gatcgagatt 2040
ttctatgttg cttgtgatct tcataaatgg cagtttctct cttcttgtat cgacatattt 2100
aaagagtctc ttgaggatca gaaagccaca gaagagattg ttgagttctc tttcgaggat 2160
ttaggggcat ttctttttgc tattcagagc atctttaaca aggaagatgc agagaagatc 2220
tttgtttcta atgatcaatt atcgccaatc cttcttgttt atatattcga tctttttgca 2280
aatcgtgctc ttctggaatc tcaaggagag gctatttttc aggctttgga tctcatccga 2340
agtaaagttc ctgaaaattt ttatcatgat tacttgcgga atcatgaaat ccgagcgcat 2400
ctttggtgcc gcaatgagaa ggctctaagc acgatttttg aaaactatac agagaaacag 2460
ctaaaggatg agcaacatga actgttcgtt ctctatggat gttaccttgc tcttatacaa 2520
ggtgctgagg cggcaaagca gcattttgat gtatgtcgtg aagatcgcat tttccctgct 2580
tcattattag ctagaaatta caatcgttta ggtcttccca aagatgctct tagctatcaa 2640
gagcggcgtt tgttattgcg acaaaagttt ctctatttcc attgtcttgg taaccacgac 2700
gagcgtgact tatgccagac tatgtatcac ctcttaaccg aagaatttca gctttaa 2757
<210> SEQ ID NO 14
<211> LENGTH: 918
<212> TYPE: PRT
<213> ORGANISM: Chlamydia pneumoniae
<400> SEQUENCE: 14
Met Gly Glu Val Tyr Leu Ala Tyr Asp Pro Val Cys Ser Arg Lys Val
1 5 10 15
Ala Leu Lys Lys Ile Arg Glu Asp Leu Ala Glu Asn Pro Leu Leu Lys
20 25 30
Arg Arg Phe Leu Arg Glu Ala Arg Ile Ala Ala Asp Leu Ile His Pro
35 40 45
Gly Val Val Pro Val Tyr Thr Ile Tyr Ser Glu Lys Asp Pro Val Tyr
50 55 60
Tyr Thr Met Pro Tyr Ile Glu Gly Tyr Thr Leu Lys Thr Leu Leu Lys
65 70 75 80
Ser Val Trp Gln Lys Glu Ser Leu Ser Lys Glu Leu Ala Glu Lys Thr
85 90 95
Ser Val Gly Ala Phe Leu Ser Ile Phe His Lys Ile Cys Cys Thr Ile
100 105 110
Glu Tyr Val His Ser Arg Gly Ile Leu His Arg Asp Leu Lys Pro Asp
115 120 125
Asn Ile Leu Leu Gly Leu Phe Ser Glu Ala Val Ile Leu Asp Trp Gly
130 135 140
Ala Ala Val Ala Cys Gly Glu Glu Glu Asp Leu Leu Asp Ile Asp Val
145 150 155 160
Ser Lys Glu Glu Val Leu Ser Ser Arg Met Thr Ile Pro Gly Arg Ile
165 170 175
Val Gly Thr Pro Asp Tyr Met Ala Pro Glu Arg Leu Leu Gly His Pro
180 185 190
Ala Ser Lys Ser Thr Asp Ile Tyr Ala Leu Gly Val Val Leu Tyr Gln
195 200 205
Met Leu Thr Leu Ser Phe Pro Tyr Arg Arg Lys Lys Gly Lys Lys Ile
210 215 220
Val Leu Asp Gly Gln Arg Ile Pro Ser Pro Gln Glu Val Ala Pro Tyr
225 230 235 240
Arg Glu Ile Pro Pro Phe Leu Ser Ala Val Val Met Arg Met Leu Ala
245 250 255
Val Asp Pro Gln Glu Arg Tyr Ser Ser Val Thr Glu Leu Lys Glu Asp
260 265 270
Ile Glu Ser His Leu Lys Gly Ser Pro Lys Trp Thr Leu Thr Thr Ala
275 280 285
Leu Pro Pro Lys Lys Ser Ser Ser Trp Lys Leu Asn Glu Pro Ile Leu
290 295 300
Leu Ser Lys Tyr Phe Pro Met Leu Glu Val Ser Pro Ala Ser Trp Tyr
305 310 315 320
Ser Leu Ala Ile Ser Asn Ile Glu Ser Phe Ser Glu Met Arg Leu Glu
325 330 335
Tyr Thr Leu Ser Lys Lys Gly Leu Asn Glu Gly Phe Gly Ile Leu Leu
340 345 350
Pro Thr Ser Glu Asn Ala Leu Gly Gly Asp Phe Tyr Gln Gly Tyr Gly
355 360 365
Phe Trp Leu His Ile Lys Glu Arg Thr Leu Ser Val Ser Leu Val Lys
370 375 380
Asn Ser Leu Glu Ile Gln Arg Cys Ser Gln Asp Leu Glu Ser Asp Lys
385 390 395 400
Glu Thr Phe Leu Ile Ala Leu Glu Gln His Asn His Ser Leu Ser Leu
405 410 415
Phe Val Asp Gly Thr Thr Trp Leu Ile His Met Asn Tyr Leu Pro Ser
420 425 430
Arg Ser Gly Arg Val Ala Ile Ile Val Arg Asp Met Glu Asp Ile Leu
435 440 445
Glu Asp Ile Gly Ile Phe Glu Ser Ser Gly Ser Leu Arg Val Ser Cys
450 455 460
Leu Ala Val Pro Asp Ala Phe Leu Ala Glu Lys Leu Tyr Asp Arg Ala
465 470 475 480
Leu Val Leu Tyr Arg Arg Ile Ala Glu Ser Phe Pro Gly Arg Lys Glu
485 490 495
Gly Tyr Glu Ala Arg Phe Arg Ala Gly Ile Thr Val Leu Glu Lys Ala
500 505 510
Ser Thr Asp Asn Asn Glu Gln Glu Phe Ala Leu Ala Ile Glu Glu Phe
515 520 525
Ser Lys Leu His Asp Gly Val Ala Ala Pro Leu Glu Tyr Leu Gly Lys
530 535 540
Ala Leu Val Tyr Gln Arg Leu Gln Glu Tyr Asn Glu Glu Ile Lys Ser
545 550 555 560
Leu Leu Leu Ala Leu Lys Arg Tyr Ser Gln His Pro Glu Ile Phe Arg
565 570 575
Leu Lys Asp His Val Val Tyr Arg Leu His Glu Ser Phe Tyr Lys Arg
580 585 590
Asp Arg Leu Ala Leu Val Phe Met Ile Leu Val Leu Glu Ile Ala Pro
595 600 605
Gln Ala Ile Thr Pro Gly Gln Glu Glu Lys Ile Leu Val Trp Leu Lys
610 615 620
Asp Lys Ser Arg Ala Thr Leu Phe Cys Leu Leu Asp Pro Thr Val Leu
625 630 635 640
Glu Leu Arg Ser Ser Lys Met Glu Leu Phe Leu Ser Tyr Trp Ser Gly
645 650 655
Phe Ile Pro His Leu Asn Ser Leu Phe His Arg Ala Trp Asp Gln Ser
660 665 670
Asp Val Arg Ala Leu Ile Glu Ile Phe Tyr Val Ala Cys Asp Leu His
675 680 685
Lys Trp Gln Phe Leu Ser Ser Cys Ile Asp Ile Phe Lys Glu Ser Leu
690 695 700
Glu Asp Gln Lys Ala Thr Glu Glu Ile Val Glu Phe Ser Phe Glu Asp
705 710 715 720
Leu Gly Ala Phe Leu Phe Ala Ile Gln Ser Ile Phe Asn Lys Glu Asp
725 730 735
Ala Glu Lys Ile Phe Val Ser Asn Asp Gln Leu Ser Pro Ile Leu Leu
740 745 750
Val Tyr Ile Phe Asp Leu Phe Ala Asn Arg Ala Leu Leu Glu Ser Gln
755 760 765
Gly Glu Ala Ile Phe Gln Ala Leu Asp Leu Ile Arg Ser Lys Val Pro
770 775 780
Glu Asn Phe Tyr His Asp Tyr Leu Arg Asn His Glu Ile Arg Ala His
785 790 795 800
Leu Trp Cys Arg Asn Glu Lys Ala Leu Ser Thr Ile Phe Glu Asn Tyr
805 810 815
Thr Glu Lys Gln Leu Lys Asp Glu Gln His Glu Leu Phe Val Leu Tyr
820 825 830
Gly Cys Tyr Leu Ala Leu Ile Gln Gly Ala Glu Ala Ala Lys Gln His
835 840 845
Phe Asp Val Cys Arg Glu Asp Arg Ile Phe Pro Ala Ser Leu Leu Ala
850 855 860
Arg Asn Tyr Asn Arg Leu Gly Leu Pro Lys Asp Ala Leu Ser Tyr Gln
865 870 875 880
Glu Arg Arg Leu Leu Leu Arg Gln Lys Phe Leu Tyr Phe His Cys Leu
885 890 895
Gly Asn His Asp Glu Arg Asp Leu Cys Gln Thr Met Tyr His Leu Leu
900 905 910
Thr Glu Glu Phe Gln Leu
915
<210> SEQ ID NO 15
<211> LENGTH: 984
<212> TYPE: DNA
<213> ORGANISM: Chlamydia pneumoniae
<400> SEQUENCE: 15
atgggtgaag tctatcttgc ctacgatcct gtatgttctc gtaaagtagc tcttaaaaaa 60
attcgtgaag atcttgcaga aaatcctctt ttgaaaagga ggtttttacg agaggcaaga 120
attgccgctg accttattca tcctggtgtt gttcctgtct atactattta cagcgagaaa 180
gatcctgtat actacacgat gccctacata gagggatata cactaaaaac cttactgaag 240
agtgtatggc aaaaggaatc cctgtctaag gaattagcag agaaaacttc tgtaggggca 300
tttctttcta tctttcataa gatctgctgc actatagaat atgtccattc tcggggcatt 360
cttcatcgcg accttaaacc cgataacatc ttattaggtc tttttagtga ggctgtaatc 420
ttagattggg gagcagcagt tgcctgtgga gaagaagagg atcttcttga tatagatgtc 480
agcaaagagg aggtgctctc ttcaagaatg acaattccag gaagaatagt agggactcca 540
gattatatgg ctcctgagag gctcctgggc catccagctt ctaaaagtac agacatttat 600
gctttaggag tggttcttta tcagatgctc actctctctt ttccttatag aagaaaaaaa 660
ggaaagaaaa tagttcttga cggtcagaga attccaagtc ctcaagaggt agctccttat 720
cgagaaatcc ctccgtttct ttccgctgta gtgatgagaa tgttggctgt agatcctcaa 780
gagcgctatt cttcggtaac agagcttaag gaagatatcg agagtcatct gaaagggagt 840
cctaaatgga ctttaaccac agccctgcca cctaaaaaat cttctagttg gaagctaaac 900
gaacctattt tactttctaa gtattttcca atgttggagg tctctccagc gtcatggtac 960
agtttagcaa tctctaatat tgag 984
<210> SEQ ID NO 16
<211> LENGTH: 329
<212> TYPE: PRT
<213> ORGANISM: Chlamydia pneumoniae
<400> SEQUENCE: 16
Met Gly Glu Val Tyr Leu Ala Tyr Asp Pro Val Cys Ser Arg Lys Val
1 5 10 15
Ala Leu Lys Lys Ile Arg Glu Asp Leu Ala Glu Asn Pro Leu Leu Lys
20 25 30
Arg Arg Phe Leu Arg Glu Ala Arg Ile Ala Ala Asp Leu Ile His Pro
35 40 45
Gly Val Val Pro Val Tyr Thr Ile Tyr Ser Glu Lys Asp Pro Val Tyr
50 55 60
Tyr Thr Met Pro Tyr Ile Glu Gly Tyr Thr Leu Lys Thr Leu Leu Lys
65 70 75 80
Ser Val Trp Gln Lys Glu Ser Leu Ser Lys Glu Leu Ala Glu Lys Thr
85 90 95
Ser Val Gly Ala Phe Leu Ser Ile Phe His Lys Ile Cys Cys Thr Ile
100 105 110
Glu Tyr Val His Ser Arg Gly Ile Leu His Arg Asp Leu Lys Pro Asp
115 120 125
Asn Ile Leu Leu Gly Leu Phe Ser Glu Ala Val Ile Leu Asp Trp Gly
130 135 140
Ala Ala Val Ala Cys Gly Glu Glu Glu Asp Leu Leu Asp Ile Asp Val
145 150 155 160
Ser Lys Glu Glu Val Leu Ser Ser Arg Met Thr Ile Pro Gly Arg Ile
165 170 175
Val Gly Thr Pro Asp Tyr Met Ala Pro Glu Arg Leu Leu Gly His Pro
180 185 190
Ala Ser Lys Ser Thr Asp Ile Tyr Ala Leu Gly Val Val Leu Tyr Gln
195 200 205
Met Leu Thr Leu Ser Phe Pro Tyr Arg Arg Lys Lys Gly Lys Lys Ile
210 215 220
Val Leu Asp Gly Gln Arg Ile Pro Ser Pro Gln Glu Val Ala Pro Tyr
225 230 235 240
Arg Glu Ile Pro Pro Phe Leu Ser Ala Val Val Met Arg Met Leu Ala
245 250 255
Val Asp Pro Gln Glu Arg Tyr Ser Ser Val Thr Glu Leu Lys Glu Asp
260 265 270
Ile Glu Ser His Leu Lys Gly Ser Pro Lys Trp Thr Leu Thr Thr Ala
275 280 285
Leu Pro Pro Lys Lys Ser Ser Ser Trp Lys Leu Asn Glu Pro Ile Leu
290 295 300
Leu Ser Lys Tyr Phe Pro Met Leu Glu Val Ser Pro Ala Ser Trp Tyr
305 310 315 320
Ser Leu Ala Ile Ser Asn Ile Glu Thr
325
<210> SEQ ID NO 17
<211> LENGTH: 1575
<212> TYPE: DNA
<213> ORGANISM: Chlamydia pneumoniae
<400> SEQUENCE: 17
atgctccgtt tcttcgctgt atttatatca actctttggc tcattacctc aggatgttcc 60
ccatcccaat cctctaaagg aatttttgtg gtaaatatga aggaaatgcc acgctccttg 120
gatcctggaa aaactcgtct cattgcagac caaactctaa tgcgtcatct atatgaagga 180
ctcgtcgaag aacattccca aaatggagag attaaaccag cccttgcaga aagctacacc 240
atctccgaag acgggactcg gtacacattt aaaatcaaaa acatcctttg gagtaacgga 300
gaccctctga cagctcaaga ctttgtctcc tcttggaagg aaatcctaaa ggaagatgcg 360
tcctccgtat atctctatgc gtttttacct atcaaaaatg ctcgggcaat ctttgatgat 420
actgagtctc cagaaaatct aggagtccga gctttagata agcgtcatct cgaaattcag 480
ttagaaactc cctgcgcgca tttcctacat ttcttgactc ttcctatttt tttccctgtt 540
catgaaactc tgcgaaacta tagcacctct tttgaagaga tgcccattac ctgcggtgct 600
ttccgccctg tgtctctaga aaaaggcctg agactccatc tagagaaaaa ccctatgtac 660
cataataaaa gccgtgtgaa actacataaa attattgtac agtttatctc aaacgctaac 720
actgcagcca ttctattcaa acataagaaa ttagattggc aaggacctcc ttggggagaa 780
cctatccctc cagaaatctc agcttctcta catcaagatg accagctctt ttctcttccg 840
ggcgcttcga ctacatggtt actctttaat atacaaaaaa aaccttggaa caatgctaaa 900
ttacgcaagg cattgagcct tgcaatagac aaagatatgt taaccaaagt ggtataccaa 960
ggtcttgcag aacctacaga tcatatccta catccaagac tttatccagg gacctatccc 1020
gaacggaaaa gacaaaacga aagaattctt gaggctcaac aactctttga agaagctcta 1080
gacgaacttc aaatgacacg cgaagatcta gaaaaggaaa ctttgacttt ctcaaccttt 1140
tctttttctt acggaaggat ttgccaaatg ctaagagaac aatggaagaa agtcttaaaa 1200
tttactatcc ctatagtagg ccaagagttt ttcacaatac aaaaaaactt cctagagggg 1260
aactattccc taaccgtgaa ccaatggacc gcagcattta ttgatccgat gtcttatctc 1320
atgatctttg ccaatcctgg aggaatttcc ccctatcacc tccaagattc acactttcaa 1380
actcttctca taaagatcac tcaagaacat aaaaaacacc tacgaaatca gcttattatt 1440
gaagcccttg actatttaga acactgtcac attctcgaac cactatgtca tccaaatctt 1500
cgaattgctt tgaacaaaaa cattaaaaac tttaatcttt ttgttcgacg aacttcagac 1560
tttcgtttta tagaa 1575
<210> SEQ ID NO 18
<211> LENGTH: 525
<212> TYPE: PRT
<213> ORGANISM: Chlamydia pneumoniae
<400> SEQUENCE: 18
Met Leu Arg Phe Phe Ala Val Phe Ile Ser Thr Leu Trp Leu Ile Thr
1 5 10 15
Ser Gly Cys Ser Pro Ser Gln Ser Ser Lys Gly Ile Phe Val Val Asn
20 25 30
Met Lys Glu Met Pro Arg Ser Leu Asp Pro Gly Lys Thr Arg Leu Ile
35 40 45
Ala Asp Gln Thr Leu Met Arg His Leu Tyr Glu Gly Leu Val Glu Glu
50 55 60
His Ser Gln Asn Gly Glu Ile Lys Pro Ala Leu Ala Glu Ser Tyr Thr
65 70 75 80
Ile Ser Glu Asp Gly Thr Arg Tyr Thr Phe Lys Ile Lys Asn Ile Leu
85 90 95
Trp Ser Asn Gly Asp Pro Leu Thr Ala Gln Asp Phe Val Ser Ser Trp
100 105 110
Lys Glu Ile Leu Lys Glu Asp Ala Ser Ser Val Tyr Leu Tyr Ala Phe
115 120 125
Leu Pro Ile Lys Asn Ala Arg Ala Ile Phe Asp Asp Thr Glu Ser Pro
130 135 140
Glu Asn Leu Gly Val Arg Ala Leu Asp Lys Arg His Leu Glu Ile Gln
145 150 155 160
Leu Glu Thr Pro Cys Ala His Phe Leu His Phe Leu Thr Leu Pro Ile
165 170 175
Phe Phe Pro Val His Glu Thr Leu Arg Asn Tyr Ser Thr Ser Phe Glu
180 185 190
Glu Met Pro Ile Thr Cys Gly Ala Phe Arg Pro Val Ser Leu Glu Lys
195 200 205
Gly Leu Arg Leu His Leu Glu Lys Asn Pro Met Tyr His Asn Lys Ser
210 215 220
Arg Val Lys Leu His Lys Ile Ile Val Gln Phe Ile Ser Asn Ala Asn
225 230 235 240
Thr Ala Ala Ile Leu Phe Lys His Lys Lys Leu Asp Trp Gln Gly Pro
245 250 255
Pro Trp Gly Glu Pro Ile Pro Pro Glu Ile Ser Ala Ser Leu His Gln
260 265 270
Asp Asp Gln Leu Phe Ser Leu Pro Gly Ala Ser Thr Thr Trp Leu Leu
275 280 285
Phe Asn Ile Gln Lys Lys Pro Trp Asn Asn Ala Lys Leu Arg Lys Ala
290 295 300
Leu Ser Leu Ala Ile Asp Lys Asp Met Leu Thr Lys Val Val Tyr Gln
305 310 315 320
Gly Leu Ala Glu Pro Thr Asp His Ile Leu His Pro Arg Leu Tyr Pro
325 330 335
Gly Thr Tyr Pro Glu Arg Lys Arg Gln Asn Glu Arg Ile Leu Glu Ala
340 345 350
Gln Gln Leu Phe Glu Glu Ala Leu Asp Glu Leu Gln Met Thr Arg Glu
355 360 365
Asp Leu Glu Lys Glu Thr Leu Thr Phe Ser Thr Phe Ser Phe Ser Tyr
370 375 380
Gly Arg Ile Cys Gln Met Leu Arg Glu Gln Trp Lys Lys Val Leu Lys
385 390 395 400
Phe Thr Ile Pro Ile Val Gly Gln Glu Phe Phe Thr Ile Gln Lys Asn
405 410 415
Phe Leu Glu Gly Asn Tyr Ser Leu Thr Val Asn Gln Trp Thr Ala Ala
420 425 430
Phe Ile Asp Pro Met Ser Tyr Leu Met Ile Phe Ala Asn Pro Gly Gly
435 440 445
Ile Ser Pro Tyr His Leu Gln Asp Ser His Phe Gln Thr Leu Leu Ile
450 455 460
Lys Ile Thr Gln Glu His Lys Lys His Leu Arg Asn Gln Leu Ile Ile
465 470 475 480
Glu Ala Leu Asp Tyr Leu Glu His Cys His Ile Leu Glu Pro Leu Cys
485 490 495
His Pro Asn Leu Arg Ile Ala Leu Asn Lys Asn Ile Lys Asn Phe Asn
500 505 510
Leu Phe Val Arg Arg Thr Ser Asp Phe Arg Phe Ile Glu
515 520 525
<210> SEQ ID NO 19
<211> LENGTH: 864
<212> TYPE: DNA
<213> ORGANISM: Chlamydia pneumoniae
<400> SEQUENCE: 19
atgctccgtt tcttcgctgt atttatatca actctttggc tcattacctc aggatgttcc 60
ccatcccaat cctctaaagg aatttttgtg gtaaatatga aggaaatgcc acgctccttg 120
gatcctggaa aaactcgtct cattgcagac caaactctaa tgcgtcatct atatgaagga 180
ctcgtcgaag aacattccca aaatggagag attaaaccag cccttgcaga aagctacacc 240
atctccgaag acgggactcg gtacacattt aaaatcaaaa acatcctttg gagtaacgga 300
gaccctctga cagctcaaga ctttgtctcc tcttggaagg aaatcctaaa ggaagatgcg 360
tcctccgtat atctctatgc gtttttacct atcaaaaatg ctcgggcaat ctttgatgat 420
actgagtctc cagaaaatct aggagtccga gctttagata agcgtcatct cgaaattcag 480
ttagaaactc cctgcgcgca tttcctacat ttcttgactc ttcctatttt tttccctgtt 540
catgaaactc tgcgaaacta tagcacctct tttgaagaga tgcccattac ctgcggtgct 600
ttccgccctg tgtctctaga aaaaggcctg agactccatc tagagaaaaa ccctatgtac 660
cataataaaa gccgtgtgaa actacataaa attattgtac agtttatctc aaacgctaac 720
actgcagcca ttctattcaa acataagaaa ttagattggc aaggacctcc ttggggagaa 780
cctatccctc cagaaatctc agcttctcta catcaagatg accagctctt ttctcttccg 840
ggcgcttcga ctacatggtt actc 864
<210> SEQ ID NO 20
<211> LENGTH: 288
<212> TYPE: PRT
<213> ORGANISM: Chlamydia pneumoniae
<400> SEQUENCE: 20
Met Leu Arg Phe Phe Ala Val Phe Ile Ser Thr Leu Trp Leu Ile Thr
1 5 10 15
Ser Gly Cys Ser Pro Ser Gln Ser Ser Lys Gly Ile Phe Val Val Asn
20 25 30
Met Lys Glu Met Pro Arg Ser Leu Asp Pro Gly Lys Thr Arg Leu Ile
35 40 45
Ala Asp Gln Thr Leu Met Arg His Leu Tyr Glu Gly Leu Val Glu Glu
50 55 60
His Ser Gln Asn Gly Glu Ile Lys Pro Ala Leu Ala Glu Ser Tyr Thr
65 70 75 80
Ile Ser Glu Asp Gly Thr Arg Tyr Thr Phe Lys Ile Lys Asn Ile Leu
85 90 95
Trp Ser Asn Gly Asp Pro Leu Thr Ala Gln Asp Phe Val Ser Ser Trp
100 105 110
Lys Glu Ile Leu Lys Glu Asp Ala Ser Ser Val Tyr Leu Tyr Ala Phe
115 120 125
Leu Pro Ile Lys Asn Ala Arg Ala Ile Phe Asp Asp Thr Glu Ser Pro
130 135 140
Glu Asn Leu Gly Val Arg Ala Leu Asp Lys Arg His Leu Glu Ile Gln
145 150 155 160
Leu Glu Thr Pro Cys Ala His Phe Leu His Phe Leu Thr Leu Pro Ile
165 170 175
Phe Phe Pro Val His Glu Thr Leu Arg Asn Tyr Ser Thr Ser Phe Glu
180 185 190
Glu Met Pro Ile Thr Cys Gly Ala Phe Arg Pro Val Ser Leu Glu Lys
195 200 205
Gly Leu Arg Leu His Leu Glu Lys Asn Pro Met Tyr His Asn Lys Ser
210 215 220
Arg Val Lys Leu His Lys Ile Ile Val Gln Phe Ile Ser Asn Ala Asn
225 230 235 240
Thr Ala Ala Ile Leu Phe Lys His Lys Lys Leu Asp Trp Gln Gly Pro
245 250 255
Pro Trp Gly Glu Pro Ile Pro Pro Glu Ile Ser Ala Ser Leu His Gln
260 265 270
Asp Asp Gln Leu Phe Ser Leu Pro Gly Ala Ser Thr Thr Trp Leu Leu
275 280 285
<210> SEQ ID NO 21
<211> LENGTH: 480
<212> TYPE: DNA
<213> ORGANISM: Chlamydia pneumoniae
<400> SEQUENCE: 21
atgatgtttg ggcattttgc tggttacctt ggagcagatc ctgaagagcg aatgacttcc 60
aaaggaaaac gtgtgatcac tctgagactg ggagtgaaga ctcgagttgg aatgaaagat 120
gaaactgttt ggtgcaaatg caatatttgg cacaatcgct atgataagat gcttccttac 180
ttgaagaaag gctcaggagt cattgttgct ggcgatatct ctgtagagag ttacatgagc 240
aaagatggtt caccgcaatc ttctttagtg attagtgtag attctttgaa attcagtcct 300
ttcggtcgca atgaaggcag ccgttctcca tctttagaag acaatcatca gcaagtggga 360
tatgaatctg tatccgtagg gtttgaaggt gaagcactgg acgcagaagc tattaaagat 420
aaagatatgt atgctggtta tggtcaagaa cagcagtatg tctgtgaaga tgttcctttt 480
<210> SEQ ID NO 22
<211> LENGTH: 160
<212> TYPE: PRT
<213> ORGANISM: Chlamydia pneumoniae
<400> SEQUENCE: 22
Met Met Phe Gly His Phe Ala Gly Tyr Leu Gly Ala Asp Pro Glu Glu
1 5 10 15
Arg Met Thr Ser Lys Gly Lys Arg Val Ile Thr Leu Arg Leu Gly Val
20 25 30
Lys Thr Arg Val Gly Met Lys Asp Glu Thr Val Trp Cys Lys Cys Asn
35 40 45
Ile Trp His Asn Arg Tyr Asp Lys Met Leu Pro Tyr Leu Lys Lys Gly
50 55 60
Ser Gly Val Ile Val Ala Gly Asp Ile Ser Val Glu Ser Tyr Met Ser
65 70 75 80
Lys Asp Gly Ser Pro Gln Ser Ser Leu Val Ile Ser Val Asp Ser Leu
85 90 95
Lys Phe Ser Pro Phe Gly Arg Asn Glu Gly Ser Arg Ser Pro Ser Leu
100 105 110
Glu Asp Asn His Gln Gln Val Gly Tyr Glu Ser Val Ser Val Gly Phe
115 120 125
Glu Gly Glu Ala Leu Asp Ala Glu Ala Ile Lys Asp Lys Asp Met Tyr
130 135 140
Ala Gly Tyr Gly Gln Glu Gln Gln Tyr Val Cys Glu Asp Val Pro Phe
145 150 155 160
<210> SEQ ID NO 23
<211> LENGTH: 474
<212> TYPE: DNA
<213> ORGANISM: Chlamydia pneumoniae
<400> SEQUENCE: 23
atgacgcaag aaaagatcaa aatacatgtt tccaatgagc aaacatgtat tcctattcat 60
ttggtttctg tagagaagct ggttcttacg ctcttagagc acttaaaagt aacaactaat 120
gaaattttta tctacttcct agaagataaa gctcttgcag aactccatga taaggtattt 180
gctgatcctt ctctaacaga tacgatcact ctgcctattg atgctcccgg agatcccgct 240
tatcctcatg ttttaggaga agcattcatt agcccacagg ccgctcttag gtttttagag 300
aacacatccc caaaccaaga ggatatctac gaagaaatct cgagatacct cgtccactct 360
attctccata tgctcggata cgacgacacc tcatcagaag aaaagagaaa aatgagagtt 420
aaagaaaatc aaatcctgtg tatgttaaga aaaaaacatg ctttgctaac agct 474
<210> SEQ ID NO 24
<211> LENGTH: 158
<212> TYPE: PRT
<213> ORGANISM: Chlamydia pneumoniae
<400> SEQUENCE: 24
Met Thr Gln Glu Lys Ile Lys Ile His Val Ser Asn Glu Gln Thr Cys
1 5 10 15
Ile Pro Ile His Leu Val Ser Val Glu Lys Leu Val Leu Thr Leu Leu
20 25 30
Glu His Leu Lys Val Thr Thr Asn Glu Ile Phe Ile Tyr Phe Leu Glu
35 40 45
Asp Lys Ala Leu Ala Glu Leu His Asp Lys Val Phe Ala Asp Pro Ser
50 55 60
Leu Thr Asp Thr Ile Thr Leu Pro Ile Asp Ala Pro Gly Asp Pro Ala
65 70 75 80
Tyr Pro His Val Leu Gly Glu Ala Phe Ile Ser Pro Gln Ala Ala Leu
85 90 95
Arg Phe Leu Glu Asn Thr Ser Pro Asn Gln Glu Asp Ile Tyr Glu Glu
100 105 110
Ile Ser Arg Tyr Leu Val His Ser Ile Leu His Met Leu Gly Tyr Asp
115 120 125
Asp Thr Ser Ser Glu Glu Lys Arg Lys Met Arg Val Lys Glu Asn Gln
130 135 140
Ile Leu Cys Met Leu Arg Lys Lys His Ala Leu Leu Thr Ala
145 150 155
<210> SEQ ID NO 25
<211> LENGTH: 1452
<212> TYPE: DNA
<213> ORGANISM: Chlamydia pneumoniae
<400> SEQUENCE: 25
atgatcacac gcactaaaat tatttgcact atagggccag caacgaatag tccagagatg 60
ttagcaaaac ttctagatgc tgggatgaac gtagcaagat taaatttcag tcatgggagt 120
cacgaaactc atggacaggc tattggattt ctcaaggagt taagggagca gaagcgggtt 180
cctttagcaa ttatgctaga tactaagggg cctgaaattc gtttagggaa tattcctcag 240
ccaatttcgg tttctcaggg acaaaagctt cgtctggtaa gtagtgatat cgatgggagt 300
gctgaagggg gagtgtctct ctatcctaag gggatatttc cctttgttcc tgagggtgct 360
gatgttttaa tagatgatgg ctacattcat gctgttgttg tctcttcaga ggctgattct 420
ttagaattag agtttatgaa cagtggcctt ctcaagtctc ataaatcttt gagtatccga 480
ggtgttgatg ttgctcttcc ctttatgaca gagaaagata ttgcggatct taagtttggg 540
gtagagcaga atatggatgt ggttgctgca tcttttgtgc gctacggtga agatattgaa 600
actatgcgca agtgtttagc agacttaggc aatcctaaga tgcccatcat tgcaaaaata 660
gaaaatcgtt taggggtaga aaatttctct aagattgcca agcttgcgga tggaattatg 720
attgctagag gagatttagg aatcgagctt tctgtcgttg aagtcccaaa tttgcaaaag 780
atgatggcta aggtttctag agaaacaggt cacttctgtg tgactgcaac gcagatgcta 840
gaatctatga ttcgcaatgt cttacctaca cgagctgaag tctctgatat tgccaatgca 900
atttatgatg gttcttcagc agtgatgttg tcaggggaaa ctgcatctgg agcccatccc 960
gtggctgccg tgaaaatcat gcgttctgtg attttagaaa cagaaaagaa tctctcccat 1020
gattcattct taaaattaga cgatagcaat agcgctcttc aggtgtcccc ctatctctca 1080
gccattggat tggcaggcat tcagattgca gaaagggcag acgccaaagc tcttattgtt 1140
tatacagaat caggaagttc tccgatgttt ctctctaaat atcgtccgaa attccctatc 1200
attgccgtga ctccaagcac ttctgtttac tatcgcctag ctttggaatg gggggtctat 1260
cctatgctta cccaggaaag tgatcgcgct gtatggagac atcaggcctg tatttatggc 1320
atagaacagg gcattctctc taattatgat cggattcttg tgcttagcag aggagcctgt 1380
atggaagaaa caaataatct taccctgaca atagtgaatg atattttgac tgggtcggaa 1440
tttcctgaaa cc 1452
<210> SEQ ID NO 26
<211> LENGTH: 484
<212> TYPE: PRT
<213> ORGANISM: Chlamydia pneumoniae
<400> SEQUENCE: 26
Met Ile Thr Arg Thr Lys Ile Ile Cys Thr Ile Gly Pro Ala Thr Asn
1 5 10 15
Ser Pro Glu Met Leu Ala Lys Leu Leu Asp Ala Gly Met Asn Val Ala
20 25 30
Arg Leu Asn Phe Ser His Gly Ser His Glu Thr His Gly Gln Ala Ile
35 40 45
Gly Phe Leu Lys Glu Leu Arg Glu Gln Lys Arg Val Pro Leu Ala Ile
50 55 60
Met Leu Asp Thr Lys Gly Pro Glu Ile Arg Leu Gly Asn Ile Pro Gln
65 70 75 80
Pro Ile Ser Val Ser Gln Gly Gln Lys Leu Arg Leu Val Ser Ser Asp
85 90 95
Ile Asp Gly Ser Ala Glu Gly Gly Val Ser Leu Tyr Pro Lys Gly Ile
100 105 110
Phe Pro Phe Val Pro Glu Gly Ala Asp Val Leu Ile Asp Asp Gly Tyr
115 120 125
Ile His Ala Val Val Val Ser Ser Glu Ala Asp Ser Leu Glu Leu Glu
130 135 140
Phe Met Asn Ser Gly Leu Leu Lys Ser His Lys Ser Leu Ser Ile Arg
145 150 155 160
Gly Val Asp Val Ala Leu Pro Phe Met Thr Glu Lys Asp Ile Ala Asp
165 170 175
Leu Lys Phe Gly Val Glu Gln Asn Met Asp Val Val Ala Ala Ser Phe
180 185 190
Val Arg Tyr Gly Glu Asp Ile Glu Thr Met Arg Lys Cys Leu Ala Asp
195 200 205
Leu Gly Asn Pro Lys Met Pro Ile Ile Ala Lys Ile Glu Asn Arg Leu
210 215 220
Gly Val Glu Asn Phe Ser Lys Ile Ala Lys Leu Ala Asp Gly Ile Met
225 230 235 240
Ile Ala Arg Gly Asp Leu Gly Ile Glu Leu Ser Val Val Glu Val Pro
245 250 255
Asn Leu Gln Lys Met Met Ala Lys Val Ser Arg Glu Thr Gly His Phe
260 265 270
Cys Val Thr Ala Thr Gln Met Leu Glu Ser Met Ile Arg Asn Val Leu
275 280 285
Pro Thr Arg Ala Glu Val Ser Asp Ile Ala Asn Ala Ile Tyr Asp Gly
290 295 300
Ser Ser Ala Val Met Leu Ser Gly Glu Thr Ala Ser Gly Ala His Pro
305 310 315 320
Val Ala Ala Val Lys Ile Met Arg Ser Val Ile Leu Glu Thr Glu Lys
325 330 335
Asn Leu Ser His Asp Ser Phe Leu Lys Leu Asp Asp Ser Asn Ser Ala
340 345 350
Leu Gln Val Ser Pro Tyr Leu Ser Ala Ile Gly Leu Ala Gly Ile Gln
355 360 365
Ile Ala Glu Arg Ala Asp Ala Lys Ala Leu Ile Val Tyr Thr Glu Ser
370 375 380
Gly Ser Ser Pro Met Phe Leu Ser Lys Tyr Arg Pro Lys Phe Pro Ile
385 390 395 400
Ile Ala Val Thr Pro Ser Thr Ser Val Tyr Tyr Arg Leu Ala Leu Glu
405 410 415
Trp Gly Val Tyr Pro Met Leu Thr Gln Glu Ser Asp Arg Ala Val Trp
420 425 430
Arg His Gln Ala Cys Ile Tyr Gly Ile Glu Gln Gly Ile Leu Ser Asn
435 440 445
Tyr Asp Arg Ile Leu Val Leu Ser Arg Gly Ala Cys Met Glu Glu Thr
450 455 460
Asn Asn Leu Thr Leu Thr Ile Val Asn Asp Ile Leu Thr Gly Ser Glu
465 470 475 480
Phe Pro Glu Thr
<210> SEQ ID NO 27
<211> LENGTH: 1992
<212> TYPE: DNA
<213> ORGANISM: Chlamydia pneumoniae
<400> SEQUENCE: 27
atggaaaaag tttcttctta tccctcagtt cctttacctc ttggggcttc taaaatttcc 60
ccaaaccgct atcgatttgc tttatatgct tcacaagcta ccgaagtcat ccttgcttta 120
acagacgaaa attcagaagt catagaagtc cctctttacc ccgatacaca ccgcacgggt 180
gcgatttggc atatagagat cgagggtatt tctgatcaat cgtcttatgc atttcgtgtt 240
catgggccta aaaagcatgg aatgcaatac tcttttaaag aatatcttgc agatccctat 300
gcgaagaata ttcattcccc acagagtttt ggttcgcgaa agaaacaggg ggattatgca 360
ttttgttatt taaaggaaga accatttcct tgggatggtg atcagcctct gcatttgccg 420
aaagaagaga tgatcatcta tgagatgcat gtacgttcct tcacgcaatc ttcttcatct 480
agggttcatg ctccgggaac cttcctagga atcattgaaa agatcgacca tctgcataag 540
ctgggaatca acgctgttga actcttacct atctttgagt tcgatgagac tgcgcatcct 600
tttagaaatt cgaaattccc ttatctgtgc aattattggg gttatgctcc cctaaatttc 660
ttttctcctt gccgacgtta tgcttatgcc tctgatcctt gcgctccaag tagagagttt 720
aaaactttag taaagacctt gcatcaagaa ggtattgagg tcattcttga tgttgttttt 780
aatcatacgg gcttgcaagg gacgacctgc tctttgcctt ggatagacac tccgagctat 840
tatattttag atgcacaagg tcactttaca aattattcag gctgtggaaa cactctcaat 900
acaaaccgcg cccccacgac ccaatggatt ctcgacatct tacgttattg ggtagaagaa 960
atgcatgtcg atgggttccg atttgatctt gcttctgtct tttctcgtgg tccttcggga 1020
tctcccctac aattcgctcc tgttttagag gcgatttctt ttgatccttt acttgcgagc 1080
acaaagatta tagctgagcc ttgggatgct ggcggtttgt atcaggtggg ctatttcccc 1140
acactgtctc caagatggag tgaatggaac ggcccgtatc gtgataacgt gaaagcattt 1200
cttaatgggg atcaaaatct cataggaacc tttgcttcta gaatttcagg atctcaagac 1260
atctatcctc acggctcgcc tacaaattcg attaactatg tcagttgcca tgatggtttt 1320
acgttatgtg acactgtgac ttataaccac aaacataatg aggctaacgg agaggataat 1380
cgtgacggca cagatgcgaa ctacagctac aatttcggaa cggaagggaa aacagaagac 1440
cctggcattc ttgaagttcg tgaaagacag ttacgaaatt ttttccttac tttgatggtc 1500
tcgcaaggca ttccgatgat tcaatcagga gatgagtatg cccataccgc ggaaggcaat 1560
aacaaccgtt gggctttgga ttcgaatgcg aattacttcc tttgggatca gcttaccgca 1620
aagcctacac tgatgcactt tctctgtgat ctcattgcgt ttcgaaaaaa atataaaaca 1680
ctttttaatc gaggctttct ttccaataag gaaatcagtt gggtagatgc tatgggaaat 1740
cccatgacat ggcgccctgg aaatttctta gcatttaaaa taaaatcgcc aaaagcgcat 1800
gtatatgttg cttttcacgt gggagctcaa gaccaacttg cgaccttacc taaagcctcc 1860
agcaactttc ttccttatca aatagttgcc gagagtcagc aagggtttgt ccctcaaaat 1920
gtagcaacgc cgacagtgtc gctacagccc cataccacgc taattgcgat cagccatgcg 1980
aaagaggtta cc 1992
<210> SEQ ID NO 28
<211> LENGTH: 664
<212> TYPE: PRT
<213> ORGANISM: Chlamydia pneumoniae
<400> SEQUENCE: 28
Met Glu Lys Val Ser Ser Tyr Pro Ser Val Pro Leu Pro Leu Gly Ala
1 5 10 15
Ser Lys Ile Ser Pro Asn Arg Tyr Arg Phe Ala Leu Tyr Ala Ser Gln
20 25 30
Ala Thr Glu Val Ile Leu Ala Leu Thr Asp Glu Asn Ser Glu Val Ile
35 40 45
Glu Val Pro Leu Tyr Pro Asp Thr His Arg Thr Gly Ala Ile Trp His
50 55 60
Ile Glu Ile Glu Gly Ile Ser Asp Gln Ser Ser Tyr Ala Phe Arg Val
65 70 75 80
His Gly Pro Lys Lys His Gly Met Gln Tyr Ser Phe Lys Glu Tyr Leu
85 90 95
Ala Asp Pro Tyr Ala Lys Asn Ile His Ser Pro Gln Ser Phe Gly Ser
100 105 110
Arg Lys Lys Gln Gly Asp Tyr Ala Phe Cys Tyr Leu Lys Glu Glu Pro
115 120 125
Phe Pro Trp Asp Gly Asp Gln Pro Leu His Leu Pro Lys Glu Glu Met
130 135 140
Ile Ile Tyr Glu Met His Val Arg Ser Phe Thr Gln Ser Ser Ser Ser
145 150 155 160
Arg Val His Ala Pro Gly Thr Phe Leu Gly Ile Ile Glu Lys Ile Asp
165 170 175
His Leu His Lys Leu Gly Ile Asn Ala Val Glu Leu Leu Pro Ile Phe
180 185 190
Glu Phe Asp Glu Thr Ala His Pro Phe Arg Asn Ser Lys Phe Pro Tyr
195 200 205
Leu Cys Asn Tyr Trp Gly Tyr Ala Pro Leu Asn Phe Phe Ser Pro Cys
210 215 220
Arg Arg Tyr Ala Tyr Ala Ser Asp Pro Cys Ala Pro Ser Arg Glu Phe
225 230 235 240
Lys Thr Leu Val Lys Thr Leu His Gln Glu Gly Ile Glu Val Ile Leu
245 250 255
Asp Val Val Phe Asn His Thr Gly Leu Gln Gly Thr Thr Cys Ser Leu
260 265 270
Pro Trp Ile Asp Thr Pro Ser Tyr Tyr Ile Leu Asp Ala Gln Gly His
275 280 285
Phe Thr Asn Tyr Ser Gly Cys Gly Asn Thr Leu Asn Thr Asn Arg Ala
290 295 300
Pro Thr Thr Gln Trp Ile Leu Asp Ile Leu Arg Tyr Trp Val Glu Glu
305 310 315 320
Met His Val Asp Gly Phe Arg Phe Asp Leu Ala Ser Val Phe Ser Arg
325 330 335
Gly Pro Ser Gly Ser Pro Leu Gln Phe Ala Pro Val Leu Glu Ala Ile
340 345 350
Ser Phe Asp Pro Leu Leu Ala Ser Thr Lys Ile Ile Ala Glu Pro Trp
355 360 365
Asp Ala Gly Gly Leu Tyr Gln Val Gly Tyr Phe Pro Thr Leu Ser Pro
370 375 380
Arg Trp Ser Glu Trp Asn Gly Pro Tyr Arg Asp Asn Val Lys Ala Phe
385 390 395 400
Leu Asn Gly Asp Gln Asn Leu Ile Gly Thr Phe Ala Ser Arg Ile Ser
405 410 415
Gly Ser Gln Asp Ile Tyr Pro His Gly Ser Pro Thr Asn Ser Ile Asn
420 425 430
Tyr Val Ser Cys His Asp Gly Phe Thr Leu Cys Asp Thr Val Thr Tyr
435 440 445
Asn His Lys His Asn Glu Ala Asn Gly Glu Asp Asn Arg Asp Gly Thr
450 455 460
Asp Ala Asn Tyr Ser Tyr Asn Phe Gly Thr Glu Gly Lys Thr Glu Asp
465 470 475 480
Pro Gly Ile Leu Glu Val Arg Glu Arg Gln Leu Arg Asn Phe Phe Leu
485 490 495
Thr Leu Met Val Ser Gln Gly Ile Pro Met Ile Gln Ser Gly Asp Glu
500 505 510
Tyr Ala His Thr Ala Glu Gly Asn Asn Asn Arg Trp Ala Leu Asp Ser
515 520 525
Asn Ala Asn Tyr Phe Leu Trp Asp Gln Leu Thr Ala Lys Pro Thr Leu
530 535 540
Met His Phe Leu Cys Asp Leu Ile Ala Phe Arg Lys Lys Tyr Lys Thr
545 550 555 560
Leu Phe Asn Arg Gly Phe Leu Ser Asn Lys Glu Ile Ser Trp Val Asp
565 570 575
Ala Met Gly Asn Pro Met Thr Trp Arg Pro Gly Asn Phe Leu Ala Phe
580 585 590
Lys Ile Lys Ser Pro Lys Ala His Val Tyr Val Ala Phe His Val Gly
595 600 605
Ala Gln Asp Gln Leu Ala Thr Leu Pro Lys Ala Ser Ser Asn Phe Leu
610 615 620
Pro Tyr Gln Ile Val Ala Glu Ser Gln Gln Gly Phe Val Pro Gln Asn
625 630 635 640
Val Ala Thr Pro Thr Val Ser Leu Gln Pro His Thr Thr Leu Ile Ala
645 650 655
Ile Ser His Ala Lys Glu Val Thr
660
<210> SEQ ID NO 29
<211> LENGTH: 993
<212> TYPE: DNA
<213> ORGANISM: Chlamydia pneumoniae
<400> SEQUENCE: 29
ttttctcgtg gtccttcggg atctccccta caattcgctc ctgttttaga ggcgatttct 60
tttgatcctt tacttgcgag cacaaagatt atagctgagc cttgggatgc tggcggtttg 120
tatcaggtgg gctatttccc cacactgtct ccaagatgga gtgaatggaa cggcccgtat 180
cgtgataacg tgaaagcatt tcttaatggg gatcaaaatc tcataggaac ctttgcttct 240
agaatttcag gatctcaaga catctatcct cacggctcgc ctacaaattc gattaactat 300
gtcagttgcc atgatggttt tacgttatgt gacactgtga cttataacca caaacataat 360
gaggctaacg gagaggataa tcgtgacggc acagatgcga actacagcta caatttcgga 420
acggaaggga aaacagaaga ccctggcatt cttgaagttc gtgaaagaca gttacgaaat 480
tttttcctta ctttgatggt ctcgcaaggc attccgatga ttcaatcagg agatgagtat 540
gcccataccg cggaaggcaa taacaaccgt tgggctttgg attcgaatgc gaattacttc 600
ctttgggatc agcttaccgc aaagcctaca ctgatgcact ttctctgtga tctcattgcg 660
tttcgaaaaa aatataaaac actttttaat cgaggctttc tttccaataa ggaaatcagt 720
tgggtagatg ctatgggaaa tcccatgaca tggcgccctg gaaatttctt agcatttaaa 780
ataaaatcgc caaaagcgca tgtatatgtt gcttttcacg tgggagctca agaccaactt 840
gcgaccttac ctaaagcctc cagcaacttt cttccttatc aaatagttgc cgagagtcag 900
caagggtttg tccctcaaaa tgtagcaacg ccgacagtgt cgctacagcc ccataccacg 960
ctaattgcga tcagccatgc gaaagaggtt acc 993
<210> SEQ ID NO 30
<211> LENGTH: 331
<212> TYPE: PRT
<213> ORGANISM: Chlamydia pneumoniae
<400> SEQUENCE: 30
Phe Ser Arg Gly Pro Ser Gly Ser Pro Leu Gln Phe Ala Pro Val Leu
1 5 10 15
Glu Ala Ile Ser Phe Asp Pro Leu Leu Ala Ser Thr Lys Ile Ile Ala
20 25 30
Glu Pro Trp Asp Ala Gly Gly Leu Tyr Gln Val Gly Tyr Phe Pro Thr
35 40 45
Leu Ser Pro Arg Trp Ser Glu Trp Asn Gly Pro Tyr Arg Asp Asn Val
50 55 60
Lys Ala Phe Leu Asn Gly Asp Gln Asn Leu Ile Gly Thr Phe Ala Ser
65 70 75 80
Arg Ile Ser Gly Ser Gln Asp Ile Tyr Pro His Gly Ser Pro Thr Asn
85 90 95
Ser Ile Asn Tyr Val Ser Cys His Asp Gly Phe Thr Leu Cys Asp Thr
100 105 110
Val Thr Tyr Asn His Lys His Asn Glu Ala Asn Gly Glu Asp Asn Arg
115 120 125
Asp Gly Thr Asp Ala Asn Tyr Ser Tyr Asn Phe Gly Thr Glu Gly Lys
130 135 140
Thr Glu Asp Pro Gly Ile Leu Glu Val Arg Glu Arg Gln Leu Arg Asn
145 150 155 160
Phe Phe Leu Thr Leu Met Val Ser Gln Gly Ile Pro Met Ile Gln Ser
165 170 175
Gly Asp Glu Tyr Ala His Thr Ala Glu Gly Asn Asn Asn Arg Trp Ala
180 185 190
Leu Asp Ser Asn Ala Asn Tyr Phe Leu Trp Asp Gln Leu Thr Ala Lys
195 200 205
Pro Thr Leu Met His Phe Leu Cys Asp Leu Ile Ala Phe Arg Lys Lys
210 215 220
Tyr Lys Thr Leu Phe Asn Arg Gly Phe Leu Ser Asn Lys Glu Ile Ser
225 230 235 240
Trp Val Asp Ala Met Gly Asn Pro Met Thr Trp Arg Pro Gly Asn Phe
245 250 255
Leu Ala Phe Lys Ile Lys Ser Pro Lys Ala His Val Tyr Val Ala Phe
260 265 270
His Val Gly Ala Gln Asp Gln Leu Ala Thr Leu Pro Lys Ala Ser Ser
275 280 285
Asn Phe Leu Pro Tyr Gln Ile Val Ala Glu Ser Gln Gln Gly Phe Val
290 295 300
Pro Gln Asn Val Ala Thr Pro Thr Val Ser Leu Gln Pro His Thr Thr
305 310 315 320
Leu Ile Ala Ile Ser His Ala Lys Glu Val Thr
325 330
<210> SEQ ID NO 31
<211> LENGTH: 2109
<212> TYPE: DNA
<213> ORGANISM: Chlamydia pneumoniae
<400> SEQUENCE: 31
tggagtaacc ccaacctacg tcttatgaaa cgttgcttct tatttctagc ttcctttgtt 60
cttatgggtt cctcagctga tgctttgact catcaagagg ctgtgaaaaa gaaaaactcc 120
tatcttagtc actttaagag tgtttctggg attgtgacca tcgaagatgg ggtattgaat 180
atccataaca acctgcggat acaagccaat aaagtgtatg tagaaaatac tgtgggtcaa 240
agcctgaagc ttgtcgcaca tggcaatgtt atggtgaact atagggcaaa aaccctagtt 300
tgtgattacc tagagtatta cgaagataca gactcttgtc ttcttactaa tggaagattc 360
gcgatgtatc cttggtttct aggggggtct atgatcactc taaccccaga aaccatagtc 420
attcggaagg gatatatctc tacctccgag ggtcccaaaa aagacctgtg cctctccgga 480
gattacctgg aatattcttc agatagtctt ctttctatag ggaagacaac attaagggtg 540
tgtcgcattc cgatactttt cttacctcca ttttctatca tgcctatgga gatccctaag 600
cctccgataa actttcgagg aggaacagga ggatttctgg gatcctattt ggggatgagc 660
tactcgccga tttctaggaa gcatttctcc tcgacatttt tcttggatag ctttttcaag 720
catggcgtcg gcatgggatt caacctccat tgttctcaga agcaggttcc tgagaatgtc 780
ttcaatatga aaagctatta tgcccaccgc cttgctatcg atatggcaga agctcatgat 840
cgctatcgcc tacacggaga tttctgcttc acgcataagc atgtaaattt ttctggagaa 900
taccatctca gcgatagttg ggaaactgtt gctgacattt tccccaacaa cttcatgttg 960
aaaaatacag gccccacacg tgtcgattgc acttggaatg acaactattt tgaagggtat 1020
ctcacctctt ctgttaaggt aaactctttc caaaatgcca accaagagct cccttattta 1080
acattaaggc agtacccgat ttctatttat aatacgggag tgtaccttga aaacatcgta 1140
gaatgtgggt atttaaactt tgcttttagc gatcatatcg ttggcgagaa tttctcttca 1200
ctacgtcttg ctgcgcgccc taagctccat aaaactgtgc ctctacctat aggaacgctc 1260
tcctccaccc tagggagttc tctgatttac tatagcgatg ttcctgagat ctcctcgcgc 1320
catagtcagc tttccgcgaa gctacaactt gattatcgct ttctattaca taagtcctac 1380
attcaaagac gccatattat agagccgttc gttaccttca ttacagagac tcgtcctcta 1440
gctaagaatg aagatcatta tatcttttct attcaagatg cctttcactc cttaaacctt 1500
ctgaaagcgg gtatagatac ctcggtactg agtaagacta accctcgatt cccgagaatc 1560
catgcgaagc tgtggactac ccacatcttg agcaatacag aaagcaaacc cacgtttccc 1620
aaaactgcat gcgagctatc tctacctttt ggaaagaaaa atacagtctc cttagatgct 1680
gaatggattt ggaaaaagca ctgttgggat cacatgaaca tacgttggga gtggatcgga 1740
aatgacaatg tggctatgac tctagaatcc ctgcatagaa gcaaatacag cctgattaag 1800
tgtgacaggg agaacttcat tttagatgtc agccgtccca ttgaccagct tttagactcc 1860
cctctctctg atcataggaa tctcatttta gggaaattat ttgtacgacc tcatccctgt 1920
tggaattacc gcttatcctt acgctatggc tggcatcgcc aggacactcc gaactaccta 1980
gaataccaga tgattctagg gacgaagatc ttcgaacatt ggcagctcta tggggtgtat 2040
gaacgccgag aagcagatag tcgatttttc ttcttcttaa agctcgacaa acctaaaaaa 2100
cctcccttc 2109
<210> SEQ ID NO 32
<211> LENGTH: 703
<212> TYPE: PRT
<213> ORGANISM: Chlamydia pneumoniae
<400> SEQUENCE: 32
Trp Ser Asn Pro Asn Leu Arg Leu Met Lys Arg Cys Phe Leu Phe Leu
1 5 10 15
Ala Ser Phe Val Leu Met Gly Ser Ser Ala Asp Ala Leu Thr His Gln
20 25 30
Glu Ala Val Lys Lys Lys Asn Ser Tyr Leu Ser His Phe Lys Ser Val
35 40 45
Ser Gly Ile Val Thr Ile Glu Asp Gly Val Leu Asn Ile His Asn Asn
50 55 60
Leu Arg Ile Gln Ala Asn Lys Val Tyr Val Glu Asn Thr Val Gly Gln
65 70 75 80
Ser Leu Lys Leu Val Ala His Gly Asn Val Met Val Asn Tyr Arg Ala
85 90 95
Lys Thr Leu Val Cys Asp Tyr Leu Glu Tyr Tyr Glu Asp Thr Asp Ser
100 105 110
Cys Leu Leu Thr Asn Gly Arg Phe Ala Met Tyr Pro Trp Phe Leu Gly
115 120 125
Gly Ser Met Ile Thr Leu Thr Pro Glu Thr Ile Val Ile Arg Lys Gly
130 135 140
Tyr Ile Ser Thr Ser Glu Gly Pro Lys Lys Asp Leu Cys Leu Ser Gly
145 150 155 160
Asp Tyr Leu Glu Tyr Ser Ser Asp Ser Leu Leu Ser Ile Gly Lys Thr
165 170 175
Thr Leu Arg Val Cys Arg Ile Pro Ile Leu Phe Leu Pro Pro Phe Ser
180 185 190
Ile Met Pro Met Glu Ile Pro Lys Pro Pro Ile Asn Phe Arg Gly Gly
195 200 205
Thr Gly Gly Phe Leu Gly Ser Tyr Leu Gly Met Ser Tyr Ser Pro Ile
210 215 220
Ser Arg Lys His Phe Ser Ser Thr Phe Phe Leu Asp Ser Phe Phe Lys
225 230 235 240
His Gly Val Gly Met Gly Phe Asn Leu His Cys Ser Gln Lys Gln Val
245 250 255
Pro Glu Asn Val Phe Asn Met Lys Ser Tyr Tyr Ala His Arg Leu Ala
260 265 270
Ile Asp Met Ala Glu Ala His Asp Arg Tyr Arg Leu His Gly Asp Phe
275 280 285
Cys Phe Thr His Lys His Val Asn Phe Ser Gly Glu Tyr His Leu Ser
290 295 300
Asp Ser Trp Glu Thr Val Ala Asp Ile Phe Pro Asn Asn Phe Met Leu
305 310 315 320
Lys Asn Thr Gly Pro Thr Arg Val Asp Cys Thr Trp Asn Asp Asn Tyr
325 330 335
Phe Glu Gly Tyr Leu Thr Ser Ser Val Lys Val Asn Ser Phe Gln Asn
340 345 350
Ala Asn Gln Glu Leu Pro Tyr Leu Thr Leu Arg Gln Tyr Pro Ile Ser
355 360 365
Ile Tyr Asn Thr Gly Val Tyr Leu Glu Asn Ile Val Glu Cys Gly Tyr
370 375 380
Leu Asn Phe Ala Phe Ser Asp His Ile Val Gly Glu Asn Phe Ser Ser
385 390 395 400
Leu Arg Leu Ala Ala Arg Pro Lys Leu His Lys Thr Val Pro Leu Pro
405 410 415
Ile Gly Thr Leu Ser Ser Thr Leu Gly Ser Ser Leu Ile Tyr Tyr Ser
420 425 430
Asp Val Pro Glu Ile Ser Ser Arg His Ser Gln Leu Ser Ala Lys Leu
435 440 445
Gln Leu Asp Tyr Arg Phe Leu Leu His Lys Ser Tyr Ile Gln Arg Arg
450 455 460
His Ile Ile Glu Pro Phe Val Thr Phe Ile Thr Glu Thr Arg Pro Leu
465 470 475 480
Ala Lys Asn Glu Asp His Tyr Ile Phe Ser Ile Gln Asp Ala Phe His
485 490 495
Ser Leu Asn Leu Leu Lys Ala Gly Ile Asp Thr Ser Val Leu Ser Lys
500 505 510
Thr Asn Pro Arg Phe Pro Arg Ile His Ala Lys Leu Trp Thr Thr His
515 520 525
Ile Leu Ser Asn Thr Glu Ser Lys Pro Thr Phe Pro Lys Thr Ala Cys
530 535 540
Glu Leu Ser Leu Pro Phe Gly Lys Lys Asn Thr Val Ser Leu Asp Ala
545 550 555 560
Glu Trp Ile Trp Lys Lys His Cys Trp Asp His Met Asn Ile Arg Trp
565 570 575
Glu Trp Ile Gly Asn Asp Asn Val Ala Met Thr Leu Glu Ser Leu His
580 585 590
Arg Ser Lys Tyr Ser Leu Ile Lys Cys Asp Arg Glu Asn Phe Ile Leu
595 600 605
Asp Val Ser Arg Pro Ile Asp Gln Leu Leu Asp Ser Pro Leu Ser Asp
610 615 620
His Arg Asn Leu Ile Leu Gly Lys Leu Phe Val Arg Pro His Pro Cys
625 630 635 640
Trp Asn Tyr Arg Leu Ser Leu Arg Tyr Gly Trp His Arg Gln Asp Thr
645 650 655
Pro Asn Tyr Leu Glu Tyr Gln Met Ile Leu Gly Thr Lys Ile Phe Glu
660 665 670
His Trp Gln Leu Tyr Gly Val Tyr Glu Arg Arg Glu Ala Asp Ser Arg
675 680 685
Phe Phe Phe Phe Leu Lys Leu Asp Lys Pro Lys Lys Pro Pro Phe
690 695 700
<210> SEQ ID NO 33
<211> LENGTH: 1092
<212> TYPE: DNA
<213> ORGANISM: Chlamydia pneumoniae
<400> SEQUENCE: 33
tatctcacct cttctgttaa ggtaaactct ttccaaaatg ccaaccaaga gctcccttat 60
ttaacattaa ggcagtaccc gatttctatt tataatacgg gagtgtacct tgaaaacatc 120
gtagaatgtg ggtatttaaa ctttgctttt agcgatcata tcgttggcga gaatttctct 180
tcactacgtc ttgctgcgcg ccctaagctc cataaaactg tgcctctacc tataggaacg 240
ctctcctcca ccctagggag ttctctgatt tactatagcg atgttcctga gatctcctcg 300
cgccatagtc agctttccgc gaagctacaa cttgattatc gctttctatt acataagtcc 360
tacattcaaa gacgccatat tatagagccg ttcgttacct tcattacaga gactcgtcct 420
ctagctaaga atgaagatca ttatatcttt tctattcaag atgcctttca ctccttaaac 480
cttctgaaag cgggtataga tacctcggta ctgagtaaga ctaaccctcg attcccgaga 540
atccatgcga agctgtggac tacccacatc ttgagcaata cagaaagcaa acccacgttt 600
cccaaaactg catgcgagct atctctacct tttggaaaga aaaatacagt ctccttagat 660
gctgaatgga tttggaaaaa gcactgttgg gatcacatga acatacgttg ggagtggatc 720
ggaaatgaca atgtggctat gactctagaa tccctgcata gaagcaaata cagcctgatt 780
aagtgtgaca gggagaactt cattttagat gtcagccgtc ccattgacca gcttttagac 840
tcccctctct ctgatcatag gaatctcatt ttagggaaat tatttgtacg acctcatccc 900
tgttggaatt accgcttatc cttacgctat ggctggcatc gccaggacac tccgaactac 960
ctagaatacc agatgattct agggacgaag atcttcgaac attggcagct ctatggggtg 1020
tatgaacgcc gagaagcaga tagtcgattt ttcttcttct taaagctcga caaacctaaa 1080
aaacctccct tc 1092
<210> SEQ ID NO 34
<211> LENGTH: 364
<212> TYPE: PRT
<213> ORGANISM: Chlamydia pneumoniae
<400> SEQUENCE: 34
Tyr Leu Thr Ser Ser Val Lys Val Asn Ser Phe Gln Asn Ala Asn Gln
1 5 10 15
Glu Leu Pro Tyr Leu Thr Leu Arg Gln Tyr Pro Ile Ser Ile Tyr Asn
20 25 30
Thr Gly Val Tyr Leu Glu Asn Ile Val Glu Cys Gly Tyr Leu Asn Phe
35 40 45
Ala Phe Ser Asp His Ile Val Gly Glu Asn Phe Ser Ser Leu Arg Leu
50 55 60
Ala Ala Arg Pro Lys Leu His Lys Thr Val Pro Leu Pro Ile Gly Thr
65 70 75 80
Leu Ser Ser Thr Leu Gly Ser Ser Leu Ile Tyr Tyr Ser Asp Val Pro
85 90 95
Glu Ile Ser Ser Arg His Ser Gln Leu Ser Ala Lys Leu Gln Leu Asp
100 105 110
Tyr Arg Phe Leu Leu His Lys Ser Tyr Ile Gln Arg Arg His Ile Ile
115 120 125
Glu Pro Phe Val Thr Phe Ile Thr Glu Thr Arg Pro Leu Ala Lys Asn
130 135 140
Glu Asp His Tyr Ile Phe Ser Ile Gln Asp Ala Phe His Ser Leu Asn
145 150 155 160
Leu Leu Lys Ala Gly Ile Asp Thr Ser Val Leu Ser Lys Thr Asn Pro
165 170 175
Arg Phe Pro Arg Ile His Ala Lys Leu Trp Thr Thr His Ile Leu Ser
180 185 190
Asn Thr Glu Ser Lys Pro Thr Phe Pro Lys Thr Ala Cys Glu Leu Ser
195 200 205
Leu Pro Phe Gly Lys Lys Asn Thr Val Ser Leu Asp Ala Glu Trp Ile
210 215 220
Trp Lys Lys His Cys Trp Asp His Met Asn Ile Arg Trp Glu Trp Ile
225 230 235 240
Gly Asn Asp Asn Val Ala Met Thr Leu Glu Ser Leu His Arg Ser Lys
245 250 255
Tyr Ser Leu Ile Lys Cys Asp Arg Glu Asn Phe Ile Leu Asp Val Ser
260 265 270
Arg Pro Ile Asp Gln Leu Leu Asp Ser Pro Leu Ser Asp His Arg Asn
275 280 285
Leu Ile Leu Gly Lys Leu Phe Val Arg Pro His Pro Cys Trp Asn Tyr
290 295 300
Arg Leu Ser Leu Arg Tyr Gly Trp His Arg Gln Asp Thr Pro Asn Tyr
305 310 315 320
Leu Glu Tyr Gln Met Ile Leu Gly Thr Lys Ile Phe Glu His Trp Gln
325 330 335
Leu Tyr Gly Val Tyr Glu Arg Arg Glu Ala Asp Ser Arg Phe Phe Phe
340 345 350
Phe Leu Lys Leu Asp Lys Pro Lys Lys Pro Pro Phe
355 360
<210> SEQ ID NO 35
<211> LENGTH: 1182
<212> TYPE: DNA
<213> ORGANISM: Chlamydia pneumoniae
<400> SEQUENCE: 35
atgaatccat ccaggggaga gaacatggcg attaaaaata tacttgttgt tgatgacgag 60
cccctactca gagatttcct ctcggaactt cttacctcac agggattcat cccagacact 120
gctgaaaact taagaaatgc tctccaaatg atccgaagtc gagactatga ccttgtcatc 180
tcagacatga gtatgcctga cggctctggt cttgatttaa tcaaaattat aaagcaaagc 240
tccccccaca cgcccgtcct tgtagtcact gcttacggaa gcatagagaa cgccgtagag 300
gctatgcacc aaggggcatt caactactta acaaaacctt tttcttctga agcacttttt 360
gcctttatct ctaaagctga agaacttaag aacctagtcc atgagaatct ctttctacat 420
tctcagacaa caccagattc acaccctctg attgcagaaa gcaaggctat gaaagatctt 480
cttgccatag caaaaaaagc agcttcaagc tcagcaaata tattcattca cggagaatcg 540
ggatgcggaa aggaagtcct ctcctttttt atccaccaca actctcctcg agccaaccac 600
ccctatatta aagttaactg cgcagcaatt cctgaaactc tcttagaatc agaacttttt 660
ggccatgaaa agggagcatt tacaggagca actacaaaga aggcaggacg ttttgaactt 720
gcccataaag gaaccctctt attagatgaa atcaccgaag tcccagtaaa ccttcaagca 780
aaactcctga gagctatcca agaaaaagaa atcgaacacc ttggaggaac caagaccctc 840
tccgtagatg ttcgcatctt agcgacctca aaccgaaagc ttaaagaagc tatcgatgat 900
aaaagcttcc gacaagatct gtattaccgg ttgaatgtca tccctctaca cctcccccct 960
ctaagagacc gacaggacga catcctccct ctggcgaact acttcctaaa taagttctgc 1020
cgcatgaaca atactcctct gaaaaccctc tctcctaaag ctcaagagct cctccttaac 1080
tacccctggc caggcaatat tcgagagctc tccaatgttc tggaacgtgt ggttatccta 1140
gagaacacct ccctactcac cgaagacatg ctcgctttag ct 1182
<210> SEQ ID NO 36
<211> LENGTH: 394
<212> TYPE: PRT
<213> ORGANISM: Chlamydia pneumoniae
<400> SEQUENCE: 36
Met Asn Pro Ser Arg Gly Glu Asn Met Ala Ile Lys Asn Ile Leu Val
1 5 10 15
Val Asp Asp Glu Pro Leu Leu Arg Asp Phe Leu Ser Glu Leu Leu Thr
20 25 30
Ser Gln Gly Phe Ile Pro Asp Thr Ala Glu Asn Leu Arg Asn Ala Leu
35 40 45
Gln Met Ile Arg Ser Arg Asp Tyr Asp Leu Val Ile Ser Asp Met Ser
50 55 60
Met Pro Asp Gly Ser Gly Leu Asp Leu Ile Lys Ile Ile Lys Gln Ser
65 70 75 80
Ser Pro His Thr Pro Val Leu Val Val Thr Ala Tyr Gly Ser Ile Glu
85 90 95
Asn Ala Val Glu Ala Met His Gln Gly Ala Phe Asn Tyr Leu Thr Lys
100 105 110
Pro Phe Ser Ser Glu Ala Leu Phe Ala Phe Ile Ser Lys Ala Glu Glu
115 120 125
Leu Lys Asn Leu Val His Glu Asn Leu Phe Leu His Ser Gln Thr Thr
130 135 140
Pro Asp Ser His Pro Leu Ile Ala Glu Ser Lys Ala Met Lys Asp Leu
145 150 155 160
Leu Ala Ile Ala Lys Lys Ala Ala Ser Ser Ser Ala Asn Ile Phe Ile
165 170 175
His Gly Glu Ser Gly Cys Gly Lys Glu Val Leu Ser Phe Phe Ile His
180 185 190
His Asn Ser Pro Arg Ala Asn His Pro Tyr Ile Lys Val Asn Cys Ala
195 200 205
Ala Ile Pro Glu Thr Leu Leu Glu Ser Glu Leu Phe Gly His Glu Lys
210 215 220
Gly Ala Phe Thr Gly Ala Thr Thr Lys Lys Ala Gly Arg Phe Glu Leu
225 230 235 240
Ala His Lys Gly Thr Leu Leu Leu Asp Glu Ile Thr Glu Val Pro Val
245 250 255
Asn Leu Gln Ala Lys Leu Leu Arg Ala Ile Gln Glu Lys Glu Ile Glu
260 265 270
His Leu Gly Gly Thr Lys Thr Leu Ser Val Asp Val Arg Ile Leu Ala
275 280 285
Thr Ser Asn Arg Lys Leu Lys Glu Ala Ile Asp Asp Lys Ser Phe Arg
290 295 300
Gln Asp Leu Tyr Tyr Arg Leu Asn Val Ile Pro Leu His Leu Pro Pro
305 310 315 320
Leu Arg Asp Arg Gln Asp Asp Ile Leu Pro Leu Ala Asn Tyr Phe Leu
325 330 335
Asn Lys Phe Cys Arg Met Asn Asn Thr Pro Leu Lys Thr Leu Ser Pro
340 345 350
Lys Ala Gln Glu Leu Leu Leu Asn Tyr Pro Trp Pro Gly Asn Ile Arg
355 360 365
Glu Leu Ser Asn Val Leu Glu Arg Val Val Ile Leu Glu Asn Thr Ser
370 375 380
Leu Leu Thr Glu Asp Met Leu Ala Leu Ala
385 390
<210> SEQ ID NO 37
<211> LENGTH: 696
<212> TYPE: DNA
<213> ORGANISM: Chlamydia pneumoniae
<400> SEQUENCE: 37
atgacaaaac atggaaaacg tatacgaggc atcttaaaga actatgattt ctcaaaatca 60
tattctttgc gggaggctat agatatttta aaacaatgtc ctccagtacg cttcgatcaa 120
actgtagatg tatctatcaa gttagggata gatcctaaaa agagcgacca acaaattcgt 180
ggagccgttt ttttacctaa tggtacagga aaaactttaa gaattttggt ttttgcttca 240
gggaacaaag tcaaagaagc tgttgaagcg ggcgcagact ttatgggaag cgacgatctt 300
gttgaaaaaa ttaaatccgg gtggctggaa ttcgatgttg ctgtcgctac cccagatatg 360
atgcgtgaag taggaaaatt aggaaaagtc ttaggaccta gaaatctaat gcctacacct 420
aaaacaggaa cggtaaccac agacgttgct aaagcaatct ccgaattgcg taaaggaaaa 480
attgaattta aagcagaccg cgcaggcgta tgtaatgtag gcgtaggtaa gttgtctttt 540
gaaagcagtc aaatcaaaga aaatattgaa gctctaagtt ctgctttaat taaggccaaa 600
cctcctgcag ctaaaggtca atatttagtc tcattcacta tttcttccac tatggggcct 660
ggtatttcta tagatactag agaattaatg gcatct 696
<210> SEQ ID NO 38
<211> LENGTH: 232
<212> TYPE: PRT
<213> ORGANISM: Chlamydia pneumoniae
<400> SEQUENCE: 38
Met Thr Lys His Gly Lys Arg Ile Arg Gly Ile Leu Lys Asn Tyr Asp
1 5 10 15
Phe Ser Lys Ser Tyr Ser Leu Arg Glu Ala Ile Asp Ile Leu Lys Gln
20 25 30
Cys Pro Pro Val Arg Phe Asp Gln Thr Val Asp Val Ser Ile Lys Leu
35 40 45
Gly Ile Asp Pro Lys Lys Ser Asp Gln Gln Ile Arg Gly Ala Val Phe
50 55 60
Leu Pro Asn Gly Thr Gly Lys Thr Leu Arg Ile Leu Val Phe Ala Ser
65 70 75 80
Gly Asn Lys Val Lys Glu Ala Val Glu Ala Gly Ala Asp Phe Met Gly
85 90 95
Ser Asp Asp Leu Val Glu Lys Ile Lys Ser Gly Trp Leu Glu Phe Asp
100 105 110
Val Ala Val Ala Thr Pro Asp Met Met Arg Glu Val Gly Lys Leu Gly
115 120 125
Lys Val Leu Gly Pro Arg Asn Leu Met Pro Thr Pro Lys Thr Gly Thr
130 135 140
Val Thr Thr Asp Val Ala Lys Ala Ile Ser Glu Leu Arg Lys Gly Lys
145 150 155 160
Ile Glu Phe Lys Ala Asp Arg Ala Gly Val Cys Asn Val Gly Val Gly
165 170 175
Lys Leu Ser Phe Glu Ser Ser Gln Ile Lys Glu Asn Ile Glu Ala Leu
180 185 190
Ser Ser Ala Leu Ile Lys Ala Lys Pro Pro Ala Ala Lys Gly Gln Tyr
195 200 205
Leu Val Ser Phe Thr Ile Ser Ser Thr Met Gly Pro Gly Ile Ser Ile
210 215 220
Asp Thr Arg Glu Leu Met Ala Ser
225 230

We claim:

1. A method of immunizing an animal comprising the step of:
administering a Chlamydia pneumoniae antigen to an animal in an amount effective to induce an immune response against Chlamydia pneumoniae; wherein the Chlamydia pneumoniae antigen comprises the amino acid sequence as set forth as SEQ ID NO:4, SEQ ID NO:6, SEQ ID NO:10, SEQ ID NO:12, SEQ ID NO:16, SEQ ID NO:20 or SEQ ID NO:22.

2. The method of claim 1, wherein the Chlamydia pneumoniae antigen comprises the amino acid sequence as set forth as SEQ ID NO:4.

3. The method of claim 2, wherein the Chlamydia pneumoniae antigen comprises the amino acid sequence as set forth as SEQ ID NO:2.

4. The method of claim 1, wherein the Chlamydia pneumoniae antigen comprises the amino acid sequence as set forth as SEQ ID NO:6.

5. The method of claim 1, wherein the Chlamydia pneumoniae antigen comprises the amino acid sequence as set forth as SEQ ID NO:10.

6. The method of claim 1, wherein the Chlamydia pneumoniae antigen comprises the amino acid sequence as set forth as SEQ ID NO:12.

7. The method of claim 5, wherein the Chlamydia pneumoniae antigen comprises the amino acid sequence as set forth as SEQ ID NO:8.

8. The method of claim 6, wherein the Chlamydia pneumoniae antigen comprises the amino acid sequence as set forth as SEQ ID NO:8.

9. The method of claim 1, wherein the Chlamydia pneumoniae antigen comprises the amino acid sequence as set forth as SEQ ID NO:16.

10. The method of claim 9, wherein the Chlamydia pneumoniae antigen comprises the amino acid sequence as set forth as SEQ ID NO:14.

11. The method of claim 1, wherein the Chlamydia pneumoniae antigen comprises the amino acid sequence as set forth as SEQ ID NO:20.

12. The method of claim 11, wherein the Chlamydia pneumoniae antigen comprises the amino acid sequence as set forth as SEQ ID NO:18.

13. The method of claim 1, wherein the Chlamydia pneumoniae antigen comprises the amino acid sequence as set forth as SEQ ID NO:22.

14. The method of claim 1, wherein the Chlamydia pneumoniae antigen comprises the amino acid sequence as set forth as SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22.

15. The method of claim 1, wherein the method further comprises the step of:
administering a second Chlamydia pneumoniae antigen to an animal in an amount effective to induce an immune response against Chlamydia pneumoniae, wherein the second Chlamydia pneumoniae antigen is different than the first administered Chlamydia pneumoniae antigen and comprises the amino acid sequence as set forth as SEQ ID NO:4, SEQ ID NO:6, SEQ ID NO:10, SEQ ID NO:12, SEQ ID NO:16, SEQ ID NO:20 or SEQ ID NO:22.

16. The method of claim 15, wherein the second Chlamydia pneumoniae antigen comprises the amino acid sequence as set forth as SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22.

17. The method of claim 1 wherein the step of preparing a Chlamydia pneumoniae antigen further comprises preparing the Chlamydia pneumoniae antigen in a pharmaceutically acceptable carrier and wherein the animal is a human.

18. The method of claim 15 wherein the steps of preparing a Chlamydia pneumoniae antigen and preparing a second Chlamydia pneumoniae antigen further comprises preparing the Chlamydia pneumoniae antigen and the second Chlamydia pneumoniae antigen in a pharmaceutically acceptable carrier.

19. The method of claim 15 wherein the step of administering the second Chlamydia pneumoniae antigen comprises administering the second antigen simultaneously with the administration of the first antigen.

20. The method of claim 15 wherein the step of administering the second Chlamydia pneumoniae antigen comprises administering the second antigen subsequent to the administration of the first antigen.

21. The method of claim 15 wherein the step of administering the second Chlamydia pneumoniae antigen comprises administering the second antigen prior to administration of the first antigen.

22. The method of claim 15, wherein the first Chlamydia pneumoniae antigen comprises SEQ ID NO:4 and the second Chlamydia pneumoniae antigen comprises SEQ ID NO:6.

23. A vaccine comprising: a pharmaceutically acceptable carrier, and at least one polynucleotide having a Chlamydia pneumoniae sequence of SEQ ID NO:3, SEQ ID NO:5, SEQ ID NO:9, SEQ ID NO:11, SEQ ID NO:15, SEQ ID NO:19 or SEQ ID NO:21.

24. The vaccine of claim 23 wherein the polynucleotide has a Chlamydia pneumoniae sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or SEQ ID NO:21.

25. The vaccine of claim 23 wherein the polynucleotide has a Chlamydia pneumoniae sequence of SEQ ID NO:3.

26. The vaccine of claim 25 wherein the polynucleotide has a Chlamydia pneumoniae sequence of SEQ ID NO:1.

27. The vaccine of claim 23 wherein the polynucleotide has a Chlamydia pneumoniae sequence of SEQ ID NO:5.

28. The vaccine of claim 23 wherein the polynucleotide has a Chlamydia pneumoniae sequence of SEQ ID NO:9.

29. The vaccine of claim 23 wherein the polynucleotide has a Chlamydia pneumoniae sequence of SEQ ID NO:11.

30. The vaccine of claim 28 wherein the polynucleotide has a Chlamydia pneumoniae sequence of SEQ ID NO:7.

31. The vaccine of claim 29 wherein the polynucleotide has a Chlamydia pneumoniae sequence of SEQ ID NO:7.

32. The vaccine of claim 23 wherein the polynucleotide has a Chlamydia pneumoniae sequence of SEQ ID NO:15.

33. The vaccine of claim 32 wherein the polynucleotide has a Chlamydia pneumoniae sequence of SEQ ID NO:13.

34. The vaccine of claim 23 wherein the polynucleotide has a Chlamydia pneumoniae sequence of SEQ ID NO:19.

35. The vaccine of claim 34 wherein the polynucleotide has a Chlamydia pneumoniae sequence of SEQ ID NO:17.

36. The vaccine of claim 23 wherein the polynucleotide has a Chlamydia pneumoniae sequence of SEQ ID NO:21.

37. The vaccine of claim 23 wherein the polynucleotide is comprised of a genetic immunization vector.

38. The vaccine of claim 23 wherein the polynucleotide is cloned into a viral expression vector.

39. The vaccine of claim 38, wherein the viral expression vector is selected from the group consisting of adenovirus, adeno-associated virus, retrovirus and herpes-simplex virus.

40. The vaccine of claim 23, comprising at least a first polynucleotide having a Chlamydia pneumoniae sequence and second polynucleotide having a Chlamydia pneumoniae sequence, wherein the first polynucleotide and the second polynucleotide have different sequences.

41. A vaccine comprising: a pharmaceutically acceptable carrier, and at least one Chlamydia pneumoniae antigen, at least one Chlamydia pneumoniae antigen comprising the amino acid sequence of SEQ ID NO:4, SEQ ID NO:6, SEQ ID NO:10, SEQ ID NO:12, SEQ ID NO:16, SEQ ID NO:20 or SEQ ID NO:22.

42. The vaccine of claim 41 wherein the at least one Chlamydia pneumoniae antigen comprises the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22.

43. The vaccine of claim 41 wherein the at least one Chlamydia pneumoniae antigen comprises the amino acid sequence of SEQ ID NO:4.

44. The vaccine of claim 43 wherein the at least one Chlamydia pneumoniae antigen comprises the amino acid sequence of SEQ ID NO:2.

45. The vaccine of claim 41 wherein the at least one Chlamydia pneumoniae antigen comprises the amino acid sequence of SEQ ID NO:6.

46. The vaccine of claim 41 wherein the at least one Chlamydia pneumoniae antigen comprises the amino acid sequence of SEQ ID NO:10.

47. The vaccine of claim 41 wherein the at least one Chlamydia pneumoniae antigen comprises the amino acid sequence of SEQ ID NO:12.

48. The vaccine of claim 46 wherein the at least one Chlamydia pneumoniae antigen comprises the amino acid sequence of SEQ ID NO:8.

49. The vaccine of claim 47 wherein the at least one Chlamydia pneumoniae antigen comprises the amino acid sequence of SEQ ID NO:8.

50. The vaccine of claim 41 wherein the at least one Chlamydia pneumoniae antigen comprises the amino acid sequence of SEQ ID NO:16.

51. The vaccine of claim 50 wherein the at least one Chlamydia pneumoniae antigen comprises the amino acid sequence of SEQ ID NO:14.

52. The vaccine of claim 41 wherein the at least one Chlamydia pneumoniae antigen comprises the amino acid sequence of SEQ ID NO:20.

53. The vaccine of claim 52 wherein the at least one Chlamydia pneumoniae antigen comprises the amino acid sequence of SEQ ID NO:18.

54. The vaccine of claim 41 wherein the at least one Chlamydia pneumoniae antigen comprises the amino acid sequence of SEQ ID NO:22.

55. The vaccine of claim 41, comprising at least a first Chlamydia pneumoniae antigen and second Chlamydia pneumoniae antigen, wherein the first polynucleotide and the second polynucleotide have different sequences and comprise SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22.

56. A method of preparing antibodies against a Chlamydia pneumoniae antigen, the method comprising the steps of: (a) selecting a Chlamydia pneumoniae antigen that confers immune resistance against Chlamydia pneumoniae infection when challenged with Chlamydia pneumoniae, the Chlamydia pneumoniae antigen comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38; (b) generating an immune response in a vertebrate animal with the antigen selected in step (a); and (c) obtaining antibodies produced in the animal.

57. A method for assaying for the presence of Chlamydia pneumoniae infection in an animal comprising: (a) obtaining an antibody directed against a Chlamydia pneumoniae antigen; (b) obtaining a sample from the animal; (c) admixing the antibody with the sample; and (d) assaying the sample for antigen-antibody binding, wherein the antigen-antibody binding indicates Chlamydia pneumoniae infection in the animal, and further wherein the Chlamydia pneumoniae antigen has a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38.

58. The method of claim 57, wherein the antibody is a monoclonal antibody and the animal is a human.

59. The method of claim 57, wherein the step of assaying the sample for antigen-antibody binding is accomplished by precipitin reaction, radioimmunoassay, ELISA, Western Blot or immunofluorescence.

60. The method of claim 57, wherein the step of obtaining an antibody comprises the steps of: (a) selecting a Chlamydia pneumoniae antigen that confers immune resistance against Chlamydia pneumoniae infection when challenged with Chlamydia pneumoniae, the Chlamydia pneumoniae antigen comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38; (b) generating an immune response in a vertebrate animal with the antigen selected in step (a); and (c) obtaining antibodies produced in the animal.

61. A kit for assaying for a Chlamydia pneumoniae infection, the kit contained in a suitable container, and comprising an antibody directed against Chlamydia pneumoniae, wherein the antibody binds to a Chlamydia pneumoniae antigen comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38.

62. A method of immunizing an animal comprising the step of:
administering at least three Chlamydia pneumoniae antigens to a human in an amount effective to induce an immune response against Chlamydia pneumoniae; wherein the at least three Chlamydia pneumoniae antigens are distinct from one another and each comprises an amino acid sequence selected from SEQ ID NO:4, SEQ ID NO:6, SEQ ID NO:10, SEQ ID NO:12, SEQ ID NO:16, SEQ ID NO:20 or SEQ ID NO:22.

63. The method of claim 62, wherein the at least three Chlamydia pneumoniae antigens comprise the amino acid sequences as set forth as SEQ ID NO:4, SEQ ID NO:6 and SEQ ID NO:10.

64. The method of claim 62, wherein the at least three Chlamydia pneumoniae antigens comprise the amino acid sequences as set forth as SEQ ID NO:4, SEQ ID NO:6 and SEQ ID NO:12.

65. The method of claim 62, wherein the at least three Chlamydia pneumoniae antigens comprise the amino acid sequences as set forth as SEQ ID NO:4, SEQ ID NO:6 and SEQ ID NO:16.

66. The method of claim 62, wherein the at least three Chlamydia pneumoniae antigens comprise the amino acid sequences as set forth as SEQ ID NO:4, SEQ ID NO:6 and SEQ ID NO:20.

67. The method of claim 62, wherein the at least three Chlamydia pneumoniae antigens comprise the amino acid sequences as set forth as SEQ ID NO:4, SEQ ID NO:6 and SEQ ID NO:22.

68. The method of claim 62, wherein two of the at least three Chlamydia pneumoniae antigens comprise the amino acid sequences as set forth as SEQ ID NO:4 and SEQ ID NO: 6 and the at least one additional antigen is selected from the group consisting of: SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22.

69. The method of claim 62, wherein two of the at least three Chlamydia pneumoniae antigens comprise the amino acid sequences as set forth as SEQ ID NO: 4 and SEQ ID NO: 6 and the at least one additional antigen is selected from the group consisting of: SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38.

70. The method of claim 62, wherein two of the at least three Chlamydia pneumoniae antigens comprise an amino acid sequence selected from the group consisting of: SEQ ID NO:4, SEQ ID NO:6, SEQ ID NO:10, SEQ ID NO:12, SEQ ID NO:16, SEQ ID NO:20 or SEQ ID NO:22, and the at least one additional antigen is selected from the group consisting of: SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38.

71. The method of claim 62 wherein the step of preparing the at least three Chlamydia pneumoniae antigens further comprises preparing the at least three Chlamydia pneumoniae antigens in a pharmaceutically acceptable carrier.
